(19) United States
(12) Patent Application Publication
(10) Pub. No.: US 2026/0117180 A1
(43) Pub. Date: Apr. 30, 2026

(54) UT2 GENE KNOCKOUT ERYTHROID PROGENITOR CELLS AND METHODS OF ERYTHROID DIFFERENTIATION THEREOF

(71) Applicant: PUSAN NATIONAL UNIVERSITY INDUSTRY-UNIVERSITY COOPERATION FOUNDATION, Busan (KR)

(72) Inventors: Dongjun Lee, Yangsan-si (KR); Jiwon KIM, Yangsan-si (KR); Hee Eun BAE, Busan (KR); Hee Seon CHOI, Yangsan-si (KR)

(73) Assignee: PUSAN NATIONAL UNIVERSITY INDUSTRY-UNIVERSITY COOPERATION FOUNDATION, Busan (KR)

(21) Appl. No.: 19/303,358

(22) Filed: Aug. 19, 2025

Related U.S. Application Data
(63) Continuation of application No. PCT/KR2025/008443, filed on Jun. 18, 2025.

(30) Foreign Application Priority Data
Oct. 24, 2024 (KR) 10-2024-0146580

(51) Int. Cl. C12N 5/078; C12N 9/22; C12N 15/11; C12N 15/86; C12N 15/90

(52) U.S. Cl. CPC C12N 5/0641; C12N 9/226; C12N 15/11; C12N 15/86; C12N 15/907; C12N 2310/20; C12N 2500/24; C12N 2500/40; C12N 2501/125; C12N 2501/14; C12N 2501/33; C12N 2501/39; C12N 2506/11; C12N 2510/00; C12N 2740/15043

(57) ABSTRACT

The present disclosure relates to a UT2 gene-knockout erythroid progenitor cell and a method of differentiating the same into an erythrocyte, wherein it has been determined that UT2 gene-knockout cells, in which a UT2 gene is knocked out in erythroid progenitor cells, express erythroid differentiation markers at a higher rate and undergo erythroid differentiation more rapidly under erythroid differentiation conditions according to the present disclosure, such that the present disclosure provides a method that enables rapid differentiation of erythrocytes using UT2 gene-knockout erythroid progenitor cells.

### CROSS-REFERENCE TO RELATED APPLICATION

This application is a Continuation of PCT International Patent Application No. PCT/KR2025/008443 filed on Jun. 18, 2025, which claims priority to Korean Patent Application No. 10-2024-0146580 filed on Oct. 24, 2024 which are all hereby incorporated by reference in their entirety.

### SEQUENCE LISTING

This application contains a Sequence Listing submitted via USPTO Patent Center and hereby incorporated by reference in its entirety. The Sequence Listing is named 2280-600.xml, created on Aug. 16, 2025, and 154,923 bytes in size.

### BACKGROUND OF THE INVENTION

The present application is supported by the National Research Foundation of Korea under Ministry of Science and ICT, Ministry of Trade, Industry and Energy, Ministry of Health & Welfare, Ministry of Food and Drug Safety for the following research projects:
1) A study on development of new concept erythrocyte differentiation and proliferation restoration technology through identification of differentiation dynamics of highly efficient mobilized hematopoietic stem progenitor cells based on niche enhancing factors (RS-2023-KH140743).
2) A study on research center for molecular control of cancer cell diversity (2022R1A5A2027161).
3) A study on mechanical study on the differentiation control of immature leukemia/cancer stem cells based on key factors and treatment of blood cancer in the bone marrow microenvironment (RS-2024-00333287).

### 1. FIELD OF THE INVENTION

The present disclosure relates to a UT2 gene-knockout erythroid progenitor cell and a method for differentiating the same into an erythrocyte.

### 2. DESCRIPTION OF THE RELATED ART

Erythrocytes are the most abundant cellular component of blood, constituting approximately 44% of total blood cells, and primarily function to transport oxygen. Erythrocytes are produced from hematopoietic stem cells through several differentiation stages, where the nucleus is gradually condensed and the cell size decreases after going through burst forming unit erythroid (BFU-E), colony forming unit erythroid (CFU-E), proerythroblast, basophilic erythroblast, polychromatic erythroblast, and orthochromatic erythroblast. After the condensed nucleus is enucleated, it becomes a reticulocyte, and as the remaining RNA and microorganelles are removed and it takes on a biconcave shape, it becomes a mature erythrocyte (RBC). This differentiation process typically takes place over three weeks in vitro, and cell proliferation is limited after the polychromatic erythroblastic stage where mature erythroblasts are predominant.

Erythrocytes are essential in emergency situations such as bleeding and in pathological, medical conditions such as cancer, blood, and surgery. Though erythrocytes are collected through blood transfusions worldwide, there are limits to the transfusion of erythrocytes with suitable conditions, and problems such as contamination with pathogens and viruses occur during the process of collecting and transporting blood, such that various attempts are being developed to provide safe blood. One of the attempts is the creation of artificial blood, the basis of which is the production of erythrocytes in a test tube. A known method for producing erythrocytes in vitro is to use a differentiation medium from regulatory stem cells. However, the current method has a critical shortcoming in terms of large-scale production, with an issue that proper maturation of erythrocytes does not take place.

### SUMMARY OF THE INVENTION

### Problem to be Solved by the Invention

An object of the present disclosure is to provide a method of preparing a UT2 gene-knockout erythroid progenitor cell and differentiating the same into an erythrocyte.

### Means for Solving the Problem

The present disclosure provides a method for erythroid differentiation comprising: knocking out a urea transporter 2 (UT2) gene in erythroid progenitor cells in vitro; and culturing the UT2 gene-knockout erythroid progenitor cells in an erythroid differentiation medium to differentiate the erythroid progenitor cells into erythrocytes.

In addition, the present disclosure provides an erythrocyte differentiated according to the method.

### Effects of the Invention

According to the present disclosure, it has been determined that UT2 gene-knockout cells, in which a UT2 gene is knocked out in erythroid progenitor cells, express erythroid differentiation markers at a higher rate and undergo erythroid differentiation more rapidly under erythroid differentiation conditions according to the present disclosure, such that it is possible to provide a method that enables rapid differentiation into erythrocytes using UT2 gene-knockout erythroid progenitor cells.

### BRIEF DESCRIPTION OF THE DRAWINGS

FIG. 1 shows an experimental schematic for establishing a UT2 gene-knockout cell line from erythroid progenitor cells using the CRISPR-Cas9 system.

FIG. 2 shows a result of analyzing whether a UT2 gene is knocked out in UT2 gene-knockout cells established according to the present disclosure via a T7 Endonuclease 1 (T7E1) detection method.

FIG. 3 shows an experimental schematic for differentiating UT2 gene-knockout erythroid progenitor cells established according to the present disclosure into erythrocytes.

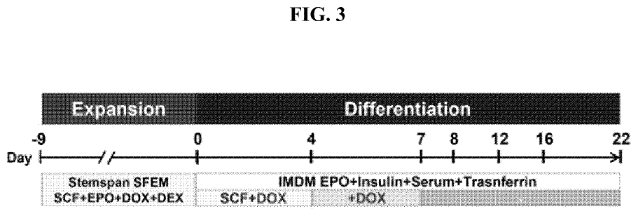

FIG. 4 shows results of analyzing changes in an expression level of erythroid differentiation markers after differentiating UT2 gene-knockout erythroid progenitor cells established according to the present disclosure into erythrocytes.

### DETAILED DESCRIPTION OF THE INVENTION

The terms used in this specification are selected from general terms that are most widely used currently as much as possible while taking into account the functions of the present disclosure, but these may vary depending on the intention of those skilled in the art, precedents, or the emergence of new technologies. Additionally, in certain cases, there are terms arbitrarily selected by the applicant, and in such cases, meanings thereof will be described in detail in the description of the relevant disclosure. Therefore, the terms used herein should not be simply defined as names of terms, but based on the meaning of the terms and the overall content of the present disclosure.

Unless otherwise defined, all terms used herein, including technical or scientific terms, have the same meaning as commonly understood by those of ordinary skill in the art to which the present disclosure pertains. Terms defined in commonly used dictionaries should be interpreted to have a meaning consistent with those in the context of the relevant art and not be interpreted in an idealized or overly formal sense unless expressly defined in this application.

Hereinafter, the present disclosure will be described in more detail.

The present disclosure provides a method for erythroid differentiation, comprising: knocking out a urea transporter 2 (UT2) gene in erythroid progenitor cells in vitro; and culturing the UT2 gene-knockout erythroid progenitor cells in an erythroid differentiation medium to differentiate the erythroid progenitor cells into erythrocytes.

The progenitor cells are undifferentiated cells with a self-replicability and differentiation capacity, but are ultimately differentiated with a cell type to be differentiated predetermined. As used herein, the term “erythroid progenitor cell,” unless otherwise specified, is intended to include erythroid precursor cells. The erythroid precursor cells refer to all cells involved in the process of erythrocyte formation except mature erythrocytes. Specifically, the erythroid progenitor cell may refer to an erythroid cell before enucleation and may be an immature erythroid progenitor cell or a glycophorin A-positive cell prior to enucleation, where the glycophorin A is a molecule specific to the erythroid lineage. For example, the erythroid progenitor cells may be selected from the group consisting of proerythroblasts, basophilic erythroblasts, polychromatic erythroblasts, and orthochromatic erythroblasts.

The erythroid progenitor cell is cultured in a medium including a stem cell factor (SCF), erythropoietin (EPO), doxycycline (DOX), and dexamethasone (DEX).

The knocking out of the UT2 gene is to knock out the UT2 gene (Genebank ID: NC_000014.9) represented by SEQ ID NO: 1 via the CRISPR (Clustered regularly interspaced short palindromic repeats)/Cas9 system using sgRNA, and the UT2 sgRNA is introduced into erythroid progenitor cells by lentivirus.

The UT2 gene-knockout erythroid progenitor cells are selected from a medium containing puromycin.

In the knocking out of the UT2 gene, the lentivirus containing an sgRNA that complementarily binds to the UT2 gene represented by SEQ ID NO: 1 is added to the erythroid progenitor cells, followed by culture for 24 to 48 hours.

The differentiating into erythrocytes consists of culturing the UT2 gene-knockout erythroid progenitor cells in a primary differentiation medium containing erythropoietin (EPO), insulin, transferrin, doxycycline (DOX), and a stem cell factor (SCF) for 4 days; culturing the UT2 gene-knockout erythroid progenitor cells for 3 days in a secondary differentiation medium containing erythropoietin (EPO), insulin, transferrin, and doxycycline (DOX); and culturing the UT2 gene-knockout erythroid progenitor cells in a third differentiation medium containing erythropoietin (EPO), insulin, and transferrin.

The differentiating of the erythroid progenitor cells into erythrocytes is completed within 7 to 22 days.

In addition, the present disclosure provides an erythrocyte differentiated according to the method.

Hereinafter, in order to help understand the present disclosure, experimental examples and examples will be described in detail. However, the following experimental examples and examples are only illustrative of the contents of the present disclosure, and the scope of the present disclosure is not limited to the following experimental examples and examples. The experimental examples and examples of the present disclosure are provided to more completely explain the present disclosure to those skilled in the art.

### [Example 1] Establishment of UT2 Gene-Knockout Cells

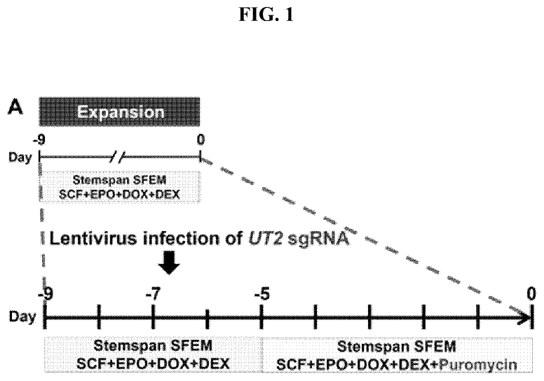

To identify the role of the urea transporter 2 (UT2) gene in erythroid differentiation, a UT2 gene-knockout cell line was established in HUDEP-2 cells, a human erythroid progenitor cell line, using the CRISPR-Cas9 system in the same manner as in FIG. 1. HUDEP-2 cells were cultured in Stemspan SFEM (Stemspan™ Serum-Free Expansion Medium, STEM CELL technologies) medium containing 0.4 μg/ml dexamethasone (DEX, Sigma), 1 μg/ml doxycycline (DOX, Clontech), 50 ng/ml stem cell factor (SCF, R&D systems), and 5 ng/ml erythropoietin (EPO, Peprotech). To knock out the UT2 gene in HUDEP-2 cells grown in a medium containing cytokines, HUDEP-2 cells were transformed via the spin infection method using UT2 sgRNA lentivirus. After adding 0.8 μg/ml polybrene (Santacruz), a chemical that helps viral infection, to the Stemspan SFEM medium containing cytokines, 1×10⁶ HUDEP-2 cells were suspended in 2 mL of the medium. After dispensing into a 6-well plate, 1 mL of 2×10⁶ TU/ml lentivirus containing UT2 sgRNA (sc-414550-ACT, Santa Cruz) was added. Centrifugation was performed for 90 minutes at 20° C. and 2,000 rpm. After 24 hours, the cells were resuspended in 2 mL of fresh cell culture medium containing polybrene, and 1 mL of UT2 sgRNA lentivirus was added, followed by centrifugation once more. After 24 hours, the medium was replaced with Stemspan SFEM medium containing cytokines. After culturing for 24 hours, the medium was replaced with those containing 0.5 μg/ml puromycin and cultured for 5 days to screen only virus-infected cells.

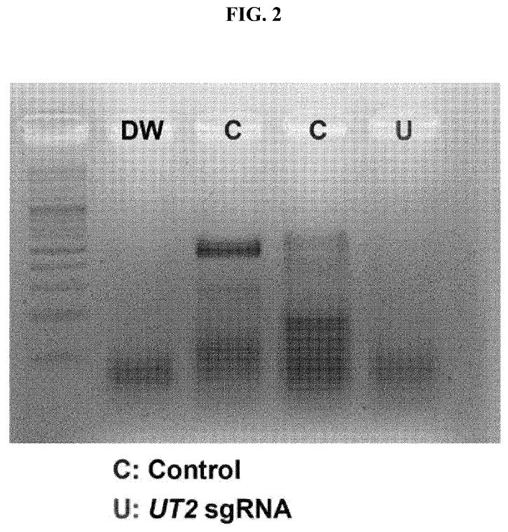

To determine whether the screened HUDEP-2 cells had undergone UT2 gene manipulation compared to the control group in which no viral transformation took place, the T7 Endonuclease 1 (T7E1) detection method was used to evaluate whether the UT2 gene was knocked out. DNA was extracted from cells using the Geneall DNA extraction kit. The extracted DNA was used to identify the presence of insertion and deletion (Indel) of the UT2-knockout gene in HUDEP-2 cells via the T7E1 detection assay. Indel primers used were the sgUT2 indel forward primer of SEQ ID NO: 2 (AATTCAAGAAGATGGGAGCCTCTG) and the sgUT2 indel reverse primer of SEQ ID NO: 3 (AATTCTCATCCAGCGAGTCTGCAT). As shown in FIG. 2, reduction in the expression of UT2 gene was detected in HUDEP-2 cells applied with the CRISPR-Cas9 system compared to control cells with no viral transduction via the T7E1 assay.

### [Example 2] Differentiation of UT2 Gene-Knockout Cells into Erythrocytes

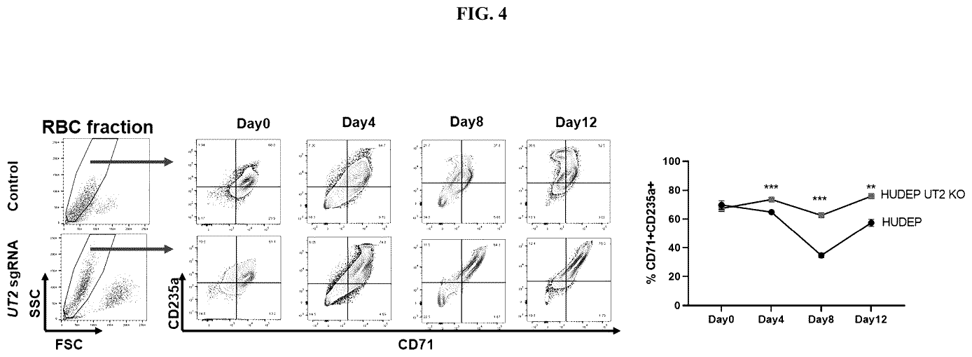

An evaluation was conducted on whether the erythroid differentiation potential of the UT2 gene-knockout erythroid progenitor cells differs. To differentiate erythroid progenitor cells into erythrocytes, the erythroid progenitor cells were cultured for 4 days in Iscove's Modified Dulbecco's Medium (IMDM) containing 5% human AB serum (Sigma), 400 μg/mL holo-transferrin (Sigma), 10 μg/mL human insulin (Sigma), 5 ng/mL erythropoietin (EPO, Peprotech), 1 μg/mL doxycycline (DOX, Clontech), 41.25 μg/mL heparin (Sigma), and 50 ng/mL stem cell factor (SCF, R&D systems). Afterwards, the cells were cultured for 3 days after removing SCF from the IMDM medium, and then cultured in a medium with DOX removed. Erythroid differentiation potential was monitored at Day 4, Day 8, and Day 12 from the start of differentiation (Day 0). After treatment with fluorescence-labeled antibodies, differences in the expression of erythroid differentiation markers CD235a and CD71 were analyzed via flow cytometry. As shown in FIG. 4, compared to the control group, the expression of erythroid differentiation markers CD235a⁺ and CD71⁺ was found to be at a higher rate, and the erythroid differentiation was carried out more rapidly in UT2 gene-knockout cells (UT2 sgRNA).

While a specific part of the present disclosure has been described in detail above, it is clear for those skilled in the art that this specific description is merely preferred example embodiments, and the scope of the present disclosure is not limited thereby. In other words, the substantial scope of the present disclosure is defined by the attached claims and their equivalents.

The numerical range includes the numerical values defined in the above range. Any maximum numerical limit given throughout this specification includes any lower numerical limit as if the lower numerical limit were explicitly written out. Any minimum numerical limit given throughout this specification includes any higher numerical limit as if that higher numerical limit were explicitly written out. Any numerical limit given throughout this specification will include any better numerical range within the broader numerical range, as if the narrower numerical limit were explicitly written.

What is claimed is:

1. A method for erythroid differentiation, the method comprising:
knocking out a urea transporter 2 (UT2) gene in erythroid progenitor cells in vitro; and
culturing the UT2 gene-knockout erythroid progenitor cells in an erythroid differentiation medium to differentiate the erythroid progenitor cells into erythrocytes.

2. The method of claim 1, wherein the erythroid progenitor cell is cultured in a medium comprising a stem cell factor (SCF), erythropoietin (EPO), doxycycline (DOX), and dexamethasone (DEX).

3. The method of claim 1, wherein the knocking out of the UT2 gene comprises using the Clustered regularly interspaced short palindromic repeats (CRISPR)/Cas9 system with sgRNA to knock out the UT2 gene (GenBank ID: NC_000014.9) represented by SEQ ID NO: 1.

4. The method of claim 3, wherein the UT2 sgRNA is introduced into the erythroid progenitor cells by lentivirus.

5. The method of claim 1, wherein the UT2 gene-knockout erythroid progenitor cells are selected from a medium comprising puromycin.

6. The method of claim 1, wherein the knocking out of the UT2 gene comprises adding a lentivirus, which comprises an sgRNA that complementarily binds to the UT2 gene represented by SEQ ID NO: 1, to the erythroid progenitor cells, followed by culture for 24 to 48 hours.

7. The method of claim 1, wherein the differentiating into erythrocytes comprises:
(a) culturing the UT2 gene-knockout erythroid progenitor cells in a primary differentiation medium comprising erythropoietin (EPO), insulin, transferrin, doxycycline (DOX), and a stem cell factor (SCF) for 4 days;
(b) culturing the UT2 gene-knockout erythroid progenitor cells for 3 days in a secondary differentiation medium comprising erythropoietin (EPO), insulin, transferrin, and doxycycline (DOX); and
(c) culturing the UT2 gene-knockout erythroid progenitor cells in a third differentiation medium comprising erythropoietin (EPO), insulin, and transferrin.

8. The method of claim 1, wherein the differentiating of the erythroid progenitor cells into erythrocytes is completed within 7 to 22 days.

9. An erythrocyte differentiated by the method according to claim 1.


SEQUENCE LISTING (3 sequences)

SEQ ID NO: 1 - DNA, 151453 bp, Homo sapiens
  source 1..151453: mol_type = genomic DNA
ctccccctagcgtcccagacagcggagggagtcgccgccgctaccgccgccgccgccgcagggcccgccgctgggatgccgagcgcccgcgccgccgctgcctctgtcctccgcgcgctgctcagctgaaggcgcacaggtgaggacgggcccggctccggccacttgggcggccgcggctgcagacgtggcgccggggcagggacggaaccggagcctcagtggcgtggggtcgctccgcgagcgtaacagtcccgggagcgttgcagccctgggtgcgccaggcgtgcgccccgatcctcctccggagcgcggtgcccaggtgtggggtgcgcgggggcagctctgggcgcgctgggcgcggcgcgcaggcctcggaggactcaccgcactcctagctccagcccagcctcccagctccgcaccagcgagccggcctttgtgctggcggagcttgcagggctggctgcttttgtgtgggcttttctttctctttctccctccgctcgcaatcccacagcagtgagagagaagttcccgaaaacaacggtttactcctttcaaaccgtcgatcgcagtgtccccaatgccactcagagcctaacaatcgaggagcgaatcgttgctgcggtcaccaccgaaatatgtcctgccactttgcagaaaacaaacagttgagtgtttagcgcagagatgtcgagcctccttgctcagcccctgactcgtcccccaggacgcggctggaggacgctccttggaaaggtcacttaattagaaagagggcctgcgggttagacctgggggaggcgaggctctgcttgagactccggaatagttgggaggtgctgccggaggtgcccaaatgtctcatgtgcctcgtggtcactgacagaatctgacttgtgtagattccaaacatgtagaaccttggctggcgaggaggcaagcaggagtgaacaaaacaggacccttaagagtttgggttctcaaagatgagatgatcccgaggatccgcctagcacttggcatccaatttcagtgcagaactgatgtggaagagaggagagatggctgctttaaagtgtctgccttctttctttggaagttttagtttgaaacttgagggggagtggggagtggttcacaaaaccatccctagggttctgaaggatctcttctccaaagggcaaaaattcttgagtggtcagaattcacatgccttctctttattttggtgtatttccttgatcacaagtgatgctgttgggctccagggagtctccttttagccaagcatctacagtcattgactgacttcggcacgtatgccagtagaaccccacagtagcatctattgaaacacacaaccggctaaggtttcacagtatcagaacattgcttcttttacacacagaggtttaaaggtactctgacttgctcttatgaataaagtggttttttttttgtatttgtcacaataatgaaccgagcacttgtgcagcagacagatttgatattagttgagtttattcatagaaactcccaaggctcaccaagtctaaggtagctacagcaacggagccaattagagagatgtgaatttgtctttgttttcatgggaggcttcttactacatattccggagtttcctgccagacctttacaatattataatttggtattacggaaatggttcgatgctaaatataagtgtagaatcaaaatgaggaaaacgggtttatctgctgtggaaaatgtccactccccacctaaaggtgtcctttgccctttattctgtagatgtttgtctgaaaagtggtgtatggaattgtatttttgttaattgaacttaaaaaacttctcacatagggaagctgtctatgcagagggaaactgcttttaatttttgtaaaatgaaaactcattccctaagcaagatgtgaggtcaatcttatttttccatattgtgtttacttggtgattataagagaagtatctgaagctagtttcagcttccctttaaatgtcagctacttagagatctagccaagcaaatggctacatttgtgtaccacaaaaaccagaaatgatatgtccaattgggtcactgaaataaactgtctgatggatggagaagagtttgttgatcaagattcatcaggataggattctgcagacagcagaaccctctcaggtcacattgtctacatgataaagctgaaaggagaagctccatgaaagtaccttggggaaaaaaatgctactccatttgtggctgatgttacagatgctccaaattgctcacatttctgtatttgacaggaatgcatgtcagtagcatgctagtatgtcttctattgcatgataatttacttttgtttcaaactgcttttatgtctattatcacttttgtgtgtgttctccaccctttgaaaaaatataaccagcattattatttctactttgcaacaagaaaacttgagtttcagtgatttgcctaaggtagaactggaactcgaagaagcctgtttttaaatcttcaagtccatctccttttaccagggatgagatgatatgctaggggtgataagcacggatatcttcatttgtctacatcactaatttgtagagactgaagctcctgggctcagtatgtagaacttaggtgtggtcttcagggatggtaaagtactttttactggtatttagtttccagtaatttagctattttgaccccagcctacttaccctcttccctgccagctctggggctgctttcctcctgggggaccatgcttttctttgtggttacttactccttctccctccttccattgtgatggaacatgctgctgaaaggattgtttgtcattcagctatttaatttatggtgaagtatagctgcagcctagatcctaaagccaatataacagaccagtgatgattgtcagtagcacagtgaatcagaaacgtggtaccttaatgccatttgccacagctgggttaatagagtgagagtctttataagctcagctttaagtaaatacaatagtattggcaataaaatagtgttggcagtaacagccttaaggatttaaaaaaagaaaaagtaagacaatgataaattaatcattgaggaatggtcatgaaaaggacaaagtggaaagattgttaagacacaaaatggaaagattattaagacacaaagattattaagacactatccccctcatcagagaatcactaattgtatttcaaaaggtgagatggtatttggcaagcatgttgacttgacgctgtgagacttgttacctgtaactgttggtctgttgcgtgtgtgtgtgtgtgtgtgtgtgtgtgtgtgtgtaataaactctgaaaccttaaaatatagcagaaatgagaacacataagttattgttattgttagagtgacagttaaataagtagccaaaattggctaggtattgaataaaacatacaagtagacatggttagaccatgaaatgtcattaaaaaaatcttacagtacaaaataaggacttgaaacgttctttttcatattcttaaatggttatttatattcacatcatgccaacaaatatttatggaatctctcctctttgtgcctacccttattactaattatgtggcaaacaaaggaatggtaaagactagtgtttgtatgcaaagaacataatctcatttgaggtgctgagacttaaatgacaattcatgctacaggagctaggagaagggtggggaaagaatgggccagaatgggatgtgacctgggaggccagggtttggatacctgggtagggaaccatgggtcgtacagtgtcattctcaaaacttgggagcctgagagctgagcacggtggctcacgcctgtaatcccaacactttgggaggccgaggtgggtggatcacgaagccaagagttcgagaccagcctggccaacatggtgaaaccccatttctactaagaatacaaaaattagccgggtgtggtggcacatgcctgttatcctagctactcgggaggctgaggcaggagaattgcttgaacctgggaggtggaggttgtgagccgagatcatgccactgcactgtagcctgggcgacagagcgagactccatctcgaaaataaaacaaacaaaaaaccaacttaggagcctgagatgagtatggcacataaaagtgctatgtatgtgactgatgatggtgtttatactgggcaccagtggtatggggttgccttattagagatggattttggcaattgcatatttggggacattatatgctgtgttttgggaaagcagagagttgtctggcaaaggtagggacgttgaattctgacagaatccaaaagatcaaaaacagacactatcccttaggcaagttacttatcctctaccagcctgttgttttcctcatttaaaaagtggaataacgacagtatacctcattaggttgttgtaaggattaaataagataatttccactgggcacttagaacgcttggcacatagcagctcctaagtggtagctgtctttgattgattattgcaatttttattcttaagaataaaccagctgggtgtggtggctcacacctataatccaagcaatttgggaggctgaggtgggcggatcatgaggtcaagagattgagaccatcctggccaacatggtgaaatgccgtctctactaaaaacacacaaactagctgggcgtggtggcgtgcgcctgtggtcccagctactcaggaggctgaggcaggataatcgcttgaacctgggaggaggaggttgctgtgagctgagatctcgccactgcactccagcatggagacagagtgagacttcctatcaaaaaaaaaaaaaaaaaccatctggattgtataaaatagattggagaggagagagcctagacaaggttatcaatgaagaggcatttaaaataactatccaatgaggtgatcagaagttacaattccaaaagagattatgagaggaggatccacagcggttgtgaatctagaatgtaaaggagagaagagttcaaggtggccaccaaagttcagatgaagatggttgtgtttgaagtgtcatcaaagtaacttccagttgacaatgaaaagccaagatgaggtagctttcttcacctaccccatagcagctctctgtagtttttgtgtatatcataagaaaaggaaaatataaagcgaactgtcaccagtttcttggcacactctacagccctcctgtgtcttgagcactcttggggcgatgctcattgtgccttaatattatatatgaaactcagttgacactggaagtagtagcttcatcactggtgagaatgatttaacatattggtttcccttttgtgtcttcattttggtaacatactgaaatagcagtattcattttagttggaaaaaatattgaccaagtgcttacaaaatctgattaagtctaaccagatggccaaggctggtattttttatgttgcctttcgtttgtctttgaaacttgaatatgcctagacttgacaaagaaacagattctggactgtagctatgtaagattgtcaaagataatgcctgacagaacagcttatcttgctcactggtggcagaaatgcagtaaattgtttagaagactgccaagctattaggatagatagcttgatagcaaatgatagacaaagtgctactgacctgctctgaaggagatgctttttaagctattcaaagctgatatccaacatcagttggtaatttggttagagcttatttatctgaattaatttgcatggagagatacagtgaaaagaaagcagcatccaaatacgcagtgggagggaaaaggaagaggaatgttctttttgtttgtttgttaaacattcttatgattgagttaattgaagataattaagacacagaattctaatatgcaatggaatctctcctccttccacttctcccagcaaagtgggctatagacattcatctgtttgttcagccaatatttactgagcatttcttctgtgccaggcacattgccaggaaggggatacagtgttgaggaaaaaggaaaggctcatagccttcaaaatatggttgcagatggttatttaggtttaggaatattgttgaactgacaacagtcttctcgtttattcttgagcatcatgtaactcccattgtgggacattagaaaaatgggagtgactacatgaacctatactgggctttgaaagatggagaaaatcctggaatttgaaggtcatctatcagtttatctgagttccagacgtgttttctgttattctttttacaggctgtcttttttccttttaacgtatagttgttgctttcgatagtggggagagggaacatatggcctcaatacacagacatagaaaaagctttcatcaatgcctttgaaaaggggggagaaaagcaaaggcttgcggttcacccccactctttctgttgtatttgaatttagaaagcaggtggctgacaatcacagaaggcaagtcttttggagcaatagcagaattcccttcacgcaaaaattctaaaacccaagatacagatgaaaataaacttggaaggaaaattatgttttggaaaggcagtggggctgtattaagtctttattatttatcccttcttcccttgatctgatttgtttgtaaaatttttctctcagtccacatgactccagaacatacacatagcacataaagcatctgctccaccatttgagtgcctttcctagttgatggcaacattataggaatatagaaatatttgtatttttatttcatgtcatataggtgaggtatgagaataaaatcatggatgaagaagggatttgaatgattattttaattcctcattttgtcaaaggagaaactgaggccacttgctcagtatcatatagcaagtttcaatgtcaataaagctaagattcaggaatcatttttctgatgccagcccaagtccctctaaactctattaccattcatgatggcctggggctttcagatttttattcagaagtatgggatcatgtgggccaaggttttaggccacacgtttagtaagatttcactgaatagaaaaagtatttgtttaggaaggggcagctatctgaacagatcagaaatgaaccacagagttctatagcttttatgactgttcgactatatagatagaatttagtaggtcagtttgtctgaacagaaatccgaaggtgtaatttcattgactgtcaattctcttgtttcacagctatcatattttttttgtttgtttttttttgagatggagtcttgttctgccacccaggctggagtgcagtggtgcgatcttggctcacggcaagctcggcctcctgggttcacgccattctcctgcctcagcctcccgagtagctgggactacaggcgcccgccaccatgcccggctaatttttttgtatttttagtggagacggggtttcaccgtgttagccaggatggtctcaatctcctggcctcgtgatccacccgccttggcctcccaaagtgctgggatttcaggcgtgagccactgcgccaggcctcatatgttttgttgttgttgttgttgtctgtttgtttttgagacagggtcttttctctgtcgcccagaatggagtgtagtggtatgatcacggctcactgcagccttgactttctgggctcaagtgattctcccacctcagcctcctgagtaactaggactactggtgtgtgcctggctcattttgtagagatgagatttgccatgttgctcaggctggtctcaaactcctgggctcagatgatcctcctgccttggcctctcaaagtattgggactacaggtatgagccactgcacctggccccttcattacgtttttgttttaaggaaggcaaatagaggcatcaagccaaattcagtgatctgttacctggctatgaactagagtgcatcctggctcatctaaagtacagtgataactaatgtccagaatgttgacatgctgtggcacatgctatttcctctttatcaaatatctttgccattttctcttttttaaaaagaacagtgtggccaggtgtggtggctcacacctgtaatcccagcactttgggaagctgaggtgggtggatcgcttgaggtcaggagttctagatcagcctggccaacatggttaaaccctgtccatactaaaaatacaaaaattagctgggtgtgatggtgggcacctgtaatcccagctacttaggaaattgaggcaggagaatcacttcaacctggaaggtggagggagcagtgagctgagatcacaccatcaaactccgacctgggtgacagagcaagactctgtctcagaaaaaaaaaaaaaaaaaaaaaaaagaggatgggcacggtggctcatgcctgtaatcctagcactctgggaggttgaggtgggtggatcacttggggtcagaagttcgagaccaacctggccagcatggtgaaaccctgtctctattaaaaatacaaaaactagccaggcatggtagtgagcacctgtaatcccacctatttgggaggctgaggcaggagaatcacttgaacctgggtgacagaggttgcagcgagccgagatggcatcagtgcactccagcctgggcaacagagtgagactccatctcaaaaataaacatacaaataaacaaaaacaatgctagctaccagcatgtaatttaatattcagatatgtttgtttcaaacccccatcagcatatggggaaacttagcacccgtctcattatccttgcagattgtttggggacttatgtttcttctagtcatctctggcttcagttttcttcagctaaatttgtaatccctgataggctgtttcttttctgcacatgacactattgtactcgtgctgtcattccattcctgtatttccctgcagcattgtggactcaagatttctgtgtaacaaattaccccgtagcttagtggcttgaaataacaataaatatttgttattttataccagttctatgcatcatgaatttgggagtaacttagctgtgtggttatggcacaaagtctttcaagaaggtgtagtcaagatgtcagccaggggtgcagtcatctgaagggttgactggggctgctggacccatttccaagatggtacactcatgtggtggtggtagttggcaggaagccttagctctaaatccatggattccttttttctcacgtagaaaaagtcagtcttcttgtgctgtgtggtggtatgtgcctgtagtcctacctacttaggagcttgatgcaggaggattgcttgagcccaggagttcaagtccagcctggggaaagaaaaaaagagaagaaagggaaaggaaggggaggggagctcacgcttgtaatttcagcactttgggaggctgaggcaggtggatcacttgaagtcaggagttcaagaccagtctggccaacatggtgaaaccccgtctctaataaaaatacaaaaattagccaggcatggtggtgcacgcctgtagtcccagctactcgggaggctgaggcacaagaactcaagaggcggaggttgcagtgagtgactcgcactgcattccagcctgggtgacagagtgaaactccatctcaaaaagaaaagaaaaaagaaaagaaaagagaagagaaaagaaaagaaaagaaacaaaaaaaggaggaaggaaggaaaattccggcttctttttgtcttcagccatgtagatcatgtcctgaggactttacctttcttctttcttgaaaggacctataagactaaatgagataacacatggaagctttgaaactccttaagggaagaaggccttatgaaaagaaatttctaaatttcacatgtgcacatcatttgcttaacttagatcattgaatgattagacatgaaagcaagaaaaaaatgtaaactgtatgaagttgaggtttttattttagaattttagtttcaaggtttaattttctagaaaacttgcagcatctctgagaaatttctgatgatgggaaaagaaaaggccctactcagcaaaatagattaaaaaaagaactgaaacccttaaatttgggggtaaattaacaattttaatatagttttagttgtaatgagaatgtgagtagtttatttcaaatactacaataatttttttatcttataataatgggtattcaataaagattgctaattttaaagtatattttgtttttttctgaaactattaaccttgcttaactcttaaaccttttctaatccttaaattttgaaacatgttttgcctgtaacttttatagttgtggcagagaataacaaatgatttaagcatttaaaatacatattttcttttctttttttctgctccagtcaatgaaagggacactttattgaggccccagggccatggggcctgggcagaaggctgctgcccttcaaggaaggaagagccttatttgaccttcttcctggggtacaggttgttggtgtggccgcatttcttcttgtggcagttgatagcaccggggtgcaggcaagcatagcacttgcagcagatcatctcgtcgcagttgtatttctggacgagctggcggagggaaggctcagtaatgccacctcgcaggcgcagcaccaggtttagggtggactctttctggatgttgtagcctgagagagtgtggccatcctccagccgtttcccagcaaatatcagacgctgctggtcaggtgggataccctccttgtcctgaattttggttttgacattctaaatggtgtcggtgggcttgacctcaagggtgatggtcttgcccgtgagggtcttcacaaagatctgcatctctgcgtctgcagcccggccgcctcgctgaagaaaaagagctaaaagacatattttcattgaaaattatttacttactttaaaaaactcttaaaagtaactggtattcagcatttaatgatgatatttagggttttcaaacccatttttcttagtccatttgggctgctataacaaagtataataaactggatttttataaacaacagaagtttatttcttacagttctggagactgggaagtccaagatcaaggtgacagtagatttggtgtctgggtagggcccactttctggttcatagaaggcacctttttgctgtgtcctacatggtggaaggggccagggagctcttgggggtctctttattttattttgagatggagtttcactcttgtcgcccaggctggactgtgatggcgcgatcttggctcattgcaacctccgcctcccgggttcaagcgattctccttcctcagcctcccaaggagctgggattacagatgcccgccaccacacccagctaatttttttgtatttttagtcaagacggggtttcaccatgttggccaggctggtcttgaactcctgacctcaggtgatccgcccgccttggtatcccaaagtgctgggatttcaggtgtgagccactgcacccggccctcttttatattttatttatttatttatttatttatttatttttgagacggattctcctgtccaggctggagtgcagtggcatgatctcagctcactgcaacctctgcctcccaggtttaggcaactctcttgcctcagcctcccgagcagctgggattacagatgtgtgccaccatgcctggctaatttttgtatttttatcagagatggggtttcaccatgttggccaggcttgtctcaaactcctgacctcaagtgatctgctggcctcggcctcctaaagtgctgggattacaggtgtgagacactgtgcctggcctggggtctctcttttttttttttgagatggagttttgctcttgttgcctaggctgtagtgcaatggcacgatctcagctcactgcatcctctgccttccgggttcaagcgattctctcgcctcagtctcctgagtagctgagattacaggtgcctacgcccactcccactcccagctaatttttgtatttttagtagagatggggtttcaccatgttggccaggctggtctctaactcctgacctcaggtgatccacccatgtcagcctccaaagtgctgggattacaggtgtgagccactgtgcctggctctggggtctcttttataagggcataaatcccattcacgagggttctgtcctcatgaagtcatcacttctcaaaggccttacttcctaatactaccatgttgctgattaggtttcaacacatgaattctgggggccacaaacattcagactgtggcaacacttaaaaagttatttgaatgaatgtgtaagacttactttttatttcttggacatgttgctatttctacagtcatttgactacatggttaatagaggaatttggagagaatattgcaaatgtgacttcaagggtaaattatttcaagtaggtatttaagaaacaacgaacttaatacttgaactatgtgtacctttttctagggaacctcaagttaaatcagcaaaattcagcagggagaaatgaactgggttatccatgtctcaaaggtacaaagaaatagaacagtcctctcctacctaccatctctgtgtattataaagtcaaggagattattttacttatggtttaatttaaagagacttatttacaacatgtatttatatgacagtttgatatgctttatttttctgcaggattcaattactggacttgtcaactctgccagtgtacgtgccatttctcttccactatgagaggaccgattgtattgcacatttgtctggctttctgtagccttctgcttttcagcgttgccacacaatgtctggccttccccaaaatagaaaggaggagggagatagcacatgttcatgcggaaaaagggcagtccgataagatgaacaccgatgacctagaaaatagctctgttacctcaaagcagactccccaactggtggtctctgaagatccaatgatgatgtcagcagtaccatcggcaacatcattaaataaagcattctcgattaacaaagaaacccagcctggacaagctgggctcatgcaaacagaacgccctggtgtttccacacctactgagtcaggtgtcccctcagctgaagaagtatttggttccagccagccagagagaatatctcctgaaagtggacttgccaaggccatgttaaccattgctatcactgcgactccttctctgactgttgatgaaaaggaggaactccttacaagcactaactttcagcccattgtagaagagatcacagaaaccacaaaaggttttctgaagtatatggataatcaatcatttgcaactgaaagtcaggaaggagttggtttgggacattcaccttcatcctatgtgaatactaaggaaatgctaaccaccaatccaaagactgagaaatttgaagcagacacagaccacaggacaacttcttttcctggtgctgagtccacagcaggcagtgagcctggaagcctcacccctgataaggagaagccttcgcagatgacagctgataacacccaggctgctgccaccaagcaaccactcgaaacttccgagtacaccctgagtgttgagccagaaactgatagtctgctgggagccccagaagtcacagtgagtgtcagcacagctgttccagctgcctctgccttaagtgatgagtgggatgacaccaaattagagagtgtaagccggataaggacccccaagcttggagacaatgaagagactcaggtgagaacggagatgtctcagacagcacaagtaagccatgagggtatggaaggaggccagccttggacagaggctgcacaggtggctctggggctgcctgaaggggaaacacacacgggcacagccctgctaatagcgcatgggaatgagagatcacctgctttcactgatcaaagttcctttacccccacaagtctgatggaagacatgaaagtttccattgtgaacttgctccaaagtacgggagacttcacggaatccaccaaggaaaacgatgccctgtttttcttagaaaccactgtttctgtctctgtatatgagtctgaggcagaccaactgttgggaaatacaatgaaaggtaaaagaaagaaaaagaaaaacaaactttggtttaatttggaaactttcaattttttttttaatggcaatttagaaataggttgaagttttaatgaaaagcaggccaggtgcagcagctcatgcctataatcccagcattttcggaggccgaggtgtgaggattgcttgggcgcaggagttcgagaccagcttgggtgatatagtgagacctcttctctgaaaaagaataaaaaaatgaaatttaaaaaataaaaagaaaagcagaagcatacaaatacatcatctattttatataaaaattacacccaattcctgtaattccagcactttgggaggccaaggcaggcggcttacttgaggtcaggagttcgagaccagcctggccaacatggtgaaaccccatctctattaaaatacaaaaattagctgggtgtggtagcaggtgcctgtaatcccagctactcgggaggctgaggcaggagaatcactttaacctgggaaggcggaggttgcagtgagccaagattgtaccactgcactccagcctgggtgacaagagtgaaactccctctcaaaaaagaaaaagaaaaaaaaattacacccaaatttaaatcctgcctatagaccagtgttctttaactggggatagttttattttgctttccccttcccccaggggacatttagcaatgtttggagacatttttagttgttagaactgaggggtgctaagatattgttaaatatcttagactacgtaggacagccccccataacaaagaattatcccagccaacatgttaatagcgctgaggttgaaaaaccctgatactattatttttattttagactaatatttttcaaacattttactatgaaaatgtataaatatacaagaaaatgtgaaagaattagctatatgtttaaactgtaataatgttggatacttacatacacattgttgtaaaatatgctcacatcccttacatttcttacaaaaaaagctttaggtaagtactgcagttatctcatagtattaactatatgaattttctaactttgttttcctaaaaagaaaacatttagaaataaggaagggggtagggtggaagataaatcatctgcttgcttggttctgcattttctactttcttttggcctttgcatccgtgaatattataagctggaaagccttctatttgttgttgatggataatggttttcttttattatgggtagttggacagataacaacattgggaaggaaaagcttagtgtaatgtatacatgctcattccaaattgttttggaacatgtacaacgtgttacttcagccaagtagcccaattaatagaaaatatcaaatcagtaatatgttttataaggtgttgagattgaattaaaatggtgaaagatcacttgataaatgacaaaagaaagattgaattttacatgtctaattcatttttgtttaaagacccagtgtgctgttcagttttcatagaagcttccataaaatgtcttgtaagttggtttttacatggagttgggtgcacatggccatgaagaccttagatatgactgagcaacaagtttacttcagttcttcatagaacttttacttttggagcattaagtcatatagccccagattgttcactgcacaattgaatccatgattcttatgtactaaacttgtattagcttctattagtacagcctaaaaagtaaaatggaattggtaatttcctgattattttggtttttgaatatggttcttagtcataattgcctcagcaaattataatcttttaaaaggccactacttttaatttctagcacatagcacaattcctggcacatagtaggaactcgataaatatgttttcaattaatttacaaatgaggatatagaaccattcagactggagttatccaatgtgattgcctaccttacccaaagtcaatcaaacaacttctaaatgttctttgtaccaagaggaacagtcaagttattacttttgttttacttttttttttgtcccatagagttaaaaggaatgggaaagattgcaattattgacagcagttgttatacaatagccaaaatgttttactactcattttcaggtgatcataagtatttttctgaagtaaacaacttaaatgctaatcatttagagtccagggaccacttaggacattatcttcttcagaatatgcaatgatttcttggtgtttttagatattattaacagatgggtatgttgagatgatagtcagtcctttgagtcagttagctgcttcaactgaagatcaggttttaaaaataatatataattattttttctcctacatgaagtaggaagaatgagagatgttaggaaactttaaagtttattttaaaaaattttattactatttctagttctcacttttctaacagcaattatttattcaagatactatgcattttgacatttcttgctatttcataaaattacaaatagcgcctgcatatttatttttgtatatatgttctcaaaagtgttcattatgctttcaaaaatgagggcaacatagagaacacctactgcttaaaatggtaaaactatatgttattgtcttcttgtggcaaacagagcattgccctatgtgctagaggtacagaaagacaatgaatcctggctctctagggatttacagtaatattggagaaaggaaaaaaagagagacaaaattcaaacaatggtatagagtgcaaatatgaagctcatgtgaaaaccttaccctgactcacattgtttgtcctgtaaccaggtatattaatccatcaaatgtaaaaatctcttcaattcaaaaggaaaaatagtgttaactggatgagtaccaagaaaaggaactgcttatagtgaaaaacaaacaaatacacattattttaaagcaaatacattgtttacaaggcaacttttttttaaagtaataggatatgccatgatgtcattaataaattttttttttttttttttttgagacagagtctccctctgtcatctaggctggagtgtagtggcatgatctcaggtcattgcaacctccgcctcctgggttcaggcaattgtcctgcctcatcctcccgggtggctgggatgacaggaatgtgccactatgcccagctaattttgttgtgtttttagtagagatggggtttcacaatattggccaggctggtctcgaactcctgaccttgtgatcctcccgcctcagtctcccaaagtgctggtattacaggcatgagccaccgtgcctggcctgtcattaataaaaactttgaggaacccttttaaagctatccctagaatgttatttttcattgtataacttactgatcttgctaaaatacttagtatctagaactaatattttaaaaaagacaataaggttttcaagaaattaagtttttagtagttgctctgtacagtgtttgaaatttctagcatagccccttcataagagggaaaatcttgacttataagtagtctgttgaaggcaaaacatttatatgatctgaagagaaagcagagtattattataaacagtctttaatactgtatttgagggaattataatgttgtaaagcaagtggacctgtgacagataatgtcataccctgccagttctattcatttccttttctgatctgaaagcaaatgcacaccttctacagaggcctctccaaggtgttccttgggcatagttagtacagtggttaagggtgctggcaatgtgaacaaaggcatgctccttagcttttctaaacctaagttctgtaaagtggagttataatagctggtaattattgaatacttaccaaatactaggtgttttacttgtgttatctaatctttgtagtaactctgtgagatagatactattattatccccattttatagctgaggaaactcaaggcccacatatgtgaagtaatttgtttatgcttacacagctagtaagtgactaaggcaggttggctctggaacctgcattcttgagcactatgatgcagcttctgtggaatgatattagtacctatctccctgtagtacatatgggaggatgcatacttaagatactatatgtcatgcacttcgcatggtgtttaacatagaaactcagtaaatattagttattgtaattattactcctttattttaaaatgtgagcttcccttgaccctccagaaatgcaaatgtgaaaattaatacactgtatacaaatgtttctgagagtttttgagtgtaacttagttctgtataacttgtttcttaacaaactgattatgttgacattcaaacatgtgcctgaaagggagctcagcaattgatcttcctactttgttttagatttcattttagctttttccatgaacttctttatctctgttgtagattgattcacaactactgaaagcgtgacagagaaaacatttatttactgtcattttacttctgagttctctctttgcttccccagttcctgaaatagcatctgctcccctccccccaagtttttaatcatatcatgattttttttttttaaaaaaatagggatttggtctatttttaaatgttttcccccctttaagacatcatcactcaagagatgacaacagctgttcaagagccagatgccactttatccatggtgacacaagagcaggttgctaccctcgagcttatcagagacagtggcaagactgaggaagaaaaggaggacccctctcctgtgtctgacgttcctggtgttactcagctgtcaagaagatgggagcctctggccactacaatttcaactacagtcgtccctttgtcttttgaagttactcccactgtggaaggtctgtaagggaggatattggactctgttttgggggtggatctcttctgttaagtggactaggaatagaatgagtgaggcagccgacattgccatcagcgcgcgcgtgcgtgcacgtacacacacacacacacctttacctggaaaggggtgtgagctctgtcctgtttgaagaggtgtggcagagccttaaaaggacaagggttggccgggcgcagtggctcacgcctgtaatcccagcactttgggaggctgaggcgggtggatcacgaagtcaggagattgagaccatcctggccaacgtggtgaaaccccgtctctactaaaaatacaaaaaattacccaggcgtggtggcgggcgcctgtagtccgagctactcgggaggctgaggcaggagaatggtgtgaacccggggggcggagcttgcagggagcggagatggcgccactgcactccggcttggtgacagagcgagactccgtgccaaaaaaaaaaaaaaaaaaaaaaagggacaagggtttagtttgtaagtaaggtctcctggtggctcacctctgggtcctcaaaggccagaccccctgagaaggctcttcctgtttggcaactctgctgctaggagccgggggggatgaggtgaaaaaagcatggacctgaatttgaatcgcaactttggctttcactaatattgtagcctcaagctaagtttcttaacttctgtaagctttggtttgttcacctctaaataatgcctatctgttttataggttattttaagaatttgaaatcatgtctacaaagtgctttctatataggaggctctcaaatgctctgatctcttaaaggcagtgaagttttcaaatctatgtgaaagctactgctgttctagagtctctggacttgggaattcctggggctatatttggctgcttttaaattgacattccagacccagatctaggctcttactctatgacttgattttgagaaagacactcgatctttccaaagtagtctttgaaatacagtttaatatatttttgttaattattttacctttggcaatacataatataaaatcttacaacatccataatattgccaatagaataaatgggagattaaaatttaaaaacaaagttctgattagagataatacattttcctgtaagacaatacattttcctgaaagtctttgctttggggagttggattaatacaatatgggaaaggagagctgacccaagtgccaaacttgctgtattgtatgatattggtttaatcatttcttgcctaaaaagtgaaagcacatgcttacatttgccataatgattatcttgtgggtcaagtcctgtctgttgtgtttggcagaacaaatggacacagtcacagggccaaatgaggagttcacaccagttctgggatctccagtgacacctcctggaataatggtgggggaacccagcatttcccctgcacttcctgctttggaggcatcctctgagagaagaactgttgttccatctattactcgtgttaatacagctgcctcatatggcctggaccaacttgaatctgaaggtattcatgctttgaaggggatcttttcaaggactggttgagtgagtcactgagcaaatgtttttgaaaaagaattaagtaggagtatattcaatctgcagtatagatcagaggacagtaaactacagctgggccaaatctggcctgccacttgtttttgaatggtttgtaagctaagaatggtttttacattttgaaatgattgaaaaaaaaagtcaaaagaagaataatattttgtgacatagaaaagttataaaaattcaaattccagtgttcataaataaagctatattggaacacagccatactaattcattttcatatgtgtgtggctgcttttgtgccatagcagcagaattaagaagctgtgacagagactcagtggcccacaaagcctgaaacatttatgatagggccctttatagagcaagtttgctgactgtgctatggatctgtggttctcaactatagttgaccattagaaataccttggagagcttttgaaacatcccagtgcctgaatcactctccagtgattgagattaagatggtggggggtggaatctggcatcagcattttaaaagactgccttagatgatttcagtattcaagctggattgagaatactaggttaagatggagatgttgagtttccccaccagattttcttggttgcaagttttaatgtggggtgtattttaacagcattgctctggttgttaatgctgggttattctcccttagtaacatctctgggttagtctgtgctactatttggagataggcagtctctttatttactaagaccaagaaattcatgccaaattgtactttccaagacattggctttgagcatgtaaaagaaacttgaggcacggctgcctatcaacaaataagaagactagaaatatgacacatagtcacgtaattaatcagcattttggcaggtgaatagttcctggctggagatacaaaatgtccagagatttatagtgcagaatttcttaaactaatctggaacactggtaagcttttgggcactcaattagccatgtgtttgctgcagtaagagacataggatgtgctattatttttttttgtctaaagcgaagggaaggaggttgagctaggtttgcagctgactctggtgcagactttctgggtaacactggttggctgctgacagaggaacatgacattttaggagcagtgtggagaattccacagtagaagcctggtttaagaaacagatcaaagtgggctcaaaggctaacagctcttgcattccacattaagataaatactgtatgactttgagtttagcgtctcatataaactgtctgctctccttgatgccatctggaaaaagcaggtttccctaaattaacctgagttagacatgacagctttacccagcagggctcattaaatcagagaaaaaaaatatttgtgaataatccatacttgtgaaactgcagtaactttctaaatttagtagatgatggcagttgtattgaaatatgttagaattttccgacatgcctaaggtgtcctggggttttagttcttttcttctacacaattcaattttgctactttttgataatatattttgttagtttttgatctaaattatccttgtcatgggttacaaaaatatttttggtcaaaagatattatttttgctacattgtcgggcttaggattttgaattagcacatttttactcgtaaatgccaaggagtacaacttatgtttaattagtatacgttaataataccattgttagttttatgctggtgcttctagtcctccaaaagctgaattgactacactgatttaaacaaatcaacctactagactcctggatgtattggaggcctctattttcaatgcatgtttttctaccttgcccgggaatataagtgcagcttgaagctttgactgggtgaaacattttaattcttccataagcttatgcctagaggcaggtaattttttttcttatctagaaccctgtattacttacgtcaggttagcagcctttcttgttttagtctttgtgatgagtttatatgttattttaatatcttcctttttcactagggggcaggagcatttctctgggaaatgaggtacttttctccatgggatttggaaagggacatcctttgttagacattagacaccaaagaagaatgaacctttatggaaatttctgaatccgcagctctgtttatcctttaagcgataactaacagcccaaatcagtctaaaaatgatgtctacttgcaccctgtcttcttccctgcttctcacatctctacaacatgatgactgagagggggtatgagttttcaagggctgtcataactaagtaccacaaactgggtggcttaaaacaacagaaatttattcttacgcagtctggagcctgcaagttggaagtcaaggtataggcagggcttgctctctccaaagcctctaggggaggaaccccagggtttccttggcttgaggcagcatgactccaatctattcacatggccgtctgtgtctctgtgctcaagttttccttgctttttaagaacaccagtcattggattagggcccatcctaatgacctcatgttaattatgtatgccaagaccctgttttcaaatatggtcacattcactggtactaccggtcaggacttgaacataccattttgagggacacaattcaacatgtaacaggggtctgcttctgaagagtttgccagctgtctgtgaagacagacgtgctacctgagtgatgtgaattggtagagtgggagaagttttggtgtgttttccttttcttagtcactgaagaaagcctgccttgataagtgcctgccaccttgtgcatatttcagtgtcttttatcttgtcagcaaaaatgcagtttgatttttgaagggataatggtttatttttcaccttgcctgttacatttgatttctatgtatgacttttttttagggataaatattctcacaatgataaatgccttatactcatttcaggcatacctctacgagggctgtatttagcagctttgaaatacaaacataacaaacatgttctttttattcaaaatgggataatgacaaattttatacatattttataactgcataaattaatttttggacatgatctttgtataggttgattttcttctcttgttgaatattaatgtctttcctgctctaaaatgccaacttaaaatttctgattagtcttgaaaacatacctggaatttatgtgggttaactctttaaaatattttagtactaatatttcttacctacttaaagaagtcccgctttctgtatgttagaaatctgtctgaagatatctgcttactctaaataaatcaaacttttattttattctttctgtatacccccatggaagatggaaattaatctgtgctctgaagtggtaataataattctgtagaatggaaggttgttgctttggcaattccatattcagaaaaatctactgtgggtctatttgtgtcagatagtaggtttctaagaacatatgggagataagaatataaaaacagagatttctcagatcttgtgattaagtctttgacggtcctccctcccaccccccgcacttcttcccttaatatttggaagtcaatatctgggccttccagctagcgggaaatggggagccagttggtgcttgccatggtgcggaccacacagagacaggaatacagtgacaggaacactgaatttgcttggactcttctgggttagctcttctgcaagaaaaaagcaattccagtgttcctcatctggggaacttgaattcttgatccgattggtcttatttaatatctttctgtgggcatgcatgttgagtgttgcgtataatcccatgatattgtctcaaccctgtattagtctgttctcatgctgctaataaagacatacctgagactgggtaatttataaagaaaagaggtttaataaagtgaaagaggttccacatggctggggaggcctcacaatcatggcgaaaggcaaaggagtagcaaagttacatcttatatgacagcaggcaagagagcttgtataggggaactgccctttataaaaccatcagatctcttgagacttattcactgtcatgagaacagcatgggaaagacccacccccatgattcagttacctcccactgggtccctcccatggcacatggggattatgggagctacaattcaagatgaaatttgggtggggacacaaccaaaccatattaaatccccatttcctcatttgtttcaaaaggcccgcttattatagaccttatgaaacaaactcataataattgacttgctggagaattacctttaacatagagatttttaattcttggccggggtaatcccagcactttgggaggtcgaggcaggtggatcacggggtcaggagatcgagaccatcccagctaacacagtgaaactccacctctactaaaaatacaaaaaattagccaggcatggtagcacacacctataatcccagccacatgggaggctgaggcaggagaatcacttgaacccgggaagcataggttgcagtgagccgagatcacaccactgcattccagcctgggcaacagagcaagattccatctcaaaaaaaaaaaaaaaaaatccaatggagaagctacgtttaactttctttaataacttaattttttaaaatacaaaaccaaatatatcctttaactacagtttttttcttttctgacagtttttgcttttctaagactgttttgtctgtctttatatttagtttttaaatgattgcgtttttgacatttttttcccagccaggcaccatacatggggcagaaagctgtcattattttgccactcattgtttaatctgtttcagagatatccattttcctgccttcatgtgtgcacatgtgtgcatgcatgtgtatgtgtgtatttaatatcatggttgtttttgtcctcagagaacaattccagtatttatttgcagtgtctcttaatttcctctgctgaagagatagtggtgttatctctctttccttatctggggtttctagttctgtccccttctgcggtcttctttactgataacgggaatttcaggagaaagtcatttctgaaaagtgtgccttcattcaggtgactgaatgatattcttgggagtctggtggcctcatactgaagccaatgtctttgtctctttcttttatcatcaatcagaaaaaaattgggaaatacattgtttaagtagcattagggtgaagtttttaagatttttttcttttgtttttgcagttgcaagatttaatagagggaagacagagctcccatacagagggaggggacccaaagagggtagctgttgccagctcgaatgcctgggtttatatcccgatcattgtccctccccctgtggtctcaggtgatagatgattggctatttctttgcctcctgttttagcctaattagcgttttagtgagctctctttactatctgactggtcgggtgtgagctaagttgcaagccccatgtttaaaggtggacgtggtcaccttcccagctaggcttagggattcttaatcggcctaggaaacccagctagtcctgtctctcagtccccgctctcaacaggaaaacccaagtgctgttggagaggatggctgacgaccgttctaactgcttcctgctgaattggggcatagtaggggttgtgcagttgagatttcctcgggaggagtgcctttgatgtcattaacatcagagcattggctagcaggccagtccaggggtctgtggcagattttagtcatggactgtatctggggctccatttgaagaacgattggtagttttacagctttgattctggaagagacaaacttaacaaggagattaaagatacagggattgaaatgtatggcctgcagtgcaggggattatttctttggcatacttcacaggccctgactatctgcttgatagttttgaaaaggcctggtccagtaaataataatttggccatctgatgggtgctatcaatgcctaactgaaaggtttggtgaaggtttttaagtaatttctattggttagctgcaggcaaaagtatttttccttcttcggtggctagccatctggagaggaggaaagtatgtcctcatgaggttccctattctatttttcctgctgagtagtggggcttggtttctcggaggggattaccccatactaggggtccttctataagcatttcaaatggagggtcctgccttgtggctcttttggcttcaatgtccacttggtggtttccttctattttcctttcctttcctttctgatgaccccggcagtgtaagactgccacctctttaggtttctgtacagccaataataatctcctaaagacttcctgatgtttaataggtgttcccttggaagttaggaattccctttctctccatattgctgcatgggcatggaggactaggtaagcatacttagagtctgtatatatatttaccttttttccttctcctaattttagtgcccaagtgagggctattagttctgccaactgagcactagttcctggagtgaggggattacttttaagtattccattatcactgaccactgcatacctcgcttttcaaagtcccttttctacaaaggaacttccatcagtatacaagctgaggttgggatcagtcaagggaacctctaaaaggtcccctcgagcagcgtaggtttgagcaattacttgttgacagttatgttctatcttttctttattgtctggaagaaatgttgctggattaagagttgcacaagtatgcagtcacagcactggcccttcaagtaatagagcctgatatttaagtaaacggttgtctgacagccacaagtctcctttagcagtcagtatgccattcacatcataagatgtccacacaataaaatctcttccctgtattattttaactgcttcagatactaagactgctactgccgctactacccgtaaacaatgaggccaaccctttgccactacatcaatttccttactcaggtatgccatgggttgcaagcttgtcccttggatctgtgtaaggactcctagagctattcctgttttttctatgacatataaagaaaagtcttgctctgttggcaagcttaacactggggcttgggttagggccttctttagggcctggaaagctgcttctgcttcaggtgtccatcttactaaatgggtattggctttctgagtttccttaattagtgtatataatggtctggctattttgctgtacctgggaatatccatatttggcagaaacctgttatgccaaggaaccctcttagttgctttagggttttgggatgagggtaagccagtataggctggatacgttcctcaccaagggccttggtacctttggataattttagccctaagtatttaacctgctgtgagcagtgctgagcgtttggtttggaaaccttgtagccacaggtagcaaggaaatttaagagcgcttgggtggcttgatggcacaaggtttctgaactggcagctaaaagtaaatcatccatgtaccgaaggacaagagtgtccaggtatgagaattggctcaagtcttgggctaatgcctggccaaatagatgggggctatccctgaacccttggggtaaaacagtccaggtgagttgagacgttgggtttgaaggatcttcaaaggcaaacaagaattgagagtcaggatgtacagggatgctgaaaaaggcatccttaaggtccaggactgtaaaccacactgcttcctctggtatttgggaaagcagagtatgagggttaggtacagctgggtctagagggacaacggcctcattgataatcctgagttcttgcactaacctccactgtctgctgggtttctgtactcctaaaattggagtattgcaggggctattgcatgatttcactaggccttgggcttttaggtccttaacaatcttttggagtccttgttgagccttgggtctaagggggtactgcctttggtagggaaaggaggcagaatcctttagtttaacttgaacaggacgagcattctttgctcatccatattgtccttctgttggcaagacttcaggattaattccttcctcaagcaggggataacaaatgggtgttccttctcctatgttcaggtgtataatggcccctgcttttgctagaatgtctctccctaaccaagggagtggggctttcaggcataattagaaaagcgtgtgaaaagagtaaagctcctcagtcaaaacttagtggctgggagaagtatctagtgactggctgtcctaggacccctcggatagtgacagatctggaggacagttgtccgggacaggagagtaagaccgagaaggctgtgccagtgtccaggagacagttaacctcctggccctcaatggtgaagcatacctggggctctgtgagggtgatggcatgggctggcacttgccccaggcaccctcagtcctgctgctggatcatctggttagtggtttctgactcagaggaccttcatcccctggggcagtgggccttccagtgattccctcaacataaggggcatggacaagggggcggcttacttctatttggacaatcttttttaaagtgtcctggtagaccgcactggaagcaagccctattaggcattcgatttgcccagcttttcccttttccagagcctccaaagtccacttgcctgagggccatgactaaagcggtggcctttttttttataccgtttgtcctgttccaccgctcctcctgatctctattatagaaaaccaaggttgctaagttcaatagggtttctaagttttgctccgggcctaaggtggacttttgaagtttttttctaatgtctgtagctgactgggtgataaacttatcctttaagattagttggccttcagtagagtcaggtgacagggaggtatgctttctcaatgcctcccttagtctctctagaaaggcagtaggattttcttcctttccctgtgttatagtggacatcactgaataattcataggcttcttcctagttttccttagtccttctagcacgcaagttagcaaaatcccatcagaaggccatatttataaaaccaagtttgagtgatttttcctttggttgctgctgtgtggtatatgatgttaaagatcaagaagagtgtgataggcttgaactgtctcctgctatttgtgattctgtagtgtgagattatgaatgttcactctgtgttaagaatgggcacgagtattggcttttgttcttggaaatgctttggattctgatttcactttgcctcaatgacagagccacttaactgatacacagtggagatgacatggaaaagagtattccatggaatatgcccttcatttgatttgaactcaatgctgggtctttctcccccagttgcctgttctgttgaatgggaattaaataaaaaataattttataactattaacacgaacacatgaaacatctaatactttttgccaaatgtgttagggaaggatgaggttcccttttcttgttttcatttttgtgtcactttaagtgtcttatatgacagattatgaaacacagaattgctggtgatttcagttatcagttgcctcttatggtaagtcattttgttgtcttttcctgaaatcatcgtgctttctttggcatttatgacagagctctattcctaagtacctgcatactctgtgggttagtacatccattacgtagcactcagaatgttcatcacgtttgctaataatgctcacatagcctgtggtatttcaaaaatatttaaaatcaatattgtgaggtattgccataaagctgtgttcacaaattctgctgttgaagaaagataactgacatttactctcgtttttctggtatgctttcctgccacagggaattcatttattggtataattgcaattaagtaaaccttgtagagttggttcccctcatctattttttgtgtgtgtgtgtgccttttttttgccagtccggcactgcttggtaagcttatcttagacactcataaaattatacagaatggctcatgatgatgaaaggatttagctttgtacatattcagtggttttatttattctttcagttatgtgtgcatttaacaagtatttatttggctattcagatgataccaagaataattgggggtggatcttgctcttaagggagcacgagtgctagtagaagaggtaagatagatatctacaaaacagaaatgcaacatataagtgaggactaaactctgatgttttaatcttgaccaaattcctatctaaggggtctggagagccatgccctacaaatcatgaattttcatcagatgggttttatttaaccctgtatatcgtgacttactttccaacttgactctggcataacattatgagacaaggaagaaaatataaatattttaccccaaaacatgtttctttgccatatgttgaaatggccctgcaaagcgtcctttgtgggggaaaatgtgcttctgtaaagaatctccattaacgaagctagatctttttcttcctgaccctcccaatcctaaagagattaactaaaatctgaataggaaacatttgtcacctattgtttctaagggcagccactataagacttcaaaagaacttttgtctccacaatctttatcttaacctgaacattccctttctgtcaatcccaggtcttcctgaccaccttgggcacatgttctcacaacctcctgcgggctgtgtcatgggccatggttactcatatttggctcagaatatatctcttcaaatattttacagagtttgactctgttcatcgacagaaggcagaaaggatcacaaaaaaaggactctaagtaagagtcaggagagaaaaaggactctaagagtcaggagagactttagttccatcctagaaaaacactggattggtgtatacccctgtgtgtctttgtgtgtgtgtgtgtgtgtggcatgggggagtgtttgtgttatagatttgtgtatatatttatagtaatgtgctgtataatgaagttttggtcaacaacacacctcatatatgatagtggtcccatatgattataatattgtacttttactgtaccttttctatgtttagatacacaaatacttaccattgtgttacagttgcctacagtattcagtacagtaacatgctgtacatgtttggagcctaggagcaataggctataccttatagcctacatgtgtagtaggtctacaccttctatatttgtgtatgtacactctatgatgtttgcatgatgacgaaatcgcctaatgacacatttctcagaatgtatccgattgttaagtgatgtacaactgtatatttataattttattgataattgtagaaaaatgagtgggaacaaacctaacaatatggaaaaaattctaaagagtggtgaatagataccatatacaccacactagcaataaattgttttatagccgtgcaatcaaccaagtcatagctcactggttagatgaattatttcttaagaatagcttttaacaactggagcggctgcttttccatgaagaacaggcaatcaggtgctcagcctcaggctaataaatatctagtttacagtaaactagatatttctatagaaagagatacatgtcgtttttgaggatgatgttcagactcctggcaaaaggatttgttctgactgtttaatctgggcacagactccagggttgagaatccctgtgggcagtggtccaagcagggctttccatgtgtggtggcttttgtgcagatgctgaggcaaacacgtagcttggaactatatgtgagtggtagaaatttcaaagatattttagatgcttgcagaaaattaaggcattttcaaaggcatgatttttcaatatgaggatagacatatgtaagtattctttgattggtgcaaacatgagaattttattgcattggaggacaaacaattacaaaccttaaatatgtatagaataatgtgtggacagtatcaactacactttatcaagctgtattcataaccacgaagttgttttgccatcggactacctcttagcatttaaatgaatgtgtatccctgagggaaaagattagaagaaaaatgtaatacaccaatttaaatgaccttacaagatttaaaaattatcacacttggaagcattacattggtcctgaaagatttacattgattttgccctcatttaccagggccttttcttctccagccattctgtctccttctacattgcttatctctagacacaatagtctacagaaatttcttcttctattaaaacagcataaagaatctgcaacgttttagcagcagaaggagattctccaggtgggcaggagcctggtgctgctccctctggattttttttaaggtgtctgcttcacatgctctctgaggaatcctgtttcctattaaaactctggctcaggaagagatgtacatcctttcacttgtttcagctgaaattagtagaaatttttaaaagacctggctcaaacctaagggaacacaaagcaaataaactgagaagtgaaagaaacagaaaatgaattaggttcgtctggtacaaatttctctctgatcttcactaatgagtttctagggattatgggccctgacccattccgtgctctgtctgagtgttttatattctgttcccaaagcactggcacaatatttcaggcactttgccaggctttctggcacctcagtgcttggcttttttttttccccctatgtaaagggtaaacttctcattttctactccccacccccgcccccgcatagaagagccaagaccaagatatgaatttctcctagggctcaccaccaagtagtagtgagcccctaccccctgcatacgtactgtctgtcctgtttacaggttgaaggacacacatttaattcctaggcaagtgacagtccaacagttgtcatgattaataccatgtacatgccacccttcagagaaaattccatcccagttaaaggtatgattcatttccaggcagtatagcactgttctcaccacacaagaaatacacccatatgataaaaagatcattctgtcactttctatagcaaataatactcagagctcctctgctccataaacagggagctatgaagttaagaggccaggaaagaaacagaaagaattcagctcaaacttattctcctctatgaagcctcctggaccactcagcccacactgatgagtagaataagtgagtggaaaacggctctctcccagcactttgggaggccaaggcaggaggatcgcttgaggccaggagtttgagactagtctgggaaacatagtgagactccacctgtataaattttttttttttttaattagctgggtgtggtggcatgtacctgtagtctcagctgttcaggaggctgaggcaggaggattgcttgagcccaggagttcgaggctgaagtgaactgtgatcataccactgcactccagcttgggtgacagggtgagacctagtctctttaaaaaaaaaagttatactcatttgaaacatatacacgacataacttaatgaaaattggattggagttctaaaggcatttaaatatttattttgaagaggaagatagggtgttgttgagtttatttccattcaataaacatatactgagtgcttattctgtgtgaggcacaatgcttgatcctgggaagtggggcacaaagatggattaaggtgaaatacctgctttcaaggagctaaatgcatgtaagtatgataaaacaaatgcatatttccacaaattattatgtaagcctcattttagataggttgagacatgctttacattttgaaggggaaatctactttcatacatatttttgcatagtcaataatcaacctaaccaacctataatttatcttttgagtatatgtggatttttatatacaagttttgtgtatgtttgcttaaagcaagtagtcctttaaggtgtctaattagctgaagaaaaagatattattccagatggctcagaatttacagatataatattaatatagctttgtgtttctctttatagcatacttgagaaataggattgctgtagtctcgttttctagagggaagtatatgaagctgtcatctgaatcataagtgagttatttaatgagcagatgttaagccaggcacagtggcttatgcctgtaatcccagctactggagaagtggaggcaggagggtctcttgagcccaggagttcaagactgcagtgagctgtggttgtgctgctgtactccagcctgggtgacagagcgaaactctgtctctaaaataaaggagcaagtattgaactctagaacttgtaattattgctacagggctctaccagtgtaactttggggcaggatttatagaagataatggataacataccaattatatgggctcatggctgaaaaggagttgttgtgggcagtaaatgcgaaggtacttatgtaaatcggtatcttcagcttgttaaccagatataggcaatgccattgtcaagagcagaatcctcaaattcagagaaagtaatactaaatgaaataagaaaattatttatttatttgtttattgttgagacaaggtctcactctattgtttaggctaaagtacagtggcgcaatcatagctcactgcagccttggcttcccaggctcaggcaatcctcccacttcagcctcctgagtatctgggactagagatgtgcagcaccccacccagccaacttttgtatttttttgtagagacggggtttcaccatgttgcccaggctcgtctcaaactcctgggcttaagaagtctgcctgcctcggcctcccaaagtgttggaattacaagtgtgaactcctgcacccagccaagaaaatatttaaacagctgcctgaaatggaaattgaatccaaaacattttgccactggtgtttgaagtggactatcttgtctcaagcctttttattttctcatgaatgtaccttctaaatattgttaaggctacaatggaggaaattttaaattattatatttactattttacagtgagtggaaaatttgaaatttagtaaaaagttttcaagaaatttttatagattaataatggagtttaaaaagtcatggcgatgaggcatttaacaaggagagaactgaataaattaacagacatgtttaaggtaatgtctatttttttctttcaacttttaggtttgggggtacatgtacaggtttgttacatgggtaaattgtgtgtcactggggtttggtatatgaatgatcccatcactcaggtagtgagcgtagtactagatagtttgtttttcactcctcttcctccttccaccctccaccctcaagtaggccccagtgtctgttgttccattctttgagttcatatttactcagtgtttagctcccacttacaagtgagagcatgtgatgcttggttttctgttcctgtgttaattcacttagtttgatggcctctaggtacatccatattatggcataggaaatgattttttatggctgcatagtattccatggtgtatatgtatcacattttcgttagtctaccattgttgggcatcttcgttgattacctgcctttgctattttgaatggtgctggtatggacatatgagtgcatgtttttttggtagaattatttgttttttttaggtgtatacccagaaatgggattgctgaatcaaatggtagttctgttttaagttctttgagaaatctccaaactgctttccacaggggctgaattaatttgcattcccatccacagtgtacaagcattcccttttttctataacctcaccagcatctgttatgttttgacttctaataataaccattctgactggtgtgagatgacatctcattgtggttttgatttgcatttctctaatgattagtgatgttgagcattttttcataagtcttcttttgagaagtatttgttcatgttctttgcccatttttaatggagttgtttgttttttgcttgttgaattgtttaaatttctcatagattctggatattaaacatttgttgagtggatagtttgtgaatattttctcccattctgttggttgtctgtttacccccttgatagtttcttttgctgtgcaaaagctcttaagtttaattaggtcccactcattaatttttatttttgttgcaaatgcttttggggacttagtcataaattctttgccaaggccaatgtccagaatggttttttctagattttcttctaggatttttatagttttatgtcttacatttaagtctttaatccatcttgagttaatttttgtacatagtgaaagttagggggccggtttcaatcttctgcatttggctagccagctatcccagcatcatttattgaatagtgagtcctttctccattgcttgttattgttgactttattgaagatcagatggttgtaggtgtgcagctttatttcttggttctctattagaaggtaatatttaataataacatttcagatttgatctaattttaaaatatacccagattacagattctcttatgagtatttgaaattggagaatttaaaaatggagtttgaaaaacaaataaaaactctcactcagctcgttggacctatttacagtagataaaatggatgattttgagtatggatgcccttgaaaagtaccttatttggatttcagattctgacagaaaactaatgggtcttgacatagcaaaatgactagatcttgggtaaaacatatgccttaataaattgatgggattacaagtagtcagaaaaatcttcaaggattgtgctttctggcttggtgtattccaaagatgggctgaaacccaaggaattaactgcaaggaagaggtaacggtgctctaggacaagtagtgatatcattgttgtgctaggtgtctacacttgagccagtagtaagatgaggaacctggactaaaatttccacattaagcctgtgtaaggggaataaaataaagtaattccaggtttgtatcacaccctgacttcccacagaaggaaatgtaaattctctctggaaaaaatcatgtccaatagagtcccataggtttcccacagattagaaccaagcaaatgtgaggaataaaataaatgtgttgtaataaaatatcaacaaattaacaaggaaatgaaccaccatgactgacagtcagcagaaaagacaaagccaagtaagattggcttactctttactcaggacctttgttactaaaactatccagacatagatataaaataaccatttataaaagctttaaagaaataaaaaaaagtcacaaaaatgaggaggtaataaaacactatcaaaaatgacccagcagattttaaaaaataaccaaatagaacatttagaaatataaatataatttagaaatataaatataattttttgggaaaaaagcctcaatgacaggttaaataatccaaaagagaaaattaatgaactggaagatcaatttgaagacattatagagagtgtagcacaaagagacaagtgagagaacatgaaagaaaagctaagctttatggagggtagagtgaaaacttctcacttatctaatcagatatccagcaggagaaaatagagagaatggaagagatacggagataacagagggataggattcatagtggtaatggctgaaaatttttccaaaactgtgataaaatatgaatccatggacataggaaacacatgtatcacacaagataaataaaccatcaaaaatgcaaacacaagcacaccaaaagatgtacaggagtgttaacagtaacactgattatttgaaaaagcaaaaatcttgaaataacctagattctaatcatcaccagtagaatagacaaataaattgtgctatgttaatacaatggaaaactacatggtgctaaatatgaatggatgattgccacatgcagcaatataggggttgatcttgcagtgaaagaagctagacactaagaaatgcacacaatataattccatttatagaaatttcaaaaaccagcaaacctaatccgtggtgttagaagtcagaatagtgcttacctttggggtaatgatggtggtatgaagggggcttctggtacaatagaatgttgcttttttaaaaaagaatatgggttgtgaatatgctggttcatgatgtaatagtttattaagttgtacacttatggtttgtcaactttttgtttgttctacatcaataaaaaaagttaaaaataaaaatgctcaacaagatacattgtaatgaaatgaaagaacaatgaagacaaagagagaaatctgaagatccagatttaaaggaaaaaagccaggggaaaaagattacctacaaaggaacaaaatttagactgagagcagacttagcagcaatggtggaactggaaaccagaagacaggagaatctctttaatgaattaagagaaattaacgaataacctagaaatatgtgcctctacttttaaagaacaaaggcaaaaatactggcaacagattttagaaaaggaacttatgaaggcattgcatttagagaagaagaaagataattctagaaggagggactgagatgcaagaagaaatagtgaacaaataaattggtaaatatgtacataaatcataagcactctgtataaaatataatttctaatttatggagtaagaagagaggacagagctaaaatagtggacaataaaataaattgggaagatatagccagggttaaaggactctaaagcccttcttattgagtgataagatagtgattaagttcagtctttggttagttaaatatgcgtggtaaaatttaagagtaacagctgaaaaaatgcagtaggcagtagaatgtataatttctaaaccagtagtgggggaaaatagaataagaaaaaaagccaaaaaaaaaaaaaagaaaaattaagcacagaaaaagtggaaaaaataaagtaacaaacacaaatatatcaataatgacaataaatacaagtgggctaattatgatgttagccagaggtttcttgttgacaccctttatcagtgttatggactgaattgtgtccgtagaaaatttatttgttgaagacttaatcactaatgtgattgtatttggagacagggtctgtaaggggatagttaagttatatgaggtcataagagtggggccaaatccaataggactggtgtccttataaaaagagaaagagggctgggaggggtagctcatgcctttaatcccagaactttaggggggcgaaggcagaaggattgcttgagaccaggagtttgagaccagcctggtcaccgaagagaccctaatccctacaaaaaatgaaataaattagctggagatagtggcatgcacctgtagtcctagctactcgggaggctgaggtaggaggattgcttgagcccaggagttcaagactgcagtgaactgattgtgtgattgtgtgactgcacttcagtctgggtgacagactgagaccatctctaatgaaataaaaaaaaaaaagaagaaggaagaagaacaagagacactagagtctctgcaagagaggaaatgtcatgtaaggacacagcaagaaggtggccatctgtaaaccaggaagagaggccttaccagaaacccacctggctggtgccttgatcttggacttctaacctccaaaactgtgagaaaataaatttctgttgtttaagccacctagtttctggaattttgttatgacagcccaaacttactaacacaatcaagttgtcccttcttagtttgctgaaagcttagattttgtttttttctttttggacattgtgtgcatattggatttttatcaaatgctttttctgcattggtatttctgtaccttttaagatgatgatatggttttcttttttaaggagttactatattgaattatactcattggtttttgaatactaaaacagctttatattattatatattatccttcttatgtattgtattcaatttgttaaaatcttcaaacaaaataaaatttgagacaaaagcattccttgacatatataaaatattgtctccaactgttagcaaatatgttatcttcaatcacacatgaaatatttataaaaattggccataaaccagtccattaaaaaaatcctggctgggcgtggtggctcacgcctgtaatcccagcactttgggaagctgaggcaggtggatcatctgaggtcaggagctcaagagcagcctggccaatatggtaaaaccccgtctctcctaaaaattagctgggcatggtggtgtgcacctgtaataaaaaaaattagctgggcgtggtgtgtgtgcacctgtaattccaactacttgggaggctgaggcaggagaatcacttgaacccgggaggtggaggttgcagtgagccgagatggcaccattgcactccagcctgggtgacaaagtggactccatctcaaaaattaaaaaaaaaaatctcaacaaattttaaagaattgatatcatgcaggctgtattctctgatggttgtgcaattaggttagacattaataatgaaaaagataaattgccccatacttttgaaaatgaaaaaaactacttctaaataacttgtggatcaaagaagaaattataatcgaaaatcaaaatatttggaagtgactcagagaaaatatatcagacttgtggggtgaagatgaaggattatataatgggaaatttttaacctttagaaagctccctcagtgagtttgttcactttaaaaaataaaaaagtttctgttattgtataacatggtgactatagttaataacaacatgctgtattcttgataaatactgagagtggatgttaaacgttctcagcacagaaaataactttgtgaggtaatgcatatgttagttagatttagtcactccacaatacatatatacttcaaaacatcatgttggacatgataaatatatacaattatcagcttaaaaaataaaacccaaaagcttagaaaagcggaaaggatgaaaattaatgagctaagtaacttccaccttaagctattgatacggtttggctctgtatccccacccaaatctcatcttgaattgtaatcccccatgtgtcgaggtggggacttagtgggaagtgattggatcatgaaggtggttttcctgtgctgttcttgtggtagtgagttctcatgagatctgatggttctataagtgttggacagttccttcttcacactcactcatggtctttctgactcctgccaccttgtgaagaacgtgcctgcttccccttccaccaccactgtaagtttcctgaggcctctccagccatgcagtactgtgagtcaattaaatctcctttgtttatgcattacccagtctcaggcagtatctttatagcagtgtgagaatggactaatacagctataaaaacaaaatagaagaaacccaaagaaaatagaatgaagtaataacagtaagatttcattattttatttttacattgcattaattcagaacagcttagtaaacttttgataaaatttatatggaagagcaataggccaagaaatagccagtgtactcctaaagaagaaaaagaaattgaaggaaggtgtctgtaaataccctaacagatattaagacttataaagctcttactatataattatatatttggacaaggaggacatggacaggaatagtcaaagtggccagtgaagcagaataggcatggaaacacactgctatagagggtccttgacatgtgacgaaaattgcactgcagagtgatggggaaagcatttaaaacaaacaaaaagttctgggacaattggttgtccatatagaaaaaactatattgaagctacatcctacacaaaaatcagttctatgtggtttaaaaacagatgtaataggcaaaccttaaactgactaaaattgtagaatatttttctgacctcacaataaagtttttttagatatgacacaaaaattgctaaccataaaaatcctgataaattcagtgatattaaaactaaaaccttttataaaaggatgctagaaagaaagtgaaaatacagcttctaaaattagcaaaaatatttgtaatccatataactgataaaggattgatgtccaggatagaaagaactctaaaagaatcagcggaaaaatgacagacagcccaatagaaagatgagtgaaagacatgagtaggcatctcacacagatactgaatggatacacaacctcattagtcattaagaaagtgcaaatgacatctggagtgacatcattttatacacactggattgaaaagaaatgtttgacagtgccaaaggttgatgaggatgtggagctagggacatttatctagacattaggatgttgaacacttgtattccttttaacctagcaattccacttttaggaatgtaccctagacaaagccttgaacaaatacactagagatcatttatgagtgttcaagcagcactattcataacagcaaaaaaaaattagcaacaaccaaatacccgaaatcttccttgacatggggataaacaaactgtgacatatatgcataatgaacagcatatatcgtgaaaattaataaactagtgttttgtacaacaagatgtgtgaaacttaaaaccataattttgaatggaaagaaagtccccaaatatagcagtcagtaactttgtatggctttaaggggaagaaggatgttaagaagaatatgcagataggtgtaacagtattaataacttactagtccttaaattgaatagtgagttctttggggttcatttcactatgaacttaataagataaatacggtatatgtatatagtctaatatatatctcctattacatgataaaaatgtaaaagcacttggcaggaatatttccattttagaaatgtaatgaaattctatgtattctaagtcagcttaactgtgagttgctggattttgtggtagttatttcagaaaattctggtatcccttagaaccttatgatcctggagagccatctgctggagagtcgctgcacatcctcaagacgaatttaatattttctttctggcttttaagttttgcacccaatttgatgcccaaggtaaaaaaaatctggaaataaaagcagtgtcatttctagggcttcagtcactttataggtggaaacaatggcttttgattgttattggcataagagttcccaggaaaacgtatgcctacaaatgtttcttctttgagaaagatgccaccgggatgtgtcagagccattcccttcatgggagaagtgggagaaaggctaatatgcgctcagtccatgcctggtgaagctaccttgtttcttttcatggcatccttcttcttttccacatgggctaattttttttttttttttttttttgagatggagtcttgctctgtcgcccaagctggagtgcaatggcgtaatctcagctcactgcaacctccgcctactgggttcaagcgattctcctgcatcagcctcccaggtagctgggactaagggcgtgcaccactacgcccggctaaattttgtatttttaatagaggctgtttttcaccatgttagccaggatggtctcgatctcctgacctcatgattcacttgccttggcctcccaaagtgctgggattacagccgtgagccactgcatccggcctctaatttgtttaccttagatttaattttaaacttagtaaagggaagaaaaagccactaaaagagaaatttgacatgtaaaactcatgaataaataggcaaaagatgatgattcaaactaatcattgctattccaaaaaggcagaaaatacaggtgagactctattctccttgaaagtcaagattagacagcagatgttggggaacaggtgttcctactctggatcttcctgccgtcattgttgggatgacacattctcaaccaaggttttttctatctttgatgcattagtaacagcctggaagtgctagctacttctgtatcttctggagatctaaaaaagtcaaagctgtgataaaaatgcagttagagaaactgagcacctttggtgggaactgcctattaaatgacacatgtttgtagcagagtcagattactaaaattactcagggtcatagggcaaaatttctcccatatattttattgattttgttgagagttaacccatggctctggtcctgaacaaaatgaagactctcgattttggtaaccctagacagaaatatttgcaatgaataaagaaaacaggtctgatccagagaggcaaaaattctgggtttgcacaagctcatttaagcctctgctgtgatatttgagcatccaaggtagttttttttttttcacctaccaagtaacctgtaagtaaaacaaaacttataaaacttgttccctgttagctaacaatgtaagtttggagaataaagcctgaaaagccgtttgagtctcttggaaggaaagccccatgtaagtacaaagtatcgtcgtgggctttgaacagctgttaagagattgtcagagatctgccagacagataaagttggctaagagagggtaccgtgtagacacattttgtctgcctgccacttgcaacaggacatttctctatttgagctatggacacactatttaatatcactaagcatctcttggccatgtggcactgttctctgacatttctgtttaatgtttcttagttttattctcaactgtttcagttcacctcaagcacagcagtcagaaattgaaccatgtcccagtccctcttgcatgctgaaccatctaactgtctgaacacctaagttagggttttggctgacatctcatccaggtttttcactattgttaggtgttggaattcttgacagtggacatgctagcaggagcatcagattaatcccgagtgacttgatctcggtaaggatgctatggaaaaatccattagctccatggcaaggagcccgtcgttcctgtctgtgcatgctggactcagtgggacacgtcttctaaagagatgctggttcaacaagagtgaaaaggaatttttcataatgtggattatgtgtttgggagtgggaacagtgcagtttgttgggaggattttgactgtcaagagagatggttacttagaaaccattcttcctgcccatttgctgctagggttgctgttagtgtttggggttgtgatgtaatcagtgccatgtcaacttgtgaaaaagaagtgttggttatgaggatgaactttccaattctaggtaacatgactagaaggaacttttcaggctgagctctctgaatcttcctttcctggattgcaatgtttttagagattgtcagaacaaatataactttacatctttgagcattatcactggttattgacataggacatgtttctactatgcatagtaaaccaagatcaattatttccttctttctttctttttttttttttttcttgagatggagtcttactctgttgcccaggctggagtgcagtggcgtaatctcggctgactgcaacctctgccccctgggttcaagcgattctcctgcctcagcttcttgagtagctgggactacaggcatgtgccaccatgcctggctgatttttgtatttttagtagagacagggttttaccatgttggccaggatggtctcaatctcctgacctcgtcgtgatccacctgcctcggcctcccaaagtgttgggattacaggcgtgagccaccatgcccggcccaattgtttccttttagaggttcagttttctttgtctatgctatctttatttggaataattgtgtgtacgtgtcttttaccttgaccccattcttatttggggttcagatgaatgaaaataaaatttggggtttatttaaacaaattgcaaaactcaggcattactctcaaacctttaaatataacttggtagtgttatttgattgagttctttaattttagttcttgggccagggatcctgttatttttggcatagttttatttgctgaatgtttacagcatgtcattgttctttgtgttcagagatgttcatgacatcattatttattttgggtgtaactaattgaaagattggccgaaatacagatgactttctcttcagtttttatgaatagaagatgaactctggtttgttttacagctgttggtcttatatgttgactttgtggttgcaagagtattcagaggaatgtgttactgattgtttattaagtgttactatgtgattctttagatatgggtttaaaaaaccgcatctatctcattgtttatacatttactcatgtattcattcattcaaaatacacttattgagcgctgttgattgccaagtactcattagtcattggtgatataccaggggacaagaccgtctttcctttattcgatagttttctacatttctggttcaagttgctataacatacattattttgctaatttgaagaaaggtgataatatctttttttgttactgatctgtaatagtaggttctattggtaattgaagcagggtataggtgacatacaactaaaaatgtttaatggctgtaaaataattgtaggcccataaaaagaacattttctggaagaaaaagattatgagagaataaggtaggctaaaagagaaacctcattctaataaggctgacttccttctatcttttccacacagttaccaaaactttctttttctatgactgtcttctcaggctagctctatgatttggtaaatattaaaaggacaaatagtaaatcttataagttgcttagtgctgaagggtgaagctagtagtacaaggagaaagctttcctcttgtatcccacatgtaaatcagggacctttcttactttcccataatataaattgtatactattcaataagaagcttctagagagagtactcctcggttttattttctgactcagaagaaggcatctcatgttggacatttcagggcagagagaaggagaaggaaatctctaccaaagtgagtgtgtgtgggagagtttatgagacatgttgaccctagcagtgtcctccaagtcacattataaaggtagcagccttcttgttgggctaggcttgcttacctgcaaccctctctttgggatagataataggatggagtaaagtgacttttgaggttcctttcatttttttataattctatactttaaagagaagagattggctgaagatgttgaggaccaagttgtctttggtggatgctagtggtcagagaacagtgatcctaggagatcaggattatatagtcacaattgttttatcagtgcaaacctcttaggtttgtggggagcaaaggacactaaaattaattttataatgcttcaaaaataaataaatggggtcagacacagtggctcacagctgtaatccagtactttgggaggctgaggtgggagggtcaattgaacccaggaggttgaggctgtagtgagccatgtgcacattactgcactccagcctgggtgacacagcaagactacatccaaaaaaaaatatagtgctctttttggacagttcctgtttataggaggaaggtccacaaagtttttaagtagggtttttttttggggggggcgggagttggttttatgaataatagaaatttatttcctcatcgttctggaggctgaggggtccaagaccaaggtaccagcatctggcaagggtcttcttgctgcgtcctcatgtggtggaagagcagaagagcatgagcccactcccatagccatttttataagggcaccaatctattaatttccaaacatgaattttggaggatgcatgcagaccatagcagggaggatccttcctgcctcttccagcttctagtggctcaagtattcttcagcttgtggctttatcattccagtgtctgcctccctctccacatgactttctctttgtctctgtgtctgtatctcaagtctttctctgccttattctatttttttttttttcgagatggagttttggtcttgttgcccaggctggagtgcaatggtgcgatctcggctcactgcaacctccacctcccgggttcaagcaattctcatgcgtcagcctcccgtgtagctgggattacaggcatgcatcaccacgtccagctaattttgtatttttagtacagagtaggtttctccacattggtcaggctggtcttgagctcctgacctcaggtgatctgcctgccttggcctcccaaagtgctgggattacaggtgtgagccaccgtgcctgacctctgccttactcttttttttttttttttttttttgagacggagtctcgctctgtcgcccaggccggactgcggactgcagtggcgcaatctcggctcactgcaagctccgcttcccgggttcacgccattctcctgcctcagcctcccgagtagctgggactacaggcgcccgccaccgcgcccggctaattttttgtatttttagtagagacggggtttcaccttgttagccaggatggtctcgatctcctgacctcatgatccacccgcctcggcctcccaaagtgctgggattacaggcgtgagccaccgcgcccggccgccttactcttataaggatttttgtcgttgtacttaaggtccaccaagataatccaagactatttcatctcaagatctttaacttacttgtacctgcaaagattcttttaccaaacgaggtcccatttgcaggttctaggggcccagatgtgggtttatctctttgggaccaccgttccaccctctgctgtctccaaaacacatgtcaagtctgtccgcttttccactatcactgcaccaccatggttcttctctcctgggactcctgccatcagcctctcttgctttcctttctctaccccataactcatttccacacagcctgagggatattttaaaaccctgccccaaaccatgtatccctgcctgaggccttccagtgccttctcttacctgtgcagagctggtctcctgctgagaatttacatcttcacacaaaacgttatcaccttgaggtgagctcccagtgattttcagctcatgtgctcatgccactcttccactacccagcccagcaggccaccaagtttcattttattctcagcatttatcacaacttgaaagcattctctctctctctctctccttcattcacccatccctccttcaacccacctacctacccatccatttgtgaaattaaagaacagacttcttctttgtgaggaatatctttaacgtattttaaaacagtttcatcacctgccctcctctccacaccctctcccccgttttctcttctatcaatggtgggttcttcatgtccatggtgtctgggcttttcacaatcctcactgctgttttctatcatgctaccgggcagggcaggcaaaacaaattgagatttttttctggttaaaatctccaaacaaagttgtgaaaaaaaaaaacccaagtaattttagaaatgtcatttttacctaagaattgacttgcttgagtttctctttatggattgactattacctcattgaaggacaaaaggttaagatttttggcagtgaacttctttagatgtcagaccattgcttgggctatgcagcagattcatttccttttcagtccccacaacttgctgctgttgctttcctgctcatcacttctgtgaaaatcattgtccttttcctttttctcttttttggtctttgtttgaactgtcttctgctttcatttcaccattctgatggaaatctaagtgttgttttgtggaacaagtatcccctttgatgtctctaggcacttccatgcccaatttgtgacagaattctctgttttcatttctgcttcttggagacctgataccaatttgtaaacagtctcatgtatggtgctctcatgaccgatgtactgcagtgagtgagtgtgacgcatcacagtcctacatgcagggcaggtgttgttctcttacaaatactttaccaggcaacccctacagaatgtgtgcagacacttggtcaccctggttgcatcaatgaggtgcccactacacaggcggcaggtgatgtgggccttagtgtcccacagcttaatccttctggtcaacatctttggcttttcctttggatgtctgtctgcttgtttttctttctgccttagcatggaggccccacgtcatcactctgactcactgattctgcctcactctgcactctggttcacttctggcccttgctgtttttcagttttctagcacagaaacagttacttttgagggatggccaggagtccctgctacattgtgaagctgtgatcttggtcttgggggtacaggcaggggcactccaggagaaggaaggggtgccactcctttcggatccacccccaagaggaggagaaaacacctgtcaagaatctaaaataaagacatggaagtcctcaggtagttttaatggttcctgatagttttcctaataacctaaatttccatcatgtttcctctatccagcctctttttcaacttctctttaattagagtccctggtccatctttataattactccttcacacacaatcagtaccctttcttaccctctctctatccctcacactctcaattgagctcttctggtaaaacttgctgctggttaaatctctgtgcctactcacctctccacccatgcctgcgcccaagcagctgaatgtggcttgagaaaaactcacaaccatcttaacaggtctcactttagatatatgatgtcacatctgaagtgggcctagagggctgcctggcagtcctgccccatttctctgagaatgtactcgcctactctccaagacgactatttcacgctttttcctctcacctcaaaccaccaacatttctcttttcctttccagtttttttatttgaccgagaataaaagaaatataaagatcccctacccaccaaatctgcccaccttcctgcaccatgcctgtttctctgccttccctttttcctaccgtttcaggggaggatgtatccaaaccagcctgtgcactggaccttgtcttcttttagtcattatgaagaagccaacctgtccagtatgcaccaggcttagttttaagtaccttataaattaactaatgaaatccttacaacagccttgtgaggtaggaataatatcagttttacagacgaggaggttgaggcacagcaaaattcagcaacgtgtgcaaaaatcacacagccgtaataaatttgtgtggcttcagagtctatgcatgcagccactgtataataccatgtctcccaggatattctcccgcagctgcttctgtctttcctgcctcattgattgcccctttcgatgagatctgacccattaacttacaagcaggttctattttctatattctattttaagaccctacccttgaccctgtgtctctctccacctgccccctattttctctgttctccttttagccaaatttctggtaagaattttttgtttgtttgttgtgtcttcttttctaatctttttttgaaccactccaacctggcctttgtctatatcattcctccaaaacaacttttgtcaaggtcccccgtgccctcccaatgtccaagccagagggacaccctcagtctcttactcgggcccttggagacgctttcttcatttgccttccctgacaccacacacttcttggattttctcccaccctggtggttactcctgtttcctgtgtggatcttcctctgctggtcaccctctaagtggtggctttgcccgggaatgtggtctggaccatttccctttctgtctatattcaccttcttgataatctcagccagtctcaaggctttgaatattattaataaattgacaattcccaaaatttatctctagctttgaaacttccctagacttccaacattcccttggatgccataggccatcacacagttactgtatctcaaatgcaaattcttgttttgactccttatcttttagatcccgttttcagtcatgtgggatcttcctcctgcatctcacattgtacttatcagcagggttctaccttgaaattacatccccgatatgactacctctctcctctttccagattgtaccctagtccaaagcaccataatctattcctggtttttaagtggactccttgtcagccttcttttttcatctcttgctttgatatagcctgttctccaccctgctgctagagtcattctttaaaaatataagtcagtactacctgttccccaaaaacctattgaaataaataaaagacttaaaaaaaaaaaaagtcagatctggcccctgcctacttcttcctacctcatttcctgtatttgccctgtgcctcccagcccaggcttctttgccattgtttgaacacagcgaggccacacctgcctcaaggattaggtccttgccatttcctccactgggaatgctcttcctgtagatgttgacttaaattaaacaaaagaatgcattattttggtgtggcctctgttcaaactttaccttgtcagaaaagctttctttaatcaccctttctaaaatagcagtgcctggcgttttctatcttcttgccagttttaatgagagtgtagagtcacctgcctattggtgtatctgtttgtttgctgtgagtctccctgggtggcacatgggctccacgaaggcagagacgttattgcttttatccctagtgtctgcaacggcacccgttagggggttgatacatatttgttgaatttttagaatagtgacagacattaatcctacaaagttaaaggacatataagaataaaaaaagattaaaggtgattttttttaaaagttaaaaagcctctgaatgatagaggaaaggtagtatgggtggagaaagttgtgtagaagcagaaaaggagagtctcacaattcttgggtggctccagttcttcttcttcccttcttttctccaccataggaggagaattacctgttgcagtaactgctgtgccgagagtcaaggggagagagcaaagggaaggccttctgtgaactttgtgggcacttaccaggggcttcagaggtgcatccatggtgaggatggggccttgaagtattactaggaaattaacattggtgaaacaaaatgaaattgggaatcatatgttccttaaacccactccacttggagcattgaaaatgacagcttgtggtgaagtgccatggctcacgcctataatactagcattgtgggaggccgagggaggaggatggcttgagtccaggagtctgagactgtggtgaaccatgatcacgccactgcactccagcctgggtgacagagcaagactccgtctcaaaaataaataaataaataaataaaaaataaaaaaaaaaaaatcaagaaaacgacgaattgtgaatgttaactcttacaatgaagataatggtaatttttaatgtgctgcctttctgaccactcctggtgaatatcccacttcccaattcctttagagttggattgagaatgttgatgcaattatttccagtttcttggattactcttccttagagagaacaagatccaagagtgaatttatgtgctcctcacttaccatttttgtgagttctactcactgatgtggatgaatagaaaaagctgtgccaaaataatgaattgtctcctttaacattatgagctataaagggatgggaacagcttcttttaagttctgttagtatttcacagtgtgctaactgataaacttttaatgatctccagaaaacagcaaggggatgtttactacccttgtaaatataaataactttaataacaaatcatattacttgaactttgactcccagtaaacataatacctgatacttatctgacataactatggttttgaaagtttaaactctaggacatatggtgggtgtataatcctgcaaagttcacattgtctgactttggctataaagatgtgtttcaaaaggccaaacaattattttgtagtgtttggatcatttccaaggattcatttgtttcagcataaactcattatatgtttatgcttccctttgatttctttttgcctagagggacaagaagatgaggatgaagaggatgaagaagatgaagatgaagaagaggaagatgaggaagaagatgaggaagataaagatgcagactcgctggatgagggcttggatggtgacactgagctgccaggttttaccctccctggtatcacatcccaggaaccaggcttagaggagggaaacatggacctgttggagggagctacctaccaggtgccagatgccctcgagtgggaacagcagaatcaaggcctgggtaagagtcatgtgtatttcccttttcctcccttctcccctccttttatatttctcagatgacagccttctttcattgccatctattgtaaagaaaaatggattttctttgtttatgggtggtttctacatttatgggtggttaagaatataagtataaactcaccgaagaaataactgataattaaaaattaaaaatgatcacttatttcttcgatgagtttatatttatgcccttaagtagccaggctccaaacagtctccagctgcctattatgagtacagttgcctgatgtttggaagaaattcagtttttccttcccagttgggagacagttgtaggctttaggaggacatccgcacagggaggagatacgtgtaaccaggatgttggacaagcagcaccatgggacagagtcgaagaacaaatgcattttttgttccgctctgtcctttgggaatctattacctaggggaagttcttcccctgcttgtgggtgagcagttgggcagtcccatgatttttctgttcagacttgtggctgcattaccttgtcaaacacgatttcatttcagcagctagggtggccagaccactccatgctcactctctcttattcatggcccgtgcttgtggtgagagacttggaggatgcaggccgctcctcagctcagcacttccttccacatcgacagggaagctccccagtctgtccctctcctcttgtcaggggagacgctattaagctctcagtgactccttgaggccctattcccttgacctgaggtgaattaatctcttctccaaggaaactcttctctaaaatgctcctagtctttaacagctaggcccattgattccttcagtgtggcagatgttagggttaagagtcagaaaaactggtacaaatactagttttggggatatgtcattgaaaacttgtaagttggtctgggcctttgctctggatgtaattctgggagttgcactctatagattggcatacaggttgtagttgacatgtagctgggatttcctgtgtaatatgctataggaaggagagaagatagtaagggatgggcttccagtggacagacaagatttcttgaattactttctggccaaattcagcatgcattgggcaatgattactttaaaaacatgtcacatgaaactggtaaaaacttataagggtatcaaaacattataatatcaaaatactataaaacaaacacaaaataagattatctctggtctgtgaacaagtgaatgaatatttagtctgaaatgcttctgttctacatgaacaggtttgaaatgaatgaaataagatacaaaatacaaagataatttataccgaacaggttaaaaacagacacaaatctacacgtaaagtgactcctcgggttgtgcacagctttatactatggaggtatcatgtacagttaaacagcctttcagtatggatgggatgatattcggatgctctagtactttaccggagagaggagcgagggagacagggttgccttagcacagaattaggaacatcatatggcatctaccctggattgtgatcaaagaaatgaagtctcaccacttatgaagtggacttaccatctttgagtaattataagtaattagagggtcttagaaaactataaggaggctctgaatcaaggctaaaatgatcatttgggctgtggtattatcatgctcctcaatgcaaatttaccactgaataggagtcaagtttttaaggaatcagctttactgaggtatgtattatacacaattgcacccactgaaagcttatgatttgatgagttttgagagttgtatacacccatgcaacccccatcacaatcatgccgcagagcatttccatcatcccaccaccagtgattcctcatgcccctttgcagtgcatccctcctccagtcctcaaccccagataaccactggcctttggtcactatagattagtttgcattttctagaattttgtacaaatgacatcatatggtatataccattttgtgtctggcttagatcacttagcataaagattttgagattcaactatgttgtatgtatcagtagtttgttctttgttatatataacatacacacatgtacacacatgcacacactcacatacacatccacacacatatatatagctgactagtattcaattgtatggatataccatgttttgctcatctagtcacctgttgatgaacaactgagttgttccctgttttggctaatagcagttaaattttaaggcttgtcaggcttgaggcttaggacaaaaagctctgttgtcctcctgttctgactcctgatttcagtccccttgcacagctaaagttctttttttttttttttgagaaagcgtctcactctgttgctcaggctggagtgtagtggggcgatctcgactcactgcaaattctgcctcccgggttcaaacaattctcctgcctcagcctcctgagcagctgggactacaggtacccactcccatgcccagctaatttttgtatttttagtagagacagggtttcaccatattggctaggctggtctcaaactcctgaccttaggtgatctgcccgccgtggcctcccaaagtgctggggttacaggcatgacccaccgtgcccagccacagcttaagtttcttaaattagtgataccagtttagtaattttttattcttttaaaaatgacttctccagatcctgagttttaaaaatatatcagactttttcttattatttactatttctctaggtgtctcttactttctgctgttgcttctttttctaataaattcattttgtcaacatgttttcagcattgtgggtttcctgggaaccaaagcaaggaacaaatatttcaaactgttcttctcactgagaagattgtgtttgaggagtatctgggtctagaattttattagcccaccaaccttttctcggaaggacaggaatatctcctagtaattcaaaaccaataacaacttctggatcaatattaaatggttcagtttactaaagactctgaggtcaggtaaaatagtagaaactctttccttctttgtgatggattttgtgataatggctccccgaagatgtctatgtcctaacatctggaacctgtgaataaattaccctacatggaaaaagagactttactgatttgattaaattaaggatctttaggctgggcacagtggctcacacctgtaatcccagtgctttaagaggctgaggcaggaggattgtttgaggccaggagtttgaggacagcctggggaacatagcaagactccatctctacaaaaactgcagaaagttagctgagggtggtatgtgcctgtagtcccagctactcaggaggctgaggtgggaggatcacttaggccaggtgtattattagtttgttgtcatgctgctatgaagagatacccaagactgggtaatttataaaggaaagaggattaattgactcacagttctgcagggctggggaggcctcaggaaatttacaatcatggcagaaggggaagcaaacatgtctttcctcacctggtggtagcagggaggaaaattgcagtggggtaaaagccccctataaaatcatcaaatgttgtgagaactccctcattactatgagaacagcatggggaaactgcccccataattgaagcacctcccacgaagtccgtcccagcacacatggggattatgggaactacaattcaagatgagatttgggttggggcacacccaaaccatatcaccaggagtttaaggctggaataagccatgattatgccactgtattccagcctgggtgacagagtaagaccctgtctaaaaaaaaaaaaacaaaaagggaaggatctttaaattggaatgggtaggttatcctggattattgggtgggctcattataattacaaggttcttatgagagggaagtaggaggttcagagttagagaagaagatgtgatgatgggagcgaggtcggagtgatgtagccatcagccaaggaatgctgtcagcctctagaagctggaaaaggcaaggaaactgattctcctccaacgcctctggaaggaatgcaaccctgccaataacttggttttagctcagtgagacccatttcagacttttgacctccaggactgcaaggtaataaatttgtgttatctgaagtttgtggtaatttgttatagcagccatagggaatggattcagatttcaataggtatttaaggagcagcagcgttttgcagcattttgaaagcagagcctcatggcagctatgctcagagcgtaaacattgtaaaattacgtatactttttcctgtaatgaatcaataaaaatgcaaacactgagatcctttgaaaagtaatcctatttaatatgataacactttaattggctgtccgcttcattcctatttttaaatggggttgttttatttataggttatcacctccatttttacaatatgaatggtattccaaagcaaatgctaaaacccatataaatttttaaaaatgaacagattgaggtgggtagtgaactaaaaataatgcaaactcttggaatagattatagtctcatttcctgtgaagtgccaagagcaggaacatagatgactttctaaggcaaagcagtagattcaggcctcaaattctctgtacttctgttttctgcactgttacatatgcattgcctctgaagctttggagggagagaacttagtttcttcatcttgctgcagggaattgtgactgtggagtgaactgtgtgttgctgggatagagagcatcaggagtgaaagttccatgttttgattgaggtgtattgactgatgggtcaaacgctagtagcatataggaagctctctattcacatatgcagaattaatttcttctttcatctaacaggttcattttcagatttattaacaagaacctgtacagagagcagacactgtgacccacaccacttcttatcctgttgctataataaatcacacaattatcatctttaaagtgtaaaaatcagtatgaccaaagatgaatcaacatatccattttaatgtttatttccaatataattaatacctggtgattcatttctttattcttcctttatttgttagaatgccttcagttatagattatagaaatcctaacttaaattggtttatagaatgaagaaatacatttttttcccctcaaaacaagaagtccagaaataaagctcctgggtctattgatgcagaaccccagttgtcaggagtgacacaagtttttcccattttgctctactactatccttggtgtgttgacttggccctcaggctggcttccttttagttgcaatggctggaacaatttcaggtttcacagaaaagacattgttcagagaaggaaggcaactgtcttttcactgtgcttctttctttccttttttttttgtgagatggagtcttactttgttgcccaggctggagtgcagtggcgtgatcttggctcactacaacctccacctcctgagttcaagtgattctcccgcctcagcctccccagtagctgggattacaggcatgcaccatgcctgtaatttttgtatttttagtagagatgaggtttcgccatgttgcccaggctgatctcgaactcctgacctcaagtgatctgcctgcactcggtctcccaaagtgctgggattacaggcatgagctactgtgcctggcccactgtgcttctttcttttttttcttttcttttctttcttttttttttttttttgagacagggtctcactctgtctcccagggtggagtgcagtggtgtgatctgagctcattgcaacctccacctctcgggttcaagcgattctcctgcttcagcagcccgagtagctgggattacaggcacacaccaccacagcccagctaatttttgtatttttagtagagatggggtttcaccatattgactaggctggtcttgcactcctgacctcaagtgatccgtctgccttggcctcctaaagtactgagattacaggggtgagccactgtgcctggccactgtacttctttcttaagagagaggcagcctttcgagaatcccccagtagactatatcatgcccctgcttggctgagattaggtcacatgcccatttctgaacaattgttggcaaggagaatgtgatcacatgatgggtggaatggatggagccagagggcagttaccacgtgtgtcatgcatggtttctgtgttgttctattttcttctcttgttggtgatacacatctatgtctcttctatcctctaagaatcccatgatcaatcttgttaaattattatctttatttggtttgagttcatgagttctggatgagagcttgagggtggcattgttccatctcaagaagagaccatttttgtctactttagaatttgattgatgttccaggtggataaatctattggtttgttaattgagaattattatagctgtctttattttttcaaaagtctacatctaacattggaatggacacttgattgaggaagtggtatatttttctctagaaatccttaacaaacatgatgctcaattaatggtctagggatgtttgtgtaaaacagaggaatggaggggagcactcatgtatctatcttttaaccagatattcattgagcacctacaaagtgtgtgctgtatgttctgaaaatagtgagaggctgacagataaagagatacatagtcatggggcagagtgtggtctcataaatgtgaggtcagtggctgagcaattctatagtgagggactgaattatcacttatcaccagggcttgctttgagtgggcagccatctcatttgaggtgtgacttcaaggatgcgctgtctgatcctctaatttatatcagccttgctgtcctcacacagcgcctgctttccaatattagagccttcctttgcattgatacgtgtcttcagaaggccttagaaaaatgcagaactgctatctgggtagaacaactcattaaaacattatcatttattatgcactgtaagggattaatttatcaggttcattagctgctaatttggcaagatgaaagggagaggaacataagatgcatgtgttttgaattggtaggagggtataatgcattgcagcctaagtgtgggaagtaggccttctggaaacagagcacttgactgtgaggaagtgagacccttagccctaaaatatgtcagcactctcttctcttcctccttttttcttattatccatttaaaacacttaacatgtagctaagattgactacttaatatagtgcaacacagattgtggcagcaaggaaaagatgctttgcaaaaaatagtgggaagtgtgagatttcaaacagctcctcattgcaacaaatgtcagtgtattttgatgtataacatgttaagctatggcctaaaggtttgctcgaagctgccaagactaacataattaagtatgtgtatggaatgagtggaaggaaagaaacctcagcccaatgtaaccagcacaaagagagatggtagattggctaaagcccccagatgactgctctcaatcttggctgccagtggaatccatttcttagaactcctagagagctataaaaattattgatacctggatgctacccctataaaacaactgtcttaaaccgaatctactggttgtgccccaggggtgaggcagcttctgtttatgtagggagatggagaaagagcattttcagaagtgttgctgtacattgtgaatggaatccccatgagcaaaaatgtacattacttttaatgatctttcatgtgttggattaatgtgtcttaaaaaaaaccctcagctttttataatcatatagagatcaaacaaagtcaatattcatgatttaattttcccggaaagctcatggtcacattacagactcctgaatctggaccatggaggtgtatactcagacacctgtatccaggatgagtgggcttctctcagtaggagcttcttcaagtaggtatctgccagacgcctgggagtccttccatccccaagatggtacccagggtttctgaattagagagtaatgccattcactctttctctggaaagaatggaattatttttcctagacaagagaagcaaagacttgggtgcagacctctttttttttttttcacattagctaagacaccctggtgctagagtttaaagcagagataatgatctctgccttgtgaggatgaacatgtaaaagaaactgaacataagagaaatgaacacataagaggatgccttttaagcctgataattttttttattcatagatctttccatttgagttgttggttacttgtggatttgtagtagttgggaatttctagggaagaatatctatatgcttgtggtaaaggattcaacaaactcaggattttagtaggttttaaatgcacgtattgcctgagttttcctcttatgtatttatatcctgctgtatcgtacctatttgtgctagaaactccaaatcctctttttagcatgacgtaggttataaataggttaaatacccaaaaatgaatagacttccatcctgctgctcattcccaactgttctcgcagatgtgcactgtgaactacaaacacagaattcattagaaccaaaagtcagaaagagaactggccaagcctaaagaaaagcaggtgctagcctaaaaagaatttaaggtactctgtgtttttcttttatttttatgttcacgagacttctgatgaacagttctagatattggttacagtcaaattcttgttttcctttaacatgagacactttaaggtatttggaaagaggaaaggattgatggcatagataattttgtgggttgtccaagtttttcttgaagactgacatttaatgatgttttcacagctatgtaggaatttcacaccaaaaatctcaggcagacagcactgaagcttcaggaattcattaattccaagagtcatagacagaacccagatgaacccagatgatgttcactttgcccaggtcagctgtggaacactgcaaattcccttctcctccctttaaattttcttttgagaaaatgaacgtgaagcagctctaagtctcaaatgctaatgtaatgaaggaaacccataaaatagagttagttgagtgtcgctatagtccagtggccaccatgagaatttagtatgtccagagcactcagctaacccctcaggtgaaaagcctccctgaggatgttcctatgttgaggaagaaaaattaaccttggttgttaaaataacaacctttggaccagctgcatctcccgactcagggttcacagaagcaatactataagtagtaggttacgtaggatctcctaaatgaactgtcaagctggcctcaggaagctgaagttatttttgaaattcaaaagaattgatggtgcaaagtgaaaatacaacaaaccaaaagcgaagaagaaattaaatgttaaaacaaggaatcagagatttaaatcaggaaattagaggagccaacagttcaaggttcagaagcatttcttaaaatatctttatctcttttatatactcttggtatatttagggatctctgtgtgctgtctggtctatcttcctgcctgtttctttttgttgctctgtctgagacctctatcaccagaacatgtatcaaacattccctaccgactttttctaccattgtttttctttcttggaatagttgtctcccctttccatgtgtctacacctggatctggccgaagcctttcctgggaaccctaactcacgctgagcccttccttttgaaattcttccagtcatttgtcagtattatttaatttctattcactgcactatctcattttgcttatttttttggggaacctcagaacatctagcacacagcatggagtactgttgatttttaaaagggatttttcttccagctgatcgccattctgggcctgaaattttcaagcacagttgggaggagaaaggggatgctctattgcatctctcctggatagtaatgtggggggtggggcaaggtttttagataacataaggttaatttgtggaaaacatggtaactgatcaggtactgagggttttctgtcatgcttataactgacactaagcttagctatcatatggcaggtagagcttcacctcattgttatttttgctggttctacttactccaaagtgtttttaatatctacagagtctttagtaatccactaactatagcctgctttctggtcataaactttagagagtattttgaggctagttttttccagaacatcacctaacattttattttttatttttggaatgaactttgagtgattcttcactgtagaaacgatcagaccttagaagcgttgagtcaattacattgatcagcttagccgctgaatcagaggaaaatcagatggcactctcatggagtggttgagcaatttaaaaatttacaaatagttaattaggtggctttatagacataaagcctttgacagctcctaccaaagaatgaaaacaactattgcatcacctagatggaagtgagacatttataaacctgattctctctgccactggttcattgcttccccagttctgagaatcaagtcttctttttgtatctcacttctgaatgtagaaaattattattacgatgtagcattttaaatgttttaggttcctggtttgaaaatattttctttttgtttctttaatgccctgtaggagctgttgggttaatggtaccaaagtttcactaaaagtaaatttgaacttgttggttaggaagagccgtctggaaacctaaacccaaatatcttggctccaaattatctatcttgattttatttcttcttacgatctgtgtgaaataatagactcaatgcttcttagaagctgaagctcttctctgtattttcataattaaattatattgaattagagaattggaaaaaatcacatttcttttattacaaatgcatttcttatgggaaaattcagcaatcacttttcctttggagagccagaaaaggagacttggaactaatccagaaaataacctcttcatccactacagttaataaaaacattaataagacatattacatctgaggagaatgctgtgaactaatttctttgtaatcagaatgcactcgtaggctcagttggggaattattgaaatatgaaaacatctagtaggctcttctgggaattaaacaagaagaaacactaggaaaactgggaagtttttcagaaaggaagaaattaagtataaattgtcctctttgaaaaaatacagaggaaagtgaacaaagatgatgttaaagagtttccaactgttaaaacaaaatggagaaagtagtcaaaatgggtccttatctcttgatttgtggttacatggtgctaattaaggtgaattgcttaattaaatgctcttggtccactctattaagaatctggaatatttggagtctcccaaattttacttgttttacttaatctggatcagagaaagaatgcacttaacttttgcaattcagaaacagagcaattaagtaatttttccctgtctccaactgagaccataaaaaaggatgctaaagccttaaagcgaagcttagtgatcaggtgaagttgaaatcaacttgtttatttcatttcagcatttgagaattaatggccacagggttctgcttttccaactcagaacctttctgcttgcacagaatgatttacagaatctggcacaaaatatagctatatggtcaacaacaggtcccagttcaattcatgtgaagaactttcaaaataaactttacaaacagattaaagacccagggaaggaatttaaaatgaagtgctcacaacttaggggatgttgatttacaccttccatctctcagttttcctgcagggctgaaatctttctgaaataatgggatttgaatttttgagctgcttctcttgaaagaggatagtgactgtttggaacaggaaggatttgatgaatccttgggggtaaatacctgaagtcatattggcaactttcaactgccgaagatttgtatgaaatcagccataggcattgggtgaactaccacctgggtttgtttatcacaaacctgataaagtattgcacatcccacaggaccagagccttgggcttctgagtcctgccacgggggaaatagaccaatagtagaggccatgcacacagagttaaggtgaagagtggtttccaactagacctaacagctcaggatccagcaatggaaaccacagagagatagaaagacaactcatttatttactccagtagtaacaagaaggtggggcaaaccatggggaaaattctgtgctagaagtttgtttccagtggaaccttgcagccttttcttcttccttcttcctggtcatcattctcactttgcattcttcatttgagcacctgccacacctccccaggactgtcctgggatcccaagaggcttcactcttctcttaatccccctcactgtatcaaaacactcttactttaaaaccttggtctagtgactttatgatgggccaccaataattcatacttgttaaaagataatttttaaaataaggtataatcatgattctcctatagatttaaagtgaacatgtcagagattttatttaactcgttattaatgaagaagccaggcaaataccataactggctcaaatgagacatgaaagaaccaccaatctatgtggagtcaaaaattgttgaaatgaatttacagatggatgcaagactgactttttccagtcagcaagggttgtcccttttctggatatttaatttgcttgtttagagaattatttgttttgctatttgttatctgtaatttaaagagttgtaaaatagttcaaaggcagtgacaggtgcacagtcctaatggagtcagataattcttaggggtgccagcactccattgaaaggagtttgtttgtttgtttgcctttgagatggagtcttgctctgtcgcccaggctggagtgcagctgtgcaatctcagctcactgcaacctctgcctcccgagttcaagcaattctcctgcctcagcctcctgagtagctgggactacaggcacacgccaccatgtctggctaatatttttaattttttaagtaaagatggggttttgctatattggccaagctggtctcaaactcctggcctcaagtgatctacctgccttagcctcccaaagtgctgggattacaggcatgagctaccatgcctggctgatacttaatatctttccattattccaaaggttaaattttgaaaacatttttatagtgaaagaatagcttgctaatgaaaaaagttgacttatggactttggataggtgggaatgtatctgtctgcaaatgccagttgctataacaaacaactctaaagtctcagtggattaatatattggttccttgccctcttcataatccactgtcactcagttattctttcacccatatctccatcatcttcttggacctatgcaacttcactggatcctctgcagctagccaggaggcaggaaagagagagaacttagggaaggctacctgggactcatcagcctagaatggctttatttttcttccatttcaaattatgttagtgagaactagtcacatgaccccagctggatggaggaggggcagggggtgatggcagtacatatcatttacctgaagcagcctggcttggtgaacacttagcattttcaggggtggactttctcaaggaaggcaagccccagaaaactgggaaaactccatttgctatttataaatttttgctagatcatgagtgattgagcagaacaatgtctgctctccaggagctgatgagtcatggggaagaaaaatgtgtaatgcgaggcaggatgtgaaatatgctggggaagaaaaatgctctgaaagtaccaagtggagccttgtaaagcattacagacaacatgcagggtggcagttgagaggctagaatttggagtcaggaagtcctgaactgaagccttcactttgctgccattgaccctggatgagtttttaacttctctgagcttcaatttccttagcttgaaaaagagcatactaattattcactcataggatagatctatgtgtgcaaagtgaagcaaaagcccatttatctttggccttgaaggttaggcagatttcaaaaggcagaaaggggcaaaggagcattgtaagagaggagcaggtgagccgctggagccttggttcctttacccactacttcctcgagcttttatatattgggagttcccaagatgactgtatttgaggaaaaggaatccactatcaaaaataaatttgaaaacccagagtgttcttgggagtattgctgattcattgctttttccaacatgacccagtggttgtcagcataatctttgactgactgttaagtttaaccagtttagtgtgaggagatttacagttattgcctgtcttagagatgacacaaataattgatgagagtttttcacttttcagatattactttttcaagttataggtaagctgtggtatcctgcctatattttagcatctaaggtttttttttttcccttcccaggttaattatatattttaatttttggaacatgtacgatcattatgggatgctattaagaaagaaaaaaatcccaacatgactaccttaaaggcatttagttataaaggctgtagcattaagatatgaacttggccaggcgtggtggctcacgcctgtaatcccagcactttgggagaccgaggcgggcggatcatgaggtcaggagattgagaccatcctggctaactaacacggtgaaaccccatcttcactaaaaaatacaaaaaaattagccaggcgtggtggtgggtgcctgtagtcccagctacttgggaggctgaggcaggagaatggtgtgaacctgggaggcggagcttgcagtgagccgagatagcaccactgcactccagcccaggtgacagagcgagattccgtctaaaaaaaaaaaaaaagatatgaacttaaaatttgagagtgttttgtactacttagtagcctcatacaagcttatatttacaagtttatattcaacttctctgcaccataattgttttactcattttgtacaagcgagcacaatcctttggagaaagcaagcacagtgttgaccattaatgtgcacacagatccccctggggatcctgatacaaggtgcgttctgattcagtaggtctgggtggggcctgagacgctacgtttgttcaaggctgccaggagagactgatgctgctggtttgtggatcacgctttgaggggcaaggggccagaggatgcactttttatctgagaagttgaggaaacctcacttgggagagtaagagagactctttatttatttatttatttatttatttatagacagagtcttgctctgtgtccccggctggagtacagtggcacaatcttggctcactgccacctctgcctctcaggttcaagtgattctcctgcctcagcctcccaaatagctgggattacaggtgtgcaccaccacacctggctaatttttctgtttttagtagaaatggggtttcaccatattgcccaggctggtcttaaactcctggcctcaagtgatctgcccgcctcggcctcccaaagtgctaggattacaggtgtgagccactatgcctggcctatttatttttatagaaacatatacatttctttattattttttattttattaatgatgaaaatcttaaaaaaaataaattcaacctcactgtcacacaatatagccatataacaaacctgcacatgtacacctgaaacttcaatggggcacaaatgatcccatcacccaggtggtgagtatagtacccaatgggtagtttttcagcccttgtctccctccctctctctcccttctagtagtccccagtgttagttgtttccatccagaggctctcttttaaaaaggagttagaattttataatatctacttctattagggctacattcgtttggtccttctcatccctcattacagacagaattctttagacccttagactagctagttttgaccgggcacagtggctcacacctgtaatcccagcagtttgggaggccgaggtgggtggatcacctgaagttgggagtttgagaccagcctgaccaacatggagaaaccccatctctactaaatatacaaaattagccaggtgtggtggcgcatgcctgtagtcccagctacttggtaggctgaggcaggagaatcacttgaacccaggaagtggaggttgcggtgagctgagattgcgccattgcactccagcctgggcaacaagagcgaaactccatctcaaaaaaaaaattttatatatatatatataaataaatataaatcttaatgatagtgacaaaggcacctcatctccttgtcttagcatcgttggcagtcaaacctggctgttgcctactgcagatgagcaatgagggtgaactatgtcactcttaagtgctgtgtcaaatcttactgacaactctttcaccataatccagttactcagatagatcatttctatagttactattttgtttataagttgtgtggaattttagaacttttctgtggctggttagagtagtgccaaggaagccaacccccagctcccctcaggtaaggatcaaggttcaattctagaaaagagccaggtgtatgtgcttttcatcacaagagagtcctagtcagagcagggttggaagggcacatccagtcttgctcacgggaggaaaatgttccaagtacctacttattcagaaagtttttattgaatacctacttttacatagagtttttgtgggcacataaatatatggtaaatttagcagtagtagctctcatttcctgggatctttctctatacctactatggtttgaatgtgtcccccgaaatttatgtgttggaagcttaatccccattgcaacagtgttgagaggtggagccttgaagaggtaattaaatcatgggggcagagccctgttgaagggattaatgcctttatcataggagtgggttagttatctgggggtgggctcctggtaaaaagcgggagtgcaacccccttttcttttcagtattgtatgtgctctcttgcccttctgccttctgccatcggatgatgcagcatgaaggctcttgccagatgcgagtaccatgcttgtgtacttcccagcttccagaactgtgagaaataagtttctttttttctttataaattacctggtctcagatattctaacagcagaaaacagactaagacaatgcccatactgctaagcacttgatatactttacatcatttaggttttataagtagttatcattattcctcatttacagttaaggaagcctaagggtgtgccacaggtcaaaagggtttccaaattgcatggtctcaaaatagctgcaacattcccaagcatcatattcagatgtgatgatgtgtggcagaaaagagggattattactttttatgagcctttcgaagaaagactttcccagaagctgctcacacatctgccttcatctcacattggccatggttgtatcatatgcctatgtttcaaccagtttctggcaagaaaatggaactacattggtcccattggcttaggccaggggtcagcaaagtacagcccctgagacaaatccagcctgatgcctgtttttgtaaagaaaggtttttgggaacacagccatgcctacttgtttacttatttctatggctgctttcacctcacaacattggaattgagttagttgcaagagaccttatgatccataaacccaaaatatttactatctcactctttacagaaaaagtttgctgacatctggcttaggccagtcaagatctatatctgtaccattgttggtacatggttggtacatgggcagaagtagggattttttcccaacaaggaaagagtgagtggctgttggataggcaaccagcagtgactgctacagaagggcctctttatcccaatgaagcagaggtgctataacagacttttgtgtggggaggtggctgcacctcagagacagtacatgattgtacaggttacagccagaggaatgcattggatgagagggtaaaaatagaaagaaaaagaagtgatgactcctaggtcttggcatgtgtgggatataaacattcctttcaaattcagattattaaactagtgcagggcccatgcagttcccagcagccacatgcagctgtgacagggagcaccagcagctgaccagaggagctcataagcttttgccctgccctgctggtagttttaatttctgaagatcaaggagacaagaactgctaatctaatcaaggaagaactggtagttgaaacatgagaggtaatgtctgtttcatcttagagtaggagagaatgctgggcttatttaggaatacctccggacttcagcaaggcaaatatcagaaagctaagggaaaagtatcatctctgtccattgagtctacatgggtacacagtacacaattggtagtagttcctgaaaatttctattctgatagtacagctacaaattaacctgatgaggaagcaaagggtggaattttaagtagcaatgtggttttaagaggaatttggatttcagaagaaagagacaggatgtgtgggagcagtggagtaaaaaagatgtggattccaatcttggttatatcacttgctagctctgtgaatttgggaacagagtttaacctctccgagcttcagttttcctatttgtaatttaggagaataatatttacccatacagagtgttcttgccagccagtgcatccactcttgctctctctctctcttttctttttgaatagagtctcagtctgtcacccaggctggagtgcagtggtgcaaacacatctcattacaaccttgacctcccaggctcaagcggtccccccacctcagcctcttgagtagctgggaccgcaggtgtgcaccacgcttcctgactaattaaaaaagtattttttgtagagatggggtctcaccatgttgcctaggctggtcttgaactcctgggctcaagccatcctcctgtctcagcctcccaaatggctgggattataggcctgaaccaccacgcacagtctactcttattctacctaattttttcactctgcctgatacatggtttcatcaattctcactctttaccatctttaaatcttactcattcttaaatagaacaaccacccttttgtaaccagtgtgttattaatcactaaacgtgggttagctcagggctgtctagcttgtttcttcagcctcccatggtcctgcagacccatcaaatacatcttaccacctagcatccatgaccccctcattgggcttatgactggtctgggacgttggctgtggtatggagggcccactggggtgttcagaaggggccaagctaagcaagattatggtataggcagacatcactgctgctggccgggtagagggtgaagccttggtgtagacaaagctaagtctagtggccccgatgggaggaatgaggggcccgagtctgggttcaactataggattcttggcagggtggctggtgaacagtcagttagcagttagttgctcccaaatagctcatgaacactcaagcagtgctcttgcaggttcccctttgtcaggtctgctgtcatcaggtggtggccctagtcccttctttctcagtttacccaggatcaagttcctaccacctttatccctcttccattttccaaagctttgttctcattttctttctttttaaaaaaacaactttactgagatataatttacatataataaaattcagtagtttaaaatgtccaattcaatgtgttcgagtatatttgcatattttccctaagaatatgggtgaggttttaagcaagtttgggatctgctactgcattcaggcagcagacctctgttttcttcttttgattctgaaaacagggttacaaattagagttccaggttgaaatgaaaatgcacttagtcctttccagtcactgagccacttacatagggaagaaaagattggttggattgctttaaataacagtagatagccatgattcttgggaaaagcaggataatgactttaagacctaaagggtcatttatctgctggcttcatccccacagtgttttccaggcacatgggcatcttttcccatttccacaaactttcacagaagagaatttgatcttctggcctggaaacttactctgcttaatgatgaagaaattcttcaacaacgacaaaaataatagttgccgccgctgcctcctcctcctcctccccttccccctcccccttcccctccccttcctcctctttctcctcctttcttcttcctccttcttctttcttcctctttcttcttccttctttcttattcttcttccttcttgtttttaaacatacatttcttcttgtgtttttaataagtaccaggtattggttataatggtaacttataagaccttatttaatcctcacaataaccctaagatatagatagtgaaactttctccattttacatatgaacaaattaaagctcaaggtaatgttttcagtaaatggcagagccaagatttgaacgcatgtttatttgattgtgaagatgctgctcttttttctttttgaccatcaccaaatttttattatacttttaagttctggggtacatgtgcagaacgtgcaagtttgttacataggtatacatgtgccatagtggtttgctgcacccatcaacccattatctacattaggtatttctcctaatgctatccctcccctagtccccaccccccaacaggccccggtgtgtgatgttctcctccctgtgtccgtgtgttctcatggttcaactcccacttatgagtgagaacatgtggtgtttggttttctgttcctgtgttagtttgctgagaatgatgctttctagcttcatccatgtccctgcaaaggacatgaactcatcctttttatggctgcatagtattccatggtgtatatgtgccatattttctttatccagtctatcattgatgggcatttgggtggttccaagtctttgctattgtgaacagtgccgcaataaacatatgtgtgcatgagtctttatggtagaatgatttataatcctttgggtatatacccagtagtgggattgctgggtcaaatggtatttctagttctagatccttgaggaatcaccacactgtcttccacaatggttgaactaatttacactcccacgaacagtgtaaaaacattcctatttctccacatcctctccaacatctgttgttttgtttcctgactttttaatgatcgccattctaactggcatgagatggtatctcaatgcagttttgatttgcatttctctagtgactagttatgatgagcttttttcatatgtttgttggctgcagaagatgcggctcttaatatataaagccacatccctctcataattttgtgagttccttccagcactgtgctggagccagcacatattgaattgcaagagctaatggttaaaacttttgggaatttttaaagtcagctcttaaacttagccattaatagaaatcaaattatataaatttacaactaaataaattatattacaatattttcatatgctcaaaactcttcacttcctaattattttactacatgttacttctgtctgggctttcgaggctatttacgtctattgcatggtatggtggaaaaccgcatcatggtgtgctactgtgtatcttttcccaactctgcattctgtgatgtcatattggtggttggaaattggccatggtgggaggatttacaccacagaaattaataaatactacaaatcagagcttgatttactgttttgttgattgtctagacttaaagtgatggagtaatgttaatactgcaaattaaactaaaaaaaaaaaaaaagaactgtgttgcatccactgccaaagcggagagaagcttcagtttaatgaatataattcatatcaggataaggaatagtttaacagtagattacatatctgatttaatgttcataaatttattgggaaaagagtgggttagggtacaactccacttgtcaaatggtagatgaattgcaagcataagttagttacagatagaaaaacttgacaaaaaccagtgaaatcatcttgtgagaatcaattggctgcatgggacttgcagcctggcatgaggttgtccccatgagtttatccctgcaattacttcatcttaagcaaaagcttgaatcaaggtgaatttcaggtttcctgcattcttaaattctgaatttttatggctgcttgttgtacttagccttttaaatgtgcttgttggtttatcaagagaaaggcaaatataataaaacctcaatgattcaattttgggatttgagatgatactttgttaaatttgtatggcctttttgtagtttactacatgttttagcacacatcttgtttgttcctctaataattctaacaaccctagcattgagataaacagggagttattagtgttaccatttactgttgaggacactgaggctcaggatagttcataacttgctccaagagcacagatagtgagcagaaaggccaggtctagggcttccgggtggtttccttgatttctcacgtctcttctctttgatctgcttaaacaaagaactccattgagctagtagtagtcacatcatcttatgtcttctgagcatacgttgaacaaaaaagtctgtctaaaagtagattgcaaggttagaagactatttttttttcccagtgaagtaaataaacccatcctttctgattaagtgaggagcttaagaggaagtaacagttgattgcttgttaggataataactactttagaactcaaagtcagtgaacatgaatgtgcccacaccagaataggtgttcagtaatagaacaacctgataaagaggacagcagcttgggatatgtttgagtgtaatcacaatcaaagcagcctctagtatcttttatactttttatagcataaccagcagattgactttagtgatgaggataacacagagtgacttcttaagtgaacaagggacactagaaaaacaacagttttttattttttaaaaatggcataagaatgattccttatactactctaggcttttggtgtgtttcttttcttcttctcatcatttatttagcttggagttgccatatttttatggggcaaattcccaaagaggtgggaatttcatgaagccaaatatagacataaaggtggtattcagataaagaggaactgtgtttgctatgcatttcctctagtgaggcaaatgtatgtgatatcgtcccttctgcttatttgagcaagcccccaaggaggggcgctgtagccagaagttgctatttagggctctgtccctaatttactgtgatatgttaatgaaatatacacagaagtattttctccatggaatggggatacctgtctgtaagaatcattatgcatgatgacagggtgacatccaaacaccctgtggtcttgtctcaggcaccacctaccatcagcaaattaaaagatggctcttccttgaactcagcagtaggtgtcgctgcagtcaactaaaaaggaacgctctggccaaaaggattcttttttttttttagatggagtctcactctgttgcgcaggctagaatgcagtggtgcgatctcggctcgctgcaacctccgcctcctgggttcaagcaattctcctgcctcaacctcctgagtagctgggattacaggtgcacaccaccacacccggctaattttttttgtgttgttagtagagatggggtttcacgacgttggccaggctagtcttgaactcctgacctcaggtgatccacccacctccgcctccaaaagtgctgggacgacaggcataagccaccgtgcccggcccaaaaggattctattagagggaaaaggaaaggagctattacaattgaaagtagctattacaattagttatctagatttctttcccaccccaaacccaaatccttcattacatactattgtttatcttttaaaatgtgacgtcttataatcaataagtctcttcagaaaaaaaagttgcttctgtaaaccttgcaatccatctatccattcattcattcaacgaatacttcctatgtgcctacaacttgtgagggacttgctaggctttaggcttacaatgataaacagtagtgatagtctggctttttggagcttactctctggtggggagacaggcaattaaagaggtaatacaaaaaattatagtacagcaagattagtataatcataggaaatgcactagatgaaatagaaggagacaacaagggaaactgagttgaggggtcagagatgatctcttgcataaaatagcatgtaaaaaggtgcccatggggcacaaattagaaagacagcatctggattcctttactccatccgacattcgcacaggttcttgtcatggtgcattttatctcccttgtcgtcagcactatttgaaatgcataaagaatatccttcctcctgattttacttagataatttgaaatacctaaagggttaatcatgatttatattgctgtcattatatctctcttgagatactgaaaactgcttagtgtcttgtcaaatactttgacaaatactcttgattattttattagcatatgatatatgaaagataaaatcaaatgttgaagcagtctgtctttcccttatttctacatttactcctcactggggtgtttgagttaaaaaaaaaagcttcagtttctatcaggaagtcaaagtcagcctaaaacacaagtgggagccaatttggcaccagttatatgacctgactggcaataaagaactggctattgcattaggtcttggacattttaagaagataatgaagaaggaaatattgataagaaacatgaagttattctatcggtatttgctctatctctagtgactcatgaattatattttcaaaaccctaaactcaatttattaagagttccaaatactgatttttggatgagtaaagccaggagaccaaaaaaattgtatgtattgaaaagatagcagaaatgtgttctgatgaatcattaacaacttttcttgcctcttttatcattgtcattgcaaaagttccttattataattattacccgtactattgtatataatatatataacgtatattatatatatatattttacttgactctggggcctgcactcaccttattcatccaaaagaacatttatttttcacattataaaaggtatggcctcaaggctagtaggactataaccatttttcttagaataaagaaaataagtgttcaaatgtatcaggttacaaaataacagaaagacaaacatcacatgttctcatttatttgtgggatctaaaaatcaaaataattgaactcatgcacatagagagtagaaggatcattaccagaggctgggaagggtagtggaggattcaagggagctggggatggttaatgggtacaaaaaaataaaaaaatagaatgaagaagacctactatttggtatcacaacagggtgactatagtcaataataatttaatcgtacattttaaaatagcttaaagagtgtaattggactgtttgtcactcaaaggataaaagcttgaggggatggataccccattcttctccataatgtgcttttgccaagagctggacattcagaacagtgaaaggcctaattgtacacagttcttcttagcttttgtagatgaaccccttgccatttttatgtccaggaaattgaagtgtttggtcttgtcctaacttgtaaagggaaacattgtaatattctgggaggagccatggggaaacttcttattgctctagccttgctattaatttatggtgtggtcttgggtgactcttagttgctctgagcctttactgttttcatctgtaaaaaggggcctgatgtgaggtcagcatgtagaaaccccttcatttttcctctgtgctcacccccagtgtatttgggcccaccttcctttttgactcaaagaaagaggtgacctaacccctattcaagtacagccctgccacctgcatgcgatctggagcttatcaccttcttcctcctctttgagctgttaaggaaactgagccatttgggaaccctcattgtctaagttagaaatctgggtgccatctctaattcttctctgcttcactcggttctcgcatccactcagtagcaaagccccattcattttgtctcctaagagtgactgttcaatttacccttctttgccatcctccttgccttcagccagcatcatgtcttgcccaaactattgcaggagcctctttactgatgttctttcttgcttgcagttcctctatttccagtcccttctccacactgtgttcagagatatctttctaaaacacaaatcttattatgttaattgagtatcagcaactccactgaacaaatatttatggtgtgaatgggtgttataacaggttctgagagatgatcaatttgaataagacagcatggtcccatttaaatgaaatccttgtgttggctacctatattacctataggataaaatgaatttcctttgcatggcctttgatgctgtacgtcaaatttcccttcctgtagccttacctttgaatgcctcttctcttgccgtctgcatactttatatccctgccaccaaactctttttaacctgagcatgcagtgttctttcatgtttctaagattttgcatatccagatctctatgtctggaattccctttccaacttctcccttggcctacccattgttccctatcagatgaaactgtcatcttatttgggagaattctcattaatctattacttccttgtttatacttgtacagtcctttattcatagtgttatattgtatttttaaatgtctgtcatatgagattgtgagctacttgaaggcagaaaatttatgccatattcatggttgtattgcctatgatctatcatagcatatagcacattttcagggctcaataagtgattgttgattactaaaataaatgaatgaacgtactctgcatacatgcatgggtggaagtggatatcacaatgtttcttatctcaatggaaatgtcagtaattgtggaaagaagaaacttggaagttaatactagctttgatcttgttgatcttccaggtaagtctttgatatcttcaggtagatcatgaactgatgatggtcaggggatgtttggactagatctttatagatctttagagtcctttttatctctgtctctcttttttttttttttttcgagacagagtctcactctgtcacctaggctggagtgcagtggtgcgatcttgctcactgcaacctccgtctcctgggttcaagtgattctcatgcctcagcttcccaagtagctgggattacaggtgcgcaccaccatgccaagctaatttttgtatttttagtagagatggggtttcaccatgttgcccagacttgtctctaattcctgacctcaggtgatctgcctgcctcggcctctcaaagtgttgggattacaggtgtgagccaccgcgcgtggcctagagtcttctttaagctgtaaaagttgatagctttattttggttatatcaattcagttccaagagttggaacactttcctggtaacacttttgtctgctggctgagaaacaagtgtagtttgctgaatgtatcttgtcactgtctcttttcctggtactctgaaatccacttctgtgttcctaaatgacagatcattaggcttatccccaaatgcattggttgtcaggtaggcatcattaggctagtgagatgtcttagtttcagctgaataaggcaaatatgatgctatctaatcatgacaagcacaagggatgtggcgttctgagaagcaaagaacatctttcaagactacaacattttcaagtgtaggcaattactctagtcattgaatagtgatcaatatataaagcttaaggtgggaatattaatgcttcctttagtgcagaaacatcagcatcagcaactaaagcttttttggaggggggttggggggtagacagaattaactgataactcctcccatcgtggaaagttgatttttgtagtcagtttgcagatatatcaattttcagggatgtgaataataaaaagaactaaaagcttttacaggatatcttttcagtatctgcttaactcacactcttgcttcctcttaatcccctagcacctatgtcacatccgttatcatcctgaaaagaagaatcattcaggtcacttaaatggtgcttcaatgacataccatccattagaattcacaaaaagccagcactttccaaagagaaatgtgacactctctttcacttgttctttctcttcttttccttacatgaactttcaacttctcttgggatttcttattttacatattttcataaggtgatcatttttgcagataatgacttatcacctcctaaaagcaagtccctggttggctaaaaggagtaaggcaggtgttagtattcatccatttcccccattttggtttatttgtttgtttattctggctatgaagctggatatactcaaagcaaaaacatttggataaactatataaccctggctttacacccaggaacaagtggataggtctctggctctgtccaccaccataaagaactcttttttggagcagaccgcacaaacacttggaagccacagaatgatgcaaatgtcttgctaccatctcccttccaggctatgctggctgtccttacacagattctcataaatccctctttcttggtctgcctccccacttctttcaccagtctctcccaggactccagcttcatcctgtagttttgtgaaccagccactaagtcctgttttgcattgcttagttaagaaggccactttcttcccactagattggaagctccaggagggcagacactttgtttattcacatgtgtctccttggttctagaacagtacctaccatgtaacagactctcaatacatttttgttgaataaataaatgaaactttcagacaaataaccctctctgtttctgtagtgtccacctctcatagtataatgctgatgccttaggctagaagatggaagggtacaaagcagataaaagatgaagaaaccccatcctgctgtatttcaaaagtgtttgaagaagatgggaaaactcaccaacaattcctctacgcataggaaacaaatggcctattctcttccagtcttttttctattcatttttccctagttgacattatattttttccattttgtatcaggtcactctaagttacattgtagattagtcaagcaaactggatctgctaaaattagtagtaaccatattttgtttatttgtgatagtattttcctttgagtctgatacaaatgattttcacacttagatgtttctaaaatcatgggtaagtcttgcggttagtgtatatacttaatgtcaacagtaaatcttataatccatgacctttcaagatccatgaaatatagtatttggaagtagtatgcagtacagctgtttttggtttttcctcatggaaaagcgaacatactgacgttccttctggctaaaaacgtttctccgaactcttcaaagccttcatggttcccccttctccctgtttatttttttataacattaattatttttattgaggtaaaatttacataacataaaattagccactaaccactttaaagtgtacaattcagtggtgtttaataaactcacagtgttttgcaatcattacctctatccatctccaagacaatttcatcaccccaaaagaaaaccctgtacctatgaagcaattactccccattctcctctgccccagccctggcaaccactagattgctttctgtctctacagatttacctattctggattccctgtttactcttcaaggtaactatgaagaacaggactggctttctcaactggcatgtggaggcagaggatttgtggcatggctgtgacacattataacatgtttatacatgctgagatagtattaccaggattgaccactaaacacacatgctgttggcagttgcagaattatcctggaagtttctaaaaattagtgcacattttaattgaagtaaatgaatgatcaagactactaacatggcaagaaaagcttctaatgatgaaataatgtgaagagcaaggcctagtttagctaggtcccccacgtcaggatctcagctttaaggaatcagtggaggctcctcctaagattcactgaagaggatctgcctccaagctcacatggttgttggcagaattcagtatcttgacagctgtaagactgagggattcaggttcttgttggctgttggctggaggctgcccccagtttcttgctatgtggaccccaccacaatgcagctcacaacagcttgcttcatcaaagccaatgacagagaaaatctgctggcaaggcagacattgcaatcttatgtaacagttatggaagtgacatcccatcatctttgtcgtattctattgattagaagcaagtcacaggccatgcccacacctaagaggaagagattactcacagacatagtagtaggaagtggtgataacatggagccacattgctgtgtaaatgcagtcatgcagtcactcatctcttcatggagctggttaatttctctcctccagtaaagaataaattagataatgaaacaggctcattcttaaggacagtgtaggcatgaagagaagctctgtcgagggtatgctgtatacttgcagtatgcatagaacctacaaagactcaccatttgtgttgatttgtcaagggaaactggaaatcagtgaggttcttcgaggaatatgataaataattatgatgcttggtaggtttagcctccatgtctgatttgaggtatctccctgagcctcaaggctccaaagaatgaaaggctgcaatcacatcataggcaacacatgcagatagattctctctctctctctctttttttaagatggagtctcactctgttgtcaggctggagtgcagtggcacgattttggctcactgcaacctctgcctcccagtttcaagcaattcttctgcctcagcctcccaagtagctgggattacaggtgtccaccaacacgcccggctaatttttgtatttttagtagagacggggtttcaccatgttggccaggatggtctcaatctcttggcctcgtgatctgcccgcctcggcctcccaaagtgctgggattacaggcataaaccaccacacctggccagattctcttaagtcaatggcatcgtttgacttgtagaattcatcctacaagatccagcattacctagatgtactctgaaaaaattctgaaaccatcagggggctctatttgatgcattcaagagtgaattgtactctggctgacaacatactttctgtgaaacatttttttgatcccaaatagtgttaaccctgcttgatcaacaatatcatttatctattttggctctgaattactcctcactctttcaaaaaatcaatggcacctagcacaatatcacatatatggcaagcaataaaaataatttggtaagttcttataaattcttatgaattctaatgcgtgcattccaaagaatgttttacattctttgtcacattgctctggtgctaattgtaggaatggagtttttcaaagtatcaacattattggaataagtgtagtctttgattgacttattggaatagaaaaacatccgtttgtataaatagttgtgtgtttaagagcattcttactccttgataatgtcgtcttctgcctcctctgagagttgccattttttgcctccaatattgtagtgaatacagtggttgtgaatttaaatagcaaagatagtgagaatcagaggtatctcagggcattagaaacactgacaactatcttctgcttattctttataggtgtttctaattagagttttgctaaattttctaattagagttttacttaggatcctatgttaagctattcagggagtgggtaataatgttttgagaaaagttaatgaatgcataaatattataagtgaccgtttcttgatatgataatcgtgtgttggaattgttaaggcaaaattttaatgccaacattttgggtgttggctcaacctattgttgatattgtaagtatagctaaatctttgttaaaagtgtgcgtattacctacaccagccatttacttccaggctggcttccttttgcaatctggctgtttccaagctacttcctttcactgctatttccatttgatataaacagattacaaactcattttttaattgcctaagaaaaatgtcaattaaaaaacagaccaaattccagccaggtggaacgtttctgtttgttagttttattgtttatgccactgcttgcctctgtcagtctgtgtgattgagagctacccgtgcagcttgacatgggcattgattttgctttcttattgatcaaggaagacatgagctgggaggttgtgatagatgacagtaccaaagtgttcctggcaaaagttgttcagccatgtccttctccaactcattaaatcaaagcagagttctaaaaatgatattttttgtagtttctgtagtgccttgcaaagctttcttgggatgcagataatcagattttctgcttatttggaaagcaccatgacctgcctactgatctttaataaaatgttattttaaaagaacacatgaatcttctttcccatctttatcttgaaaaactctcccccttgaacctatggcttttattatgggtcaaattgtgttcccccaacccagaagatacgttgaagtcctaaccctggggtcttgtgagtatgaccttagttggaaataaagtcttcatatatttaatcaagttaagatggggtcattaggatgggtcttaatctaacatggctggtgtccctataagaagagggacattttgacacagacacagacatgcagggagaacgccatgagaagacatggagacacattgacagagtgaggaggtcacttgaagactgaggtagagattggagttatgttccccgaagctaaggatcccctggagctgccagagccaggaagagacaaggaaggaccctcctctacctcctctagaggctctgtagggatcatggccctgccagtaccttgatttggggcttgtattcttcagaactgaggaataaatttccattgtttaagccacattgagggtggtgtgttgttacagcagtgccagggagctagtatacccccttgagtgactgctctttacttctcttctccccttcatggccaagctgcaaattggctagatcactccactctcctaaaattgcccttgctgaggtcttcagtaacctcccaggaggggctgcaaattatggagccttattgtctgcattcaagcccagactctgctccctgctagctgtatgcccttaggcaaattatttaacctctctgagacttagtttatgtttctcagtaaaatgggcctatctctgggggctgctgtcaggattaaaaaagttaatatagctagaacacttagcctcgtgcccggcacatagctaacatttagtggtcactgtttctgttaatatcatttgctgcatgtgtcatcccttttcagttcttccattgcctgactctgcagctctggactctgctggctgctttcttctccattccattgccgtctagttatcctccatgcctctgcctgtttcttctgttaagcctttcctgggaagacattgtagctaatgcatagagcccacgtgttggaatctaatagcactaggctcaattctcagctttgctcttctttatttgggggacaggaggcagagtcttactctgtcactcaggctggagtgcagtggcatgatcatggctcactgcagcctcaaactcctgtgcttgtgttcctcctacctcagcctcccaagtagctgggactacaggcatgtgccaccacatctggctaatttttatttttatctttctagagactgggtgccactatgttgccaggctggtctcaaactcctagcctcaagtggtcctccttccttggcctcccaaagtgctaggattacaggcatgagtcactgcatccagccacctttgctcttcttaagtgagtgattttgaagaagctagactttctgagcccctgtttttgtttgcttttttttttttaaagataaaatgggtaaatctgacctgattgggcttgtgtgaggagtaaatgagataacataagcagagggcccagtgcaagctggattctgactttaaccccacctctggctcatcccctggccacccccacctccaagttgaaggctgcctcgtaggctgtgtgcttgctgccttacacagctccaaggggtgacatttacttgaagtagaatgtgatgcagcagccctgcctcccttttccctgacacgacttttggtgcagagatggatctgtggaatattcataaggtgctgcaaaaaaccccacacgattgaaactatagtcatatttgacgttttatctcactagcctttacattcagtggttatcaggacccatggctttcatttctcctgtaactctcagaaatatcctttcttgaggaaagcgattaaccagattctggctttggagaggatagcagggaaagcagaaataatgtctgatggaaagctacagtgtcagtcctgatttcagaaaggattgattctaggcaggcagtgaggtgggctcggtaaggtggtgggaaggctggaagtggcccgtgtgtggctttacacggtaatggttatcactagtaggaggaggactagaggtataggaattggattctggtgggtggggtgtcaagatcacaccttggaggaaggactggccagtgaactggaggtcaggaaaggtcgctcgtcctagacttggcaattggttgaaattcaggagaatctgcatgctctggactgagccagcccagggctagggtgtcagggctgagagggatggattcgattcctccttaagtgtggcctggctgggctagtcaaggaggagcctcacggagttggagctcagcatggaatctggaatgtgatgttattctaaggctcaccaaattggtctgaggggctcaagcagggctattgatgcaggctgcttctgtcacccttaacttgggaactgaggtaggccaaggctgcatcagtggttccagctgagaggacctggaggggtgagagataaggagaataaagcattattgttgtgaccaccgcaagcattattgcttgtgattgcagaggcagtttgggaatctgggaactctaaaaacttcttccctttgtgcatttgctgtacatacaccgtgaaaagccctgtgccagcccaaacaccccggagataccttttctccctggttttcattgcttctgaagagtagtgacctttttcagacagagaggtattttgtaaatcatatgcttagaaatgctccatctggttgtcttatttttacaacttttaatacagattcgatgggcacagtttggggaggataatctacaaagtcaatgtcacagatgcttagtttttcttggttggcagtatctgggtctaagacttacaaatgccacagtgtttagtgatcaaaccacagtttcagagtaccatcagttaataaaatgtaaagttattttcaaagtctttatatccctaccagatcttaaccagtctttggttatggcagtcaatttatctgttagaattaggaccaaattgccttccacgagttagaataaagagggaaattagtgtaggggagtacgaagccaaggacatgggattcccttctttgtggcaaagagttatgaggaaataacagcaaagctgactcattaatgctgccttgtttaggagagtctagagatgaattaattactcagtcttttcagtgtaggtagacaaatgaaagagtgaataaagtggagctcaggaaaagtagaagtaaagacaattgtgatcatattaataagatccaaaatgaatccaatattgattaaaacggactataatacaatggacttaatatgaagacatgaaagcatttctcctttttgcaggttctagggtggttttgtgttttagtttgtctgaatctcactttattttctacgtattgacaacatggcttaaacctgtaaacatcctcctgacagatcctgttacttagcttttcagttgcttgaaatctggagctgtagtcacaagagagtctatcggtctgctcaaaaaaaacagggagctcacatataacgcagtgtctagggctttgtatgctgcacacatcgaaggatgatacctttggaaaaatgaacaagagtgtaaaatttttcctatgcattaataactactggctctgatcaaagtccacagacttagctggtcattaattctccataaatgccatacaaagagagaattactgcttcagagggttttcagtggcacatttttggtgaatgatgtggtataaactggtatttacaaggtacttttgagataattagtgttaagtattagttgaaccatttccaatagtccatttgaaaccctatccacaggcagcagctgaagggacattttgaatctatattctctagatcttgggaaggcatactggtcaaaaatgttgactttaattagagctcactctgatgaaatttagcatccttgccgggctcttctgaaatcttgccccgctttctacagtgatatcactgtctggggttgcacagagtgtttggcagagtggccttgaatctggcattcctcaggtcccctgggagcatgccttcccagatctatacagaccctgggaaacatacaacacccgaagggccatttaaatgtgttatgcaggcttctcctgtttctgaagatgcccagcctgacctgtgctaatgcagctctctgctgatgatcacttaaaccccacaggaccttctctttgcatcttttggatagattacttgttcaaccttaaactgtctgctctttctacctctgtctaccgcatggttcagcatttaaagattcttatcataagattcagccacctccttcatgaatccttaaggactgtccctaagactcttcccaacatctctctttctattctgtggactatgatagaacattccagtgcttacttctttagttgtactgctttatgagcaagtctgcaatgaaaaacaatggtggaaaaatgctgacttccctaccttttcattctggggcaattattagtccagtgggccgtctgtatccgtgggttctgtgtctgtgaattcaatcaactgaagatccaaaatattctttaaaaattgcattaatatctgtacaggtcacagagaccttgctgataaaacagcatgtggtaaagaagccagccaaatcccaccaaaaccaagatggccacgaacgtgacctctgattgtcctcattgctcattatactgtaattataatgcattagcatgctaaaagacactcccaccagtgccgtgacagtttgcaaatgccatggcaacatcaggaagttaccccgtatgctctaaaaaggagaggaaccctcagttcctggaattgctcacccctttcctggaaaactcatgaataatctaccccttattattcatgagtttagcacataatcaagaaataaacataaaaatagccaagcagcaacccttgggcctgctctgcctatggagttgccattcttttattcctttactttcttaataaacttgctttcacttaaaaaaaaaggtatctctactgaatatgtacagacttttccccttgttgctattccctaaataatataacaactatttacatggcatttatgttacgttaggtattataaataatctagagttgatttaaaatatatgggaagatgtgcatagttacatgcaaatactacaccattttatatcattaccttgaacatcctcagatgttggtatccaagggttgtcctggaaccaatttcccgtgatactggggatgactatactatcttcctttgctgaagaaaaagtgaaaactagatttgaacaggaaaaagagtaaacaacctatgacatgctgaagctccatgcatttgagtttctgtgtgtactgtggttgttttatactgcttgtttgaactctcatactgtactgttcctgtgttgccgtactcaaataggtttactaacctatctctctagcaaggtcctctctcttgttgatttgaaagtagagtggttgcataggtggtagtgaataagggacagctgggtctttgcacatttgctgcagaccattagagcttgtctttaaagatcatatggttcaggtaacacttttactctagaggacacttagctcctgagcagctaaatagttttcccagagttataacctgtgagaatccctggtcttttgtctctcagtccagcattttggtcactacatctacctgccacgctaatttcattcacttgatctctgtccatcaaaatacacacacacacacacacagacacacacacacacaccctttggaagatagcattttagacaagagtaccaccttttgagctagtaacacctaggtttgaattctactcaacactaactagttatatgacttaattaattcatccttctaagctgtatattttcctcatctgtaaattgaagacaatagttcataaagttgttgtaaggattaaataatttaatgcatatgtaaactgcttaacaaattgcgtagtacctaagtgtcctgtaaaatctctctctctactcatatgtgcaaggcactgagccaggtcaaagagtcaatgcaaacaggtacttcttatttaaataggaaagcatcatacttcttattgtatttcttagacaatttcgcttaacatcgctctagttatattacataggttatatactatattggtttgagatccttaaagaagagagaagtttattttttttctcagataaaagtgtgaggtaagcattccaaattggtaggtgctctgctccacaaatcattcagggatccagggtccttctgaagttgtgctctgccacctagtagggcattgttgatgtctggggtatggttggtgggaaggaaggagagtgtgaaggaggcaaacttactctcttcaggtgtgggccctgagttgcacatcacttccactaacatttcattggcaaaaacatagtcacagggccatatctaactgcaaaagactagaaaactagggcaggtaggcaacccatgaccagattagggttttgttgttacagaaaaaggggagaatgggtttcaatggacaactagcaagctttgccttagagagccaacactgatgaggggttgtgtggcatggtagaaggaattagtcaggacctggtttctgactctacttcagcccgaggcttgcattatgaccttggccacatgcctaaccccttgcacctcagtttccttagtatgaaggaagacctgggactgctcatctccaagatttcttctagctgtatgaagccctcactgatcaccttgtctcaatggttccctttctcctcctaaagcatctcttcactgtgcatctcatgggacataactaatacaactttatttgctaagagcttttatttcctttcattaaactatgggctttgtaaaggaacaagtatatcatagcattttgaaggtatcagaagaagatcaaacagtatatgtaagttggttgattggttggttggtgaatatgaataatttggaactcatttgataaagatactatagatatgaagtactgcagaatgattttctttgggaatgaagcagagctgagagaataacctctaagatctgttctgtgctcaaattttatgattctatgattcagcagctcttcttaagttgggagaaatcccatcttggtttcctatggtggcactccctctaaaaaattatcaccaagttgaacttctggataaaagtttccgtctcctccagtggcttctgcaaggtcaatttaatacacattaaaaggaaatattctcacctctacgaatagagctttatttttaaatcctttagcctttcaggcaagggtctgagtacttacaggatattaaaatgcaggaccatgtagtttgctgagcttttgaatcatattttgtacatttaaatcctaaaacaaatcccttagagattcactttaaaacagtgtattaagcatttattctataaaaatccaagtccctgttttgaagtcagcctccatcaagtttgttcctggtaggagtgactaagtaccaaataagtacagcagtgcttctccaaattctgttgagcatttgagtcatctgggcatctgattaaagtgcagattctgattcaggaggtgtagggtggggggtgactgagattctgcattttaacaagctcccagatgattccctgatgctgctggtctgtggactgagctttgagcaaggatgtaaggaatatttcccatttacatgacacttaaagatgttctgctactagctgattaaatatagggtaattatgttgcctcatattacagtgatttgtatctgttatctttttaagactgtaagatcctgacacccaggaaagagccctatttattctacctcatcttctcctataaatttagcacactttcgtgtactccataagcctttgaaactaattgttaaatggtagaaaagtctaactgaatgctgtttcattccacgtggtgagtagaggttatatacagctatctgtcctgggacatatttgttgctttctgtctcagaattaatctatgagaggaaatgtggccccatagagaatcaaattggaaatactttttcttagcgctttattttcctgtctataatagagactattgacatacacattagcaaatggcttgaatagtcctcagggcttggggcagtcactgcagctattagagtgaagttatgaaagtttacatggctttctttatttagcagtggcagcgtagtgtgtgcctttcttgaaatccaggtgtaagggagagggtgactccatggtctgaaccaggacaggtgggaaatcatgagcatatttggttgccctgaaccagacccaaatgaaattaaatggtgaggtatggaacattattattattattattattattattttttttttttttttgagacagagtccttctcttgcacacactggagtggcacgatcttggctcactgcaatctctgcctcccaggttcaagtgattcttctggcattacaggtgcctgccatcaagcccagctattttttgtatgtttagtagagatagggtttcaccatgttggccaggctggtctcgaactcctgacttcaggtaatctgcctgcctcagcctgccaaagtgttgggattacagtcatgagccactgtgcctggcctggaacattatattttatatccaaattttcaatgcaacgtagcactttgtgcataaaccatcacatacaaattgtatgtactactgcaggccttggtggtttcactggcaagtgacattaccacataattgcttttggaagaagaaacgatgtccttggatattatgtagttaagaaaccgtatagatataatcttgcaggcagctaaatcttcactgcctattcagtatgacttcaaagcctttcccatcactcagctggccaacatagaaactgaaaggagtggaaagtggccgtggtaatgccaaagaatctgtgtgttaccagtctccatagagaagtgggaggtttatgtgatcattcacacaaaacctgcttagctccattccccacttcacaggcagagtcacagactgtccttgccatcactcatgtgctctacttgaccctgagctctgggcccaacagatcagggtttgatttttggccccaccacttgtgtaagttattttgcttttctctgtgttagttttctcatgtgtaaaatggggatagtagattaaatacttttcacagagtctttgagacgtaccaagggcgctaaatctaaatattattttcttaacatcccaactcagagaagctaaatctttttgtctctctagagcttaaaatccagcatgtgatagaatatttataagcactgtatggtatagaaaagtgaaaaacatgacgtaaagacccatgtgtggttgtcactacggaatggggttgtgactgacgtgacctgtagcacatgtgaacattctccactaacaaaagttatggaggcgaggcgaggatccttaggtgaggatcatgcaatgccagagaagacagtgtgctcccagacacagggactgccctcgtggtaactgcatcttagttggggtggggaagggacagacattcatcaagtaatccccagtctttaagttatgacaagttctataaaggatattaggtggctagagatcttctaccaggggccttggcctgatgggggtcagagaaggcttccctgggaatgtgtttatgaaccgagatatatagagaagggaagcaggggctgaggaagggaattgatctatgaagggtgggggcaggaagaagtttttgaagacttcagagaaggccactgtgcctggagccagcaggaagtgcactgggggctggtgaaccaacagggaccaaaccctgcaagaccctgctgtgcagggtgagggtttgggattttatgctgtgagcaatggggtgccgtggaatagcacatgggtgccatggaatggcacataaaataggatagaatagcacatatgctaatgttctccttgaattttaaaggcatcactccggctgcaggatgtcttgtaaggactgctatataaagagtggaagtggagtgaagaagtaacaagaatagatgtgaggaagccagtgaggagcttgtcacaatagactgggcaacaggaaagatggcttttactgggacaggaagaagagtggatggattcaagagtaatttaggaggtaggatcaaaagatcttggtggccaactggatcttgagagcgagggaagggtagtgttaaaggggactcctagtttctggatgatgcaggtgtgcaggtaggggtgttctccctgagatgagagagagcagggcagaacctgagttaggggaagagtaggggtggggagagggagcaatcacgagttcagttttggttatgttgagtttgaggtacatgagacactgaaggagagaaatgtctggagctcaaaggagaggcagagctggaactgtgagctttgcagccagcattggatggtaacgatgccatgggagtcggtgatgatgtctagggtgcgaggtcagagggacaaggataatggcttggaactggaccctgaggaactactataattcacaggtgaagtagagtaagtcagaccctccaaagaagctgagaaggagcagccagagaggtgaggggaaacgtggtgtcacagaagccaatggaaaggactgtttcaatatagagggagtggtcgactgagggaatgcacccagaggcaaaccaacacaaacggacagctcggaaaataaaaagctctgaagtcaggaggcagttcgaatctagtatttttagaatgttcccatgaaacccgctcttccttagcttcctccattgtaaaatgggatagcagcagagttcttttgagaactgtgtgagatgtgtgtaagatacttggcacagagctggcacccagctactggggtccagcaaatggtagctggtataataatgagcacaggtgtggtggaggaaatgagaataagaggaacaggggcatgggggtgcgcaggacacagtccctggtctcactggagctcagcgtttgtagctgggaagatgaaaaagtttaagtgaatccgaaggagggaccttggatgctgggccagcaggggttgcaaattctgggcctaatctggttgggagatgaccagggcctagagtaagggcagggcagggaggggaaggcagagaccctttgggtaatttatcaggactttggaagtggtctggaacagaatcgagaggttgaaagttgtttttagcattcaagcttagaatagggttttctctttctttctgtatatttgagttgttaactagataatcaggcctagctgtcttttaaagagtgctctatgaaactcagttggggaagcagcatgccactgtcaccttgttcttcctgatgcaaagtgaagactggacactaagcttggagcctggagggacctccagaagtcaaggagagagttcatataagaccgttcaatttgaagaaatagaaattaaataaaaaatgacttcagtctagacgatgccctggaatgaaaaggacaaggatggtttggtgtaacagataatgacttattttattaaaagttaaggagagagaggcaaagagttgtctctaccctgtaaaggttctctgaatgaatgtattggttattcaagtgcaagtacttattttctttggtggaactattccttttgtaattttggctgggctgacagttccatcagttgaacattcagtagtcctggcctagaccagtggtgtgccatgaagtgtgcacattagaatgagtgagtgtgggtcttacctcagaggtttgtgtcatctgttagattttctagcctccccattcttaggagcctgaagctgctgagaaaagatgaatctatttactctggaggggcagcttggctgatgtggtattttctgaagtcatcagctggccctagaaattgatgtggttaatttcattgttatcctctgtgctgctcttcaaaccatttttacctgctaagttgccacagattttgtatcgttgatgatcattggcaagaacataagaacaaatagtagatttgttaaccccctcccccaaatagatccttgaatctcgtatgggtgcctgatcaaccatagttgcattccccaaaaaagttatcacctgacattacagtgtagaaagaataataaatagggaatctttcctttaattgtagaattttccttaaataatgtctttcatccgattttatttctatttgtgacataatgtcacatactgttgataccattaagatgtggaagagtttaatgccagacactctatctggcaaacaagaacaggtggttgccaggagtcgaggggagggaaaattgcagagctgttgtttaatggatatagcgtttcagttttgtaacatgaaaaagttctggctatttgttgctgcacaacaatgcatatacttaacattgttgacctgcacactaaagaatggtgaagatggtaaattttacataaatgttatgtgttttttaaaccacaattaaaaaaaaaaaccaagagcaataaaacagaaaaaaagaggcaaacctcaacataaggcaagactgagtcaacagctcagctcttccttaccttcaggcctattgctctgggaactgtcacagatcttcatgtctgtgctttgtttccttgtgttcccccttgatcaattcctcctataggagaatgtagtcactcacatgggtgattggtttgccactgactatcaccaggtcgaatgggtagagagagaggaaggcatgaatttgagtagtgtaaggatagggctttgaggactgtggtcctccttaggctggtgtggcctgaactgcgaggaaagcagccagaagttctactccagcgtttaaggacctatcaggagggtctctttgtccctgcccccttccacagcactgcacctgcagggctagagggtgggttgccctaacagcatttgctttcccagttgaaagagcaacgcacagaggtaaaagccattgggaaaatgcttcacttgaagccagcagttagaaggtgaatgtcatgccatgggacctcagatgtgcaccatagacaaattactgagaaatccaggtttgattcagttctttcaattctctatgtgagcacctagatggagaaaaaaatcttgaccaaaaatgcttctgtccattcacactgtaagccaaaggaacattgaatcataaagggaataaactgggatttaggattcaatagttcagacacacaaaaaagaagtgttgttctttttctgagcagtagtggaggtgcccgggttgtgagaaggatgagcttactgtgtcacaccatcatgcctccaagtcattcattttaacagtccagggagtgaagtgagttctctccctgagtttcagtagcagtaaaagttctccctaattaatgcctttgtttattttattttattttatttttttttgtgacagccacctctgtcatcatgggcacttaaccacttgtcttatattaattctcatgagggcttccctgttggactttaagctccttgaaggcaggacctgggtcataatcatctctgcatccatccttcagcaagtttgcagaagcttaatcaaaggttgattgaatcaccccagagaaattacatgggctgttcaggttcaaaacatttcattcttctggctgtaattctttgtttttactttgtaacctaaaatacatgttacttttggtgctgaaactatcatgcataaaactgactgctgacacagacaggaagacagtatctttgaaaataatgtgcaattgagaatctatgattatgacattttttatacttcagaaataagtatatcttgcagcagtgaatcatctatattctgtggcacagtttttctttgatttttcttttttttttttttttgggacggagtcttgctctgttgccccagctggagtgcagtggtgccatctcggctcactgcaagctccgcctcctgggttcacaccgttctcctgcctcagcctccctagtatctgggactacaggtgcccgccaccacgcctggctaattttttttgtgtttttaatagagatggggtttcaccatgttagccaggatggtctcgatctcctgacctcatgatccgccctccttggcctcccaaagtgctgggattacaggcatgagccacggtgcccagccagtttttctttcatttttcttaaatttgtttctctcatcagtgaggcttaatgtattttgatcagaagttcagtaggttcgttttcccatttatgaaataaagagtttaattatgggtgcaaagcattttcaagttgcttttgagtgactttctcacttcacactcaatgtcagtcactcacagagattattttccttggctctcccttgtctttctaggtcatatgtctcgctcttgttttgccccttgcttatactcacattctttgtttttttccttcctgttttcaacatttttctcatgctatttttcttggttgttgttcccttcaaagaaaattgctcttctattagaaaaagtaccaagttctctccagaagcagacaagcataaagtgctgactgccaaggtgaacgctcagcaatttgaagggctggagaaagccgcactggtcagtgttacgggaatgcctcttatcatcaccacacaaatccagttctccttttaggcagcctgctattaattacatccaaatcagcttttcaaaaagcaaatgccgttcattaaagtcgcacaaccatttaatctttttgcttcatctgtgattgagtccatagcaaccagatgtagcttgatgcctttatatttttagaataaaagaagtaagttagtaaagatcatttataggcctggcagatgagaagataaaagatgcagttgtctttgataaggggtcttgggtttttcccgttataagcttttgtgactttaactctccagagattccagtgtcacctttacctcttttgtgagaagtccttttgggcaacatgtatgagttcatactcagttaatgagaattcctttttgcaggattccactggagctaaattggtaactttaacagtttcctccctatcatacccaaggctgcaggcattttcctgttctgcttagagaatatttctaagctctgcttcactctgcttcagacttgctcacttgcctggtttccttggtttctgttctctgccatctacccagatccttctactttacctggcttttctgggtggatgcttatttggtggtttgcttgacttatgacctggggacttggcgggtccctaaggctttttttcctattcctatgcagtgcaacctcccatcctttacccaagctccttaattactgcaccttaaaccaacctggattcattgcctcagattcttacttcttggagcagggaggagtcagactctatcattaattttggattgtgactacctcatctgaaaagtatatgtggctaggaggagagcacaggctctgcagccagtctccttgggttcaaatcccaggtctgccacttataagctgtgtgattttgggcaagaagtttaacctcttttaccccagttctctcatctgtgcagtggagataatggctgtacctaccttacagggttgaaatgaagagtaaaaaattactctatgtaaagggcttagaacagtgtgtctgctacacagttaacactatatgttttaactgttactaagacattaaaatccacattcactaaactctgactgccagcaataaggcagatggattagatcaatgcttatcagagggggatccaaacgtttactgtgagaaaatttaaatcttgtgctctcatttggatgaaaatgtaaatacatcaagataagcatatttttggattatttggacaacttttcatagctgataatcccacgttattagaaagcctgcatttgcattgtgtttacggtcgtattattatgcaatgatgaccatgatattgaggctggctgcaaggaaggcctagctatggtcttagagaaaagagtggagggggaatagagtctcttccagtacatgtaatctgagcctgtcaaactgttagagaccagctctgtagcagtcagtacatagccacattttagtgactggtgcactaaaacaaatttgggttttattaggattttacttaatttaggaatttgttttggttttatggtaatataaaagagctatcaggattcggagttttatgtaggcttacggttacatttatttaagtaacattataataaaaataatttgttagcactggagggccataagaatttttatctcataaaaggagtctgtccagatatttctcaaacctgggcaagatgagaccactctgtgcttcccaaatgccagtatatgaattgtgctaagaaggtggtaaaagaaagtcccttgggaagtttgttaaaaatacagacacttaggctttatcctgtagttgctgagctgggccagtgaacctttatttttgctgagttgctcaggtaattttgctaagcaacaggtttgggctctttgaagtagatggtctttcagtgttcttcctagccctgggattttaggattccgtgtcttcatcttcgaaattgacataatggtacttgtgcctttcttactgcacaagggagtagcaagcattaatgaggcaatttttgtaagtgttcttggaagaaaagtacaatttaaatataatatattgaggccagaattgaactagatcatctcagcacgcaaattgtcccctcagaacaaagaggtgactcaggtcattagaacatctcagcaagttcttgattactgctaccagggatttagtttccaaacgtattggtccatctctagatggaatcaagccctgtgaaacctggtccattaccgtgggatttacaaattagcaacaaggctggacaggaatgtgcataatgaggcaccctgtgcttattgtattatactactttccttgggctgccataacaaaagaccataaactgggtggcttacaagagaaatgtgttctctcccagttctggtggctggaagtaaaaagccaggtgtcagcagggttggttccttcttgaggctgagggagagcctgtttcatgcttccttcctaatttctggctgtgcctggtgatacttggtgtttcttgacttgtagattgctcactctaatctctgccttccttgttaatggctgtcttcttcttgtgtgtctctgtgttttcacatgggattcttctctgtgagtcttgttctctcttcatcttcttataagaacatcagtcatattggattaggggcatatcctactccagtatgacctcattttaatttaattatatctgtgaccaccctaattccaattgggtcacattctgagggactgagggttaagccatcaacatatatttttaggggacaaaattcaactcacaagaccgtgtttctttttttttttttattatactttaagttctagggtacatgtgcagaacgtgcaggtttgttacatatgtatacatgtgccatgttggtgtgccgcacccattaactcgtcatttacattaggtatatctcctaatgctatccctccccactcccctcaccccacaacagggcccggtgtgtgatgtcccccttcctgtgtccaggtgttctcattgttcaattcccacctatgagtgagaacatgtgatgtttggttttttgtccttgcgatagttggctgagaatgatggtttccagcttcatccatgtccctacaaagcacatgaactcatcattttttatggctgcatagtattccatggtgtatatgtgccacattgtcttaatccagtctatcattattggacatttgggttggttccaggtctttgctattgtgagtagtgccacaatcaacatacatgtgcatgtgtctttatagcagcatgatttataatcctttgggtatatacccagtaatgggatggctgggtcaaatggtatttctaattctggatccctgaggaatcgccatactgtcttccacaatggttgaaccagtttacagtcccaccaacagtgtaaaagtgttcctatttctccacatcctctccagcacctgttgtttcctggctttttaatgatcaccattctaactggtgtgagatggtatctcattgtggttttgatttgcatttctctgatggccagtgacgatgagcattttttcatgtgtctgttggctgtataaatgtcttcttttgagaagtgtctgttcatatccttctcccactttttgatggggttgtttttttcttgtaaatttgtttgagttctttgtagattctggatattagccctttatcggatgagtagattgcaaaacttttctcccattttataggttgcctgttcactctgatggtagtttcttttgctttgcagaagctctttagtttaattagatcccatttgtcaattttggcttttgttgtcattgcttttggtgttttagacatgaagtcattgcccatgcccatgtcctgaatggtattgcctaggttttcttgtagggtttttatggttttaggtttaacatttaagtctttaatccatcttgaattaatttttgtataaggtgtaaggaatggatccagtttcagctttctacatatggctagccagttttcccagcaccatttgttaaatagggaatcttttccccatttcttgtttttgtcaggtttgtcaaagatcaggtggttgtagatgtgtggtattatttctgagggctctattctgttccattggtctatatctctgttttggtaccagtaccgtgctgctttggttactgtagccttgtagtatagtttgaagtcaggtagtgtgatgcctccagctttgttcttttggcttaggattgatttggcaatgcgggctcttttttggttccatatgaactttaaagtagttttttccaattctgtgaagaaagtcattggtagcttgatggggatggcattgaatctataaattaccttgggcagtatggccattttcactatattgattcttcctatccatgagcatggaatgttcttccatttgtttgtatcctcttttatttcattgagcagtggtttgtagttctccttgaactccttgaagaggtccttcacatcccttgtaagttggattcctaggtatttcattctctttgaagcaattgtgaatgggagttcactcatgatttggctctctgtctgtctgttattggtgtataagaatgcttgtgatttttgcacattgattttgtatcctaacactttgctgaagttgcctatcagcttaaggagattttgggctgagacgatggggttttctagatatacaatcatgtcaactgcaaacagggacaattggacttcctcttttcctaattgaataccctttatttccttctcctgcctaattgccctggccagaacttccaacactatgtcgaataggagtggtgagagaggacatccctgtcttgtgccagttttcaaagggaatgcttccagtttttgcccattcagtatgatattggctgtggttttgtcataaatagctcttattattttgagatacgtcccatcaatacctaattgattgagagtttttagcatgaagcgttgttgaattttgtcaaaggccttttctgcatctattgagataatcatgtggtttttgtcattggttctgtttatatgctggattacatttattgatttgcatctgttgaaccagccttgcatcccagggatgaagcccacttgatcatggtgggtaagctttttgatgtgctgctggattcagtttaccagtattttattgaggatttttgcatcgatgttcttcagggatattggtctaaaattctatttttttgtcgtgtctctgccaggctttggtatcagtatgatgctggcctcataaaatgagttagggaggattccctctttttctattgattggaatagtttcagaaggaatggtaccacctcctccttgtacctgtggtagaattcggctgtgaatccttctggtcctggactttttttggttggtaagctattaattattgcctcaatttcagagcctgtaatagagaagttgtctctattcagagattcaagttcttcctggtttagtcttgggagagtgtatgtgtctaggaatttatccatttcttctagattttctagtttatttgtgtagaggtgtttattgtattctctgatggtagtttgtatttctgtgggatcggtggtgataccccctttatcattttttattgcatctatttgattcttctctcttttattctttattagtcttgctagtgtcaattttgttgatatgttcaaaaaaccagctcctggagtcattgattttttgaaggtttttttgtgtctctatctccttcagttctgctctgatcttagttatttcttgccttctgctagcttttgaatgtgtttgctcttgcttctctagttcttttaattgtgatgttagggtgtcaattttagatctttcctgctttctcttgtgggcatttagtgctataaatttccctctacacactgctttgaatgtgtcccagagattctggtatgttgtgtctttgttctcattggttttaaagaacatctttatttctgccttcatttcgttatgtacccagtagtcactcaggagcagtttgttcagtttccatgtagttgagcagttttgagtgagtttcttaatcctgagttctagtttgattgcattgtggtctgagagacagtttgttataatttctgtccttttacatttgctgaggagtgttttatttccaactatgtggtcaattttggaagaataaatgtgatgtggtgctgagaagaatgtatattctgttgacttggggtggagagttctgtagatgtctattaggtctgcttggtgcagagctgagttcaattcctggatatccttgttaactttctgtctcgttggtctgtctaatgttgacagtggggtgttaaagtctcccattattattgtgtgggagtctaagtctctttgtaggtctctaaggacttgctttatgaatctagatgctcctgtattgggtgcatatatatttaagatagttacctcttcttgttgaattgatccttttaccattatgtaatggccttctttgtctcttttgatctttgttggtttaaagtctgttttatccgagactaggattgcaacccctgctctttttttgttttccatttgcttggtagatcttcctccatccctttattttgatcctatgtgtgtctctgcacatgagatggatttcctgaatacagcacactgatgggtcttgactctttatccaatttgccagtctgtgtcttttaattggagcatttagcccatttacatttaaggttaatattgttatgtgtgaatttgatcctgtcattatgatgttagctggttattttgcttgttagttgatgcagtttcttcctagcatcgatggtctttacaatttggcatatttttgcagtggctggtaccggttgttcctttccatgtttagtgttcccttcaggagctcttgtagggcaggcctggtgttgagaaaatctctcagcatttgcttgtctgtaaagtattttatttctccttcacttaggaagcttagtttggctggatatgaaattctgggttgaaaatctcctggtgtgccgtttgctaagaccgttggaaaagtgcagtattggggtgggagtgactcgattttccaggtgccatttgtcaccccttcccttggctaggaaagggaattctctgaccccttacgcttcccgggtgaggcgatgcctcgccctgctttggctcatgctcagtgtgctgcacccactgtcctgcagccactttccgacactccccatggagatgaacccagtacctcagttggaaatgcagaaatcacccgtcttctgcgtcgctcacgctgggagctgtagactggagctgttcctgttcagccatcttggaaccgccccctgaccatgtttcattttacttagaggaacgtaggatactttacatttctgaatgagttctttttccaaaaattgatttgaaatgcagagggcattctcccttagaaaccatcttctatgtcatagtgaggtttctggggcagcccataacaactgatttagttcacaatatactggaagtagaatttctgttaagggtcctatagaacctaaacaatgcaccagacaaaatcttggtgggaaaatatattccaggtttcaatttagggtgcttatctgacccagcaattccattactgggtatatacccaaaggaatgtatatcattctatcataaagacacaagtacatgtatgttcattgcagcactattcacaataggaaagacatggaagccacctaaatgcccatcagtggtagactgggtaaagaaaatatgctacatatacaccatggaatactatgcagctataaagaagaacaagataatgtcctttgcaggagcatggatggagttgcaggccattatcctaagcaaactaacacaggaacagaaaaccaaatcctgcatattctcacttataagtgggagctaaatgatgagaacacatggacacatggcggggaacaacagacactggggcctgtttaagggtggagggtgggaggagggagaggtttagaacaaacaacgactgggtactaagtttaatacctgagtgacaatctgtacaccaaacccctgtgacacgagtttacctatataacaaacctgctcatgtacccttgaacccaaagtaaaagttaaaaaaaaaatcaatttagggtgctaagaacccaatttttctcatcagctccactggctctggtgggtcttagaaacttcatacttgtgttttttctttcttggttcttctgtgttcatttacatttccttttcgttgttgttgtttttgttccctgttttggttctagtattactttttatgttattgttgtacctgaaatcccttgtggaaggagtggagagtagggaagggaaatgctctgtgctgtggacagtctttgtcccactttcctattaaaatttacttgtcgtgattcctgattttttcagcatcattgctgcagttctacatgatacccagctatccagtatcttaatttctcttttgtgtatgaggagttctaatgcatttacttcataagccatagtgttgagtcagaggggctctcaggaatgagaagcaggaaattctagtaggttaactttgtgtatccttcacatcatattgtttaaacagtttttgtgctggctgtgtgtctcagccctgttcttttactccaggctccttaatgagccactgctgtcagttcagggaggctaatccctggacttaaccctttgtggcccttcctttaaatggcaaagagttaatgggaaagattttcttttttaaaaataatctctgcaaagtcttttcctgaaatcaagtggtagatattggtaaagtgttgtaaaaatgcaacgtgttcaaatatcttcagaatatccattctgaatcaacatgggtaaataggcgtttctcttttccttaattcaaagggattgtgttagttcaggtcctctgataaacagaggcaagatgggattacttgtggcagagatttatagggagaaacacctggaaaggataaagagaagggaataggagtaggtggggagagctctcagattgcgatgctagactgactgctgtgaaaggaggactgagagaaagactcaaatgatagttcagttctgagaatgtttttgccaggctgcttgtgagttctggagccgaagtggcccacttgaggactcctgtgtcccacaggaatgagtctgcacaggtgccctgctgtgctcagacattgcctgggagcagtccaggggaagcgtggacactgcacaaatgtggtagtggctccaggggtggtggtgggtgcctgggactgtcagttacctatgcaccccaacgacagaagagctacgcagtgcatttccatgactttcttagggatctctctaggggtccatgaactctaagggtaaaaattgcatccttattttcagtaacttctaactgaattttagtggtttgtctaattatgagtgtagccagtagccataatgcagtatatgtgtctttgtcaccaataaatattacagataatttcatatcacattatagttgtcacagataactcaaaatgtcatttatgctcatcactactttgaaactatagtagttattaaaccggccactatatctttttatttaatcagatagtacaaagacatatttactattgtatcacaaatttaaaagataacttgataatggaatttaagtatttctttttttttttttaatttttttttattatactttaaattttagggtacatgtgcacattgtgcaggttagttacatatgtatacatgtgccatgctggtgcgctgcacccactaacgtgtcatctagcattaggtatatctcccagtgctatccctcccccctcccccgaccccaccacagtccccagagtgtgatattccccttcctgtgtccatgtgatctcattgttcaattcccacctatgagtgagaatatgcggtgtttggttttttgttcttgcgatagtttactgagaatgatggtttccagcttcatccatgtccctacaaaggacatgaactcatcattttttatggctgcatagtattccatggtgtatatgtgccacattttcttaatccagtctatcattgttggacatttgggttggttccaagtctttgctattgtgaataatgccgcaataaacatacgtgtgcatgtgtctttatagcagcatgatttatagtcctttgggtatatacccagtaatgggatggctgggtcaaatggtatttctagttctagatccctgaggaatcgccacactgacttccacaatggttgaactagtttacagtcccaccaacagtgtaaaagtgttcctatttctccacatcctctccagcacctgttgtttcctgactttttaatgattgccattctaactggtgtgagatgatatctcatagtggttttgatttgcatttctctgatggccattgatgatgagcattttttcatgtgttttttggctgcataaatgtcttcttttgagaagtgtctgttcatgtccttcgcccactttttgatggggttgtttgtttttttcttgtaaatttgtttgagttcattgtagattctggatattagccctttgtcagatgagtaggttgcgaaaattttctcccatgttgtaggttgcctgttcactctgatggtagtttcttttgctgtgcagaagctctttagtttatttagatcccatttgtcaattttgtcttttgttgccattgcttttggtgttttggacatgaagtccttgcccacgcctatgtcctgaatggtaatgcctaggttttcttctagggtttttatggttttaggtctaacgtttaaatctttaatccatcttgaattgatttttgtataaggtgtaaggaagggatccagtttcagctttctacatatggctagccagttttcccagcaccatttattaaatagggaatcctttccccattgcttgtttttctcaggtttgtcaaagatcagatagttgtagatatgcggcgttatttctgagggctctgttctgttccattgatctatatctctgttttggtaccagtaccatgctgttttggttactgtagccttgtagtatagtttgaagtcaggtagtgtgatgcctccagctttgttcttttggcttaggattgacttggcgatgcaggctcttttttggttccatatgaactttaaagtagttttttccaattctgtgaagaaagtcattggtagcttgatggggatggcattgaatctgtaaattaccttgggcagtatggccgttttcacgatattgattcttcctacccatgagcatggaacgttcttccatttgtttgtgtcctcttttatttccttgagcagtgggttgtagttctccttgaagaggtccttcacatccctttaagttggattcctaggtattttattctctttgaagcaattgtgaatgggagttcactcatgatttggctctctgtttgtctgttgttggtgtataagaatgcttgtgatttttgtacattgattttgtatcctgagactttgctgaagttgcttatcagcttaaggagattttgggctgagacaatggggttttctagataaacagtcatgtcatctgcaaacagggacaatttgacttcctcttttcctaattgaataccctttatttccttctcctgcctgattgccctggccagaacttccaacactatgttgaataggagcggtgagagagggcatccctgtcttgtgccagttttcaaagggaatgcttccagtttttgcccattcagtatgatattggctgtgggtttgtcatagatagctcttattattttgaaatacgtcccatcaatacctaatttattgagagtttttagcatgaagggttgttgaattttgtcaaaggcgttttctgcatctattgagataatcatgtggtttttgtctttggctctgtttatatgctggattacatttattgatttgcgtatattgaaccagccttgcatcccagggatgaagcccacttgatcatggtggataagctttttgatgtgctgctggattcggtttgccggtattttattgaggatttttgcatcaatgttcatcaaggatattggtctaaaattctcttttttggttgtgtctctgcccggctttggtatcagaatgatgctggcctcataaaatgagttagggaggattccctctttttctgttaattggaatagtttcagaaggaatggtaccagttcctccttgtacctctggtagaattcggctatgaatccatctggtcctggactctttttggttggtaaactattgattattgccacaatttcagagcccgttattggtctattcagagattcaacttcttcctggtttagtcttgggagagtgtatgtgttgaggaatttatccatttcttctagattttctagtttatttgcgtagaggtgtttgtagtattctctgatggtagtttgtatttctgtgggatcggtggtgatatcccctttatcattttttattgtgtctatttgattcttctctctttttttctttattagtcttgctagcggtctatcaattttgttgatcctttcaaaaaaccagctcctggattcattgattttttgaagggttttttgtgtctctatttccttcagttctgctctgattttagttatttcttgccttctgctagcttttgaatgtgtttgctcttgcttttctagttcttttaattgtgatgttagggtgttaattttggatctttcctgctttctcttgtaggcatttagtgctataaatttccctctacacactgctttgaatgcgtcccagagattctggtatgtggtgtctttgttctcgttggtttcaaagaacatctttatttctgccttcattttgttatgtacccagtagtcattcaggagcaggttgttcagtttccatgtagttgagcggctttgagtgagattcttaatcctgagttctagtttgattgcactgtggtctgagagatagtttgttataatttctgttcttttacatttgctgaggagagctttacttccaactatgtggtcaattttggaataggtgtggtgctgaaaaaaatgtatattctgttgatttggggtggagagttctgtagatgtctattaggtccgcttggtgcagagctgagttaaattcctgggtatccttgttgactttctgtctcatggatctgtctaatgttgacagtggggtgttaaagtctcccattattaatgtgtgggagtctaagtctctttgtaggtcactcaggacttgctttatgaatctgggtgctcctgtattgggtgcataaatatttaggatagttagctcctcttgttgaattgatccctttaccattatgtaatggccttctttgtctcttttgatctttgttggtttaaagtctgttttatcagagactaggattgcaacccctgcctttttttgttttccattggcttggtagatcttcctccatccttttattttgagcctatgtgtgtctctgcacgtgagatgggtttcctgaatacagcacactgatgggtcttgactctttatccaacttgccagtctgtgtcttttaattgcagaatttagtccatttatatttaaagttaatattgttatgtgtgaatttgatcctgtcattatgatgttaggtggtgattttgcttgttagttgatgcagtttcttcctagtctcgatggtctttacattttggcatgattttgcagcagctggtaccggttgttcctttccatgtttagcgcttccttcaggagctcttttagggcaggcctggtggtgacaaaatatctcagcatttgcttgtctataaagtattttatttctccttcacttatgaagcttagtttggctggatatgaaattctgggttgaaaattcttttctttaagaatgttgaatattggcccccactctcttctggcttgtagggtttctgccgagagatccgctattagtctgatgggctttcctttgagggtaacccgacctttctctctggctgcccttaacattttttccttcatttcaactttggtgaatctgacaattatgtgtcttggagttgctgttctagaggagtatctttgtggcgttctctgtatttcctgaatctgaacgttggcctgccttgctagattggggaagttctcctggataatatcctgcagagtgttttccaacttggttccattctccacatcactttcaggtacaccaatcagacgtagatttggtcttttcacatagtcccatatttcttggaggctttgctcatttctttttattcttttttctctaaacttcccttcttgcttcatttcattcatttcatcttccattgctgataccctttcttccagttgatcgcatcggctcctgaggcttctgtattcttcacgtagttctcgagccttggttttcagctccatcagctcctttaagcacttctctgtattggttattctagttatacattcttctaaatttttttcaaagttttcaacttctttgcctttggtttgaatgtcctcccgtagctcagagtaatttgatcgtctgaagccttcttctctcagctcgtcaaaatcattctccatccagctttgttccgttgctggtgaggaactgcgttcctttggaggaggagaggcgctctgcgttttagagtttccagtttttctgttctgttttttccccatctttgtggttttatctacttttggtctttgatgatggtgatgtacagatgggttttcggtgtagatgtcctttctggttgttagttttccttctaacagacaggaccctcagctgcaggtctgttggaataccctgccgtgtgaggtgtcagtgtgcccctgctgggggggtgcctcccagttaggctgcttgggggtcaggggtcagggacccacttgaggaggcagtctgcccgttctcagatctccagctgcgtgctgggagaaccactgctctcttcaaagctgtcagacagggacacttaagtctgcagaggttactgctgtctttttgtttgtctgtgccctgcccccagaggtggagcctacagaggcaggcaggcctccttgagctgtggtgggctccacccagttcgaggttcccggctgctttgtttacctaagcaagcctgggcaatggcgggcgcccctcccccagcctcgttgccgccttgcagtttgatcttagactgctgtgctagcaatcagcgagattccgtgggcgtaggaccctccgagccaggtgtggtatatagtctcgtggtgcgccgtttgttaagccggtctgaaaagcgcaatattcaggtgggagtgacctgattttccaggtgcgtccgtcacctctttctttgactcggaaagggaactccctgaccccttgcgcttcccaggtgaggcaatgcctcaccctgcttcggctcgcgcacggtgcgcgcacacactggcctgcgcccactgtctggcactccctagtgagatgaacccggtacctcagatggaaatgcagaaatcacccgtcttctgcgtcgctcacgctgggagctgtagaccagagctgttcctattcggccatcttggctcctcccctaagtatttctttaaatgtttctttagatgtatttttctatgtattttattttatgcatttataaatagtgttctaagtaggggttcatagtcttcattggacagtcagatgggtccatagcacaaaaatgattaggaagccctcctgtagatagtattcaggaagactacattatttatatgagtccaatagacttacccttcattgggctcctaatatatgagaacaacagcctcagttctcattagaatatattatggtagaagatgattcattgatttaaccagtagtgtcttctgcatgccaggccttctctggtgctggagctgcagcaatgagcaaaacacataagtctcatctgggttctccagatgagagatctagtgtagggcccagaacagacctctccgacgaagggccctttaagctgggtctgaagaatgagtgggagtgagccgggagaagaaaaggtgaagacagaaggaagaataagtgtgaaggtttggaagtggggcactcagctcactgtaagaactgaaatcatatagggccttagaaactgagtgaaggtcagactttttcctgagggcagtagaaagcttggaaggggttttaagtagggagtgatgtgatcagactgcctttcaaaatgttgcttggaaagaaactggagacacccttgtggtactaaggtgaaaatgaaggtggcgtggcttggaagtatatgataggaaacaatggcagagagaacttttcaagttacttttgtctgatttctgttgaagatttctttctgactctgtgagaatgactgggccctgaatttcaatttgttagtgtatcatgtattgcattacaattttggaagcctttttcttagagttgagatggccatagtagctgcagttctcgccgaagtctgttgcaagttcctctctgggagtataaaacctcgcacaagtcttggaaaaatatttctctgtaatgttggaccacggagcaagtggctgacttggtcaggctctcttagtgtacatttgtaacttgctgcagtgttaatggcctaacttctgcattaggaagaaaataaacttatcattaataatgtacatgattatattgccaattaaacaaaagctctggctgcctcttcgtaacaaacttgtaaatgggttcaaggtttgtttgttactgggtaaaaacaaatccattttggttgatggctaattcacagccatataggcaactcataaaagtatatgaaaatgagtctctgggataatcagattttatttctagtgtctaatcgatgctcagagctttgggatttaattatgtctgctgaagaaacacaattcaatttctcttcctcccttttaagcctaactgcagtcttcattttaattcataaatacataacactttatagctattttctggcccagtacctggtacctattagatactcaatagaagttggttacattattgaattcctcttctaactgaatgaaaacacatatcaagtaataacctaattctacttctgggtatatatacaaaaaaattgaaagcagagtcacagatagatatttgtacatccatgttcatagtgacattatttatggtagctgcaatgtggaggtaccccgagtgtccattgatgggtgtatggataaacaatatctagaggaatattattcatccataaaaaggaaggaaattctgacatatgcttaaaaaggaagttagttctgaaatatgacagggatgaaccttgaagacattattctaagtgaaacaagcccatccccaaaagacaaatactatataattccacttatgtgaggcatctcaggtagttaaattcatagagacagaatgctgattcccaggggctagcaggaggggagaatggagagttttaaaaaatgggtatagagtttcagtgttacttgatgggaagttctggagatggatggtgctgatgacattatgaatatacggtagttaattccactgaattacatacttgaaaatggttaagatggtcatttctatgtcacgtgtatattaccacgataaaaaaattgggaaatcaacacaggtgtggttttgcttcttttccctccattttttgcctagtggttgactctgtacattttacacttacttcaaagaaataagaagtgagaaattgtttcagtaagaggacttcctatttctggccatctaggctggctggcctgcctgtaatgctgggcagcagggagtgcatcaggtgggtatcacatgggcactgaggcaggatgggtgaagtatgggtgaatggaatatgagttctactctctcccctctctatggcacccacccagcttctaggagggaccacatagcttctttaccccatcttctatcccttagcacattcccttagtgtcttcattcacttagtgtaatgttttcaaggtacataaaaattttattttgtagatgtatcagaactttatttctttttagggctgaataatattccactgtatgtctataccgcattttgtttatcaatcagttgatggacatttgggttatttcctccttttggctgctgtgggtagtgttgctatgaacattcgtgtacacgtatttgtttgaggacctgttttcggttcttgtgagtatatacctaacagtggaattgctgggttatatgtcaatattttatttaactttttgaggagcagctgaggtttttccacagtggctgcatcattttatgttcccaccagcaacgtatgagagttctaacttctctacatcctcactaacacttgttaattttctctttctttttttatggccttcctagtggatatgaagtactacctcactgtggttttgatttgcatttccctaatgcctcatgatgttcagcattttttcatgcctttattggccatttgtataaatgctacagtgtgggttaaccttaagaatattatgctggccgggcacggtggctcacacctgtaatcccagcactttgggaggccgaggtaggcggatcacctgaggtcaggagttcgagaccagcctgaccaacatggagaaaccccgtctctactaaaaatacaaaaattagctgggcatggtgacacatgcctgtaatcccagctactggggaggctgaggcaggagaatcgcttgaacctggggggcggaggttgtggtgagccgagattgcgccattgcactccaacctgggcaacaagagtgaaactccatctggaaaaaaaaaaaaaagttatgctaagtgaaagaagccagaccgaaaggtcatatgttgtatgattcaacttatatgaaatgtccagaataggtaagtccatagagacagaaagtgaatgggtggttgccaggggctggggagtgaggggaatggagagggactgcttttaggtctagggtttttttgtgtgtatgtgtgtatttggagggggggtgttgataaaaatattttagaactagatagaggtaatggttgtacaacattgtgaaaatgcaaaatgtcactgaattgcacactttaaaatggtcaattttatgttatgtgaattttacctcaataaattaaaaaccaaaaacacctggttagaagttgagcctggatttctggttccagcagcatggtggactgagtgactgtggatttctacatcctgctacaaacacagagaagtgctggatacgttacaacaaaatttttaaatatgcaactgatcttggaaggaaatctgttgttgtctgtggaggcaacttgaagcctgagtactggcctcctgcactgcagccatggtgtcctggcagggacactggaccaaacataatgctgggatgtttaacaccaacatggcatcaagagacatggccttggctggtggaggacagggaatgggagctgaaacccctgcacaaagatggcaccctggaaaagctgcccctcagcgaaaggaggtctggaaaagttacttgtcagcaagccaggggaacatcaaaggagcacctctgtttctgaggctctgagtgagagagaataaaagacatgaagccccagccttgacttcttgatgcaagaatccccaactcagagattaatgtgcaaccctgttccaggtgggtgaagctcctggtgtactcagcacaagcaaacaagaaattgttcatttgtagtgacactgctgcaattcagggtgtacaggattcccatgggtaaaacaccctccatgaaaaataagttcacctgaaaaattacagatcacacaaggaaacaaatgttccagcaggagggaaagtaaataaatgtaacaaacaggaggggtagcatcttaagaacctggccaagtggaagcatgtttccaatgactgaggaatgaggaagttgagactggctgtacttcttcatatatagttgaacagttgctaccaggggtgtgtaacatgcttgcttaagggattaaaaaaggggaaagagagtctggcctgttcacctgctatcctataatggtctgtcaagacctttaggttttttcctaaggatcaagagctcaaggaaactttcaagtaatctatttagatggcagaaaagttctgcctttgagcatattaactgtaatgcttttgatgtataagaagctgtacatatttaatgtatacaactcgatgagtttggggacaagtatacacccctgaaactatcaccaccaaagccataaacatatccatcacctcccaaagttgccttttgtcccctttattattattacttgttgtaggaacacttaacatctactgtcttagtggatagaacatgagctaaattttctcagcacacacacacacacaggtaactgtgtagatgttaatagatatgttaattagtttgattgtggtgatcatgtttacctatatcaaaccaccaagtcatatgcctttaaaaaaatattattttaacagagatagcctatgactttcaagacatcacaaagagtcacaccaacctctcagtaccaaaacctaaagggaggtgtcctccctgaacatgtaactaatagatcttatgttctaaattagaaaatgaaacaaaacttcagaggactccatcttcttctcctagccattaccaactcagggttacaagtatcttctttaaataaaactcaaaactgcaacttaaagaaaaagatctacccttttagcagattttaagtgtacaatacagtatcattagctatgggcactatgccgtataatagatctccagaacttatttatcttacataactgaaattgtctactctttagtcatcagtgctcaggtgttcttaaccaaagatttgccttaatctctcttgagtgtgcagcttggtatttgcccattattgtgaggagtgaagattatgaactggtaaatggaacttttggaaaacaaaccctgaagcttccttgaaaaccactctagtacttatttagcactccacttctaattgatttcatctgctggctaagtcaaggcactagggaggtgggagcttcttgttgattaagtagaatcatgtcagaaactccagctaagggccaatggcaaacaagagactacagccacttggtcagtaggcaatcttctatttacctttgccctatgttcaagccatggctttttatcagtggaagcccccaaatttatataaaggagaagaatgcttggggaccttgtcttgatgctaaagtggcttatcaccttacctaaaattgagaatgaggagggactgagggtagagctgatgggctttatggttgctaaagcctcggttatatctcaagatggcaaatttttgattcagcagatctggtaagcaagtcacacaaacttgactttgcactggaagcttgatgatactgaacttcagaaagcagtaaactttctgttaaaaataaagtatgaaggccaaactattttctttttcaccaatgaattgaagatcaggtagtgcaaatgtgagaagagatctcatgagaatgagaatttcagtttccctgagcatgcacttgttgctgctgggtggtaaaatcatagatcagaaaccttggtgctcagcagaaacttcaaggttgcttaggtaaagagaagtgattttttttttccgcttcatgatcaagcatagggggaggcaaatactttttttttctcccctggtgaataatgggaagcaatgttgaatattattgctttcctttttgggaagaaaaaggaagacacattgagggatactttcttcctcatttatgtgtcatatatttccctatggttgaagttcatgacagccttctcatcacaagagttgtgataatgtggtttatagtttatcagtattattagcactaataaataaccctggcaggatgtacagttgattgcaggacgttctctttgcatcttcccactaggtatctagtatcattcctaaacttgcaattaactaaagacattcttaatattacttgaaagtagagattagggtacctgggctctttgcacccttctttgtaaatatcagtgcagagctttcagaattaattgcttcttagatagacgattaatgttcacttagccctgtgcaaagaagtgggtcattctatttattggaatcttgctgttctgtaattacgtgaagagcaaattattatagacagagtttctgtgtgattttgaagcaggtaaatttagcatcattagagaataatctgtagctagaaaaactcaagattctttaaaaacatgcagataatacttaaaacttagtaagttttgttattcttaaatgtgttatataagttggaaaaaacatttcttgtgttagaaatagcatttatggttaaataacaagaaagaaaaagagctggtctccatttatttcttgacattgtagaaatactgtaattgagatttagttacacatgctttgaacaaaggactatgctctaatatttcatttatttagatggagcatgcatttctcttttcattgattaagataacaatcaggtttcccaccttgaaagatgtgatattgaaaatttttctattaaagagactatatttataactcaaaattcaatgggaacctaaggaatctagctaaaagtatatctcacacaatgctatatgggagtgctatgttttataaattcaagtagtctgaaattcttcagttagaattaatttagataaacagagatggcttctaccttgaacttggccattttgcctgccttccttatatgcctcattctgttcattgtaatgcaggctcatttcattttatttcccttcactttattgaactttgcagatattgcatttcttaaaaattgagggttcgtggcaacctgcactcaaccagtctgttggtgcaattttccctatagcatgggatcactctgtgtccctgtgtcacattttggtaattctcttgatatttcaggctttttcattattattatatctatcatggtgatctgtgatcagtggtctttaatgttactactgtaataattttggggtgccaccaacagcatccatatgagatggtgacattaattgatagatgttgtgtgtgttctgactgttctactgactgcccattccctaatcactctcccgttctttaggcctccctattccctgagacaaaacaatattgaaattaatgcaattaaaagccctacagtggcctctaggtgttcaagtgaaagaaagagacacacatctctcactttaaatcaaaggctataaatgttaagcttagtggggaaggcatgttgaaagttaagatagtccaaaagctaggcctcttgcaccagttagccaagttgtgaatgtaaaggaaaagttcttgaagaaaattaataatgctactctagtgcacacacaaatgataagagatagaaacaaccttactgctgatgtggagagaattatagtggcctgtgtagaagatcaaatcagccacaacattcccttaagccaaagtctattctagaggaagaccctaactctcttaaattctatgaaggctgagagaagtgaggaagctgcagaagaaaagttggaagctagcacaggttggttcatgaggcttaaggaaagaagctgcctccataatataaaagtacagagggaagtagaagctgcagcaagttatgcagaagatctagctaagatcattgatgaaggtggatacactaaaccacaggttttcattgcagatgaaacaatcttatattggaagatgttatataggacttttatagcaagagagaaatcagtacctggcttcaaagcttcaaaagacaggctgactctcttgttaggggctaatgccactggtgactttaagttgaagtcaatgctcactggctattcctaaaatcctagggcccttaagaattatgctaaatctactctgcctgtgctctataaatggaacaacaaagcctggatgacagcacatctgtttacaacatgttttactgaatatcttaagcccactgttgagacctactctcagaaaaaaagactgctttcaaattattactactgctcattgacaatgtacctagttacccaagaactctgatggagatgtacaaggagattaatgttgtttttatgcctactaatacaatgtccattctgcagttcatggatcaaggaataattttgaccttcatgtcttattatttaagaaatacatttcataaggctataactgccatagataatgattcatgatggatctggccaaagtaagttgtaaaccttctggaaaggattcatctttctagatgccattcagaacattcatgattcatgggaagaggacaagctatcattaacaggagtttggaggaagttgattctaacccttatggatgacgttgaggggttcaagacttcattggaggaggtaactgcatatgtgttagaaatagaaagagaattagaattataaatggagcctgaagatgtgactgaattgctgcagtctcatgataagacttgaatggatgaaaagttgcttcttatggatgagcaaagaaagtggtttcttgagatagaatttactcctggtgtgaacagtgttgaaatgacaacaaaggacttagaatattacatgaccttagctcaccaagcagtggcagggtttgagaggattaactctagttttgaaagaatttccattctgggtaaaatgctctcaaacagcattgcatatatagagaaatccttcataaaatgaagactcattcaatgcagcaaacttcattgttgttttattttaagaaatcgacacagctacccctgccttcagcaaccaccacctaattagtcagcggccatcagcattgaggaagacccttcatccagcaaaaatagtctgtgaaggctcagatgatcagtagcattttttggtaataaagtatttttagttaaagtatggatgtgtttatttagacataatgcaatctcatacttactagactatactatagtgcaaatataacttttacagacactgagaaaccaaaaaatttgtgtgacatatcttattgcagcatttgctttatgatggtggtctgggactgaacctacaatatctccaaggtatgtctgtggttggttttaatgtacaaaatgaatgaaattggtatggcaaagggccagcttgcctatatggtgactgaattagattagtgtcttagtttgagctctccctaaaagtagaccctgtgacaggattgggtataaggagtttatttgggaggtgatcccaggaagtatggaagtatggagagggagtgaggaagtgaggtggggagggtggaaagccaatcaatagaggagatgctaatgaatggattactgctggagctccttgctgctggggattctctgggaagctgtgtagaacacatgtcagaaatgaggaaaactcagggatttacctgatgtaatgaggcagaatagtggcttccccaaaagatgtctacctcttaatccctggaacatggggatatgttaccttacatgccaagagctacttagcagttgtgattgacttaaggacctggagatggggagattatcacaacggtctttctgagagagaattaggagggttagagtcagagaagaaaatatgacagtgggaggcagagcttggagagtggcggtggggaagagagagagagagagttgaagacgttatgctgttgactttgaagatggaggaataggctatgagccaaggattgcatgtggattctagaagctggaaaaggcaaggaaaaagattctctcctagagcctctagaaggaactaaccctgttgacaccttgactttaggccatgagtctgatttttggcctttctgcctccagaaatataagataacaaatgtgtgttgctttattccactatgtttgtggtattgttatagtagctttaggaaactactatactataccagtgtattaatatacttgcagccctccttggttgcagatggctcctagggtgataccttctctgtgtttctggccaatccactgaaggctaacctgaacaaaggttttgtttgatcaatgaaatgtgaaaatgtcctcataaatgcttggaacgagctcttctgggttgtttcatatgaacagtccaggtttctgagcctctctgaccattccctacctgtctcttctcctggccctttctcctctggctggtctttaaatgttggggaaccaggattatgatcttgacctccagtctcactctgtgatggataattttatgtgtcaacttgactggaccatgggtgcccaggcatttgatcaaacactatattggtcgtgtttgtgagggtgtttctggatgagattaacattttgtattagtccatttttatactgccatgaagaaatacctgagacagtgcaacttacaaagaaaaagaggtttaatggactcaattccacatggctgcggaggcctcacaatcatggcagaaggctaaggagccaaaggcacatcctacatggcagcaggcaagagagcatgtgcaggggaatggccctttataaaaccaacagatcttgtgagacttaatcactatcatgagaacagcatgggaaaaacccacccccatgatttaattatctcccaccatgtccctcccaagccatgtgaggatagtggcagttacaattcaagatgaaacttgggtgaggacacagccagaccgtatcgcatttgaatctgtagactgggtaaatactctcctaatgtaggtgggcttcatccaatccactgaaggcctaaatggaacaataagtccaactgccctatgaaaaggggcaattcctcctgcctgacagcctgaaaactgagacattgtttttttccctctgccttcgggcatgaaacaccagcttttcctgggttttgagcctgcaggcttttggactacatcagctctcctggttctcaggcctttggactcaggctggaactaaaccagtggctctcctagatctccagtttacaggcagtagatcttggaaagtctcagttttcattatcacatgagccaacgccttataataaatcattctctctctcttcagcattgctcattctgagttttaaggttcaccctaagatttctgccactcagttggttgttgcccagtggagagctaaggatgctgctaccctggcacacaggactgagttggaaaagtccttggcgaacatggaactaagagatccagtttattttctctgaatgagaaagacatatacaaagagagtcccctcaccccacacacaccacagacaggaagagagagacagggagagaaagataatagagcaacagaaccaggcagacatccagacacagagtcatagagaaagagacaaggggagggagagaaaggacagacagacacaaatcacaatgtgatatgcagagacacaatgagagacagagacagagggagacacatacagaggcagaaagagaagcacatttgtccgagggaacagagaggagaaggtaaaggagtcagaataccaggtgagctaatacacgctgcctcccagcttgagtatcccccatatccagaactcaggaaagccccgtgagctgtgataaggtctccttggcccacctgccctcacccctccccagagtgacccatgggtgtactgaacaggctttagagccataaaggtctgagttccaattccagatcagctactctttagctgtgtgactgtgagcactacttgagcttcattttcgtcatctgtaaaatagtggataatagtatctaccttgcagaatttttgtgaggtgtttaacaaatcttaattattatttcagacaaatcctggatctcttctggctcccatccctacccctttagtaaaagggaatttatgcatatatctgggataaatatagtccataaaactttgtcactggaaataaatgaaaattaaacatactcctctaaataaacagtttgggatacttttcagccacagcccattaaaacacatccattggtaggatgcatggaaatgttattttatttttttaaattatattttattattatttttttgagacagagtctcactctgtcacccaggctggagtgcagtggcgtgatctcggctcactgcaacctccacctcctgggttcaagcgattctcctgccttggtctcctgagtagctgggactacaggtgcatgccaccatactcggctaatttttgtatttttagtagagacggggtttcaccttgttggccagggtggaggaaatgttattttatctgcagctgcatcttcgatcttcacacgttttttgttcagcagaaaaactaggagagtggcaagttgctacacctgggactcgtatacacaagagggtgctggcgagcatgagagaggatctatttttaataacccatgactacttttttataaaaaaattagtttttaattgacaaataaaaatcatatatatttatcaacatgactattgaaattcaaaacaacatgatgttttgaaatatgtatacattgtagaaaggctaaatcaagctaataacatacttagtcttacctcacatacttagcatttttgtggtaagaacacttaaaatctactcttttagcaattttcaagaatacaatacattgcgattaacaatagttatgtgttgtacaatagatctcttgaacttattcctcccatctatatcctttgactaatatctctccaacgtgaccccctcctacacctggtaaccaccattctctgcttctgtgatttatactttttcagattctacatataagtgagattgtgcgatatttgtctttctgtgcctagcttattttgcttaacataatgtcctccaggctcatccatgttgctgcaaatgataagattttattccttttttatggctgaataatattccactgtgtttatatacagtaaccacattttctttattcatgtgttgactggcacttgattctgtgtcttgagtattgtgaatagtgctgcagtgaacatgagagtgcagatatctcttcaacatactgatttgttttcctttgaatatgtacccagtagtgggattgcagggtcacatgatagttctattttaattttttgaggaaactccatactgttttctataatggctgtaccaactgacattgccaccaacagtgtacagggatttccttttctttacatcctggccaaaccttgttatgttttgtctttttgataatagcctttctaataggtgtgaattcatatctcgttgtacatttaatttgcttttcactgataagcaaattaattagtgatgttgaacatttttcatgtactttttgaccattagtttgtcttttttttttttgagaaatgtctattcagtccttcccccatatttgttttcttgctattcagttgtatgagttcattatatattttggatattaaccccttatcagatatatagtttgcaaatattttctcccattctacagcttgtcttttcactctgttggttgtctccttggctatatggaagctttttagtttgataaaatcccggttttctatttttgtttttgtttcctgtgcttttggagtgatagccaaaaaatcattgccatgtccagtgtcataaagattttcccctttattatcttctagtagatttataatttccggtcttatatttaagttttaattcattttgagatgatttttgtatatggtgtgagatgaggctgtaagtttattcttttgcgtgtggatatccagttttcacaaaacaatttattaaagagactgtcctttccccattgtgtgttcttagcacctttgttgaaaatcaattgatggtaaatgcatgaatttatttctgggctcttgagtctgttccattggtctatgtatctgtttttatgccaatgccatgctgttttggttatgtaatatattttgaagttaggtactttgatgcctccagctttgtttttttttgttgttgttgctgttgaagattgctttagctattttgtggttccatacaaattttaagatcgtttttctctatttctgtgaaaaatgtcattgggattttggtagagattgcattgaatatgtagatggtttttgattatatggacatttaaataatattaatctagtctgtgaacatggggtatctttccatttatttgtgtcttcttcaatctctttcatcaatgttttatagattttagtgaagaagtctttcacctccttgattaaatttatgcctgagtattttttttgtagctattgtgaatggaattgttttcttgatttctgttttagataatttgttggtaggatatagaaatgcttgatttgtgcatgttgattttgtatcctgcaaatttactgaatttgtttatgacttctaaccatttttttggtggagtttttagttttacatttttatacaaataagattatgtcatctgcaaacagcgacaatttaagttcttcctcttattattttgagacagggtgttggtctgttgccaagggtggagtgcagtggcatgaacacagctcaccgcagcctcaacctcctgggctcaagcaatcctctcacctcagcctcccgagtagctgagactacaggtgtgccactacacccagctaatttttgtattttttgtagagacaaggttttgctatgttgcccaggctggtcttgaactcctgggctcaagtgagccacccgcctcagcctcctgaagtgctgggattacaggtgtgagccactgtgcccagccaaagttcttcctttataatttggatggcttttatttgttctcctacctaattgttctggccaggacttccactgcaaagttgaatataagtggcaagagtgagcatccttgtcttgttcctgatcttagaggaaaggccctcagctttttaccattgagtataatattagctgtgggcttgtcatatatgacctttattcatgcctactttcttttctgtgtacctttctctctactagaaggagaccagaggccttattatagcactgtgctcttggatttgcattaaataaaatttcaagaaagataacaaaacagtgggattagagtgaatttgtagctgtgggctgagctgtgagccaactggaaaggctctaacttcttgtgttcactatcagcaatctactgtcaactttagtttgcaaaggtttatggaattttgggggttttgttgtaaaatgtgtttttttccttaatttaaaagtagtgagaaattgtgatttgctaaatctgaaaaatacagagaataaaagacaaatcatcgataattctactacccaattaatcaatcaactgaattgattaattcagttaacattttgatgtgcattgttcaaattttacacgtaatattttttatctacaaactcaggcttacattatactacactgtttttttcctttgcaatgtgtaagttgaacaacatcttgccattaagtattttaaaaataattttattttcatataatactctatagacataacctaaggttttgttttcccttgagtgtttaaaccaatcctgttttgttggatattaatattgtttacatgctcctttttattataaatattttgtgaacatgatttaaacaaaatttttgtgcacattcatggttaatcctttaagatacatgtcttctataactggagcttcatataaaacattttaaccttaataaagcttttgatatggattgcaaatctcccctccacctctttacctttgaaaagctgtatccattcatacttctttaattactgtgatgtttgtctattttttaaaaattacagttgtcagttttcctatttataaataatcatctttaaattttattaaatttaaatttaaattttattttattttttgtagaggcaggagtctcgctatgttacctagactggtctcaagcaatctgcctgccttggcctcccgaagtgctgggattacaagcatgtgccagtgtgtctggccatcatctttttttttttttttttaactaaaaatatttctttataattttctttcttcttgcattgcattctcttatcattttctactccttaagttttaaaatgttcattgcataaaatttatatttattgcaacttgaatgttttaccgaaaactgtttacaggatcctcatgaatgagaatcatgcttactcaaagattgtgaactcacttaaataaatattttctgaaagaaggaaaaatacttattaagcacctgagtgttcaatactatgcagaggaccctggcggatacctgagaagcacaaggcatggcgctccggggtctcatgttcttagatggggaaatgtcatcaagctcacagtctaagaatagtgtttcctcaacctttttagacctgtgcccaccagaatacttactcactatcctctctctccagccaagaatattttgcctggctttcccactctgtgttatgaattactgaactagatcaatggaacatgctccttcagatatctgtgcacatgtaaacatcttgcactgatgtgccttcttgccttcagtggagaatttcttcattagataatcatccaaagtaggactctgtgaaatttactctaagaacctaagatgtggacagtttaccggtgaagcctggattgataaggcctaagttaaaatgcttctcttggcgatgagcttcctacagtacacaagcctatcagtgccttggaccctgggctggtctttgcctccttttcctcttctccaggctatccctattctgcaccatcttgcctcctccactaggtgctcatattaacattcctgcctccacctctcttttgatttgccctatttattgtataatacccaactgagtccctgattgcaagcatcttgcttcaaaggttggtgggttctgccttgcactctagcaactggctttcctcctctgttcttcactctttagtccctacattgctgcttgtcatccacttcttatagacttggtggcctaacccctttctcactggaattaggtcttataacttctgctgacccactttagagcataatgcctgatcgaacacaatgtacataataaatatttgttgaatgaacaaattggaaacattatcttgcttctatttaaatatgtgtcatgggcttaaatgagacgtatttacaaaatactcgtattttacttacctttcttcccctcctgccctggatttctgtactaaacactttgcatttgttatttctaatcttcacaacaatcttgcaagacagatgttgccatctctctatataaggaagctgtgggtccagagtttctccagattcactcagtgaatcagcggtggatcttacttctccacggaaccactcctagaatcactaacccctggccttattcttgaggttcctaggctttcttcttctgacctccccatgaccctgctcttatcctgattgatcaccaccgtctttgtctgaatccctttgagacagagggagctaggagggaaggacaagagagaggtcttgacaagggctctgcatgagagattaaagccaggacctaagggaggtagaagggccaaaggtagcttcacattttcaacttgaaggagcagaacagtggtagtatcaccaaaagaaattggtagttcgaaagtcttactcctgggttttaaaatcaggtttgagttccaatgactttctttctttctttcttttttttgagatggagtcttactctgttgcccaggctggagtgcagtggtgtgcccttggctcactgcaacctctgccttctgggttcaagtgattctcctacctcagtctcctgagctgcgattacaggtgtctgccaccacgccttggtaattttttttgtatttttagtagagacggggtttcaccatgttggccaggctggtcttgaacttcacctgcctctgcctctccaagtgctggggttacaggtgtgagccaccgtgctcggccccaatgacaccttactctctggttaaccatgagcaagtctcttagcttcccttgagccccagtttcctcatcttttccctgagcataatctgacctgactttcatgtttgtcattggtttgctgttcaagtgatgcaatgcatgtggcatgctttgtaaagggtggagctgtgtgcatgctagccatcatcagtattttcagatatattattactagagtcagattgtaaatggaggcaccgacttcttggttaaagtatatctatgccaggggccagcaaactgtagtccatggcccatatgtagcctgctgcatgcttttatatggctcctgagcaaagaatgcgaatgctttttacattttaatagggttgaaaaaaatcaaaagaaaaatatttatgacatttgaagattacatgaaattcaaattttaatatccataaataaagtttattggaacatagctgaacccatttatttatgtattgtctacggccacttttgtgttatacaacatcagagttgagtagttgtgacagagactatacgctctgcaaaacctgagatattaactatctgactcttcataataaaagttttccactctgtttccttccttcctgtctgttgaagcttctgaaaagcagagaaaatttcaggaaatcttaaaagttttttttacacttatttcttttgtttgataatgaaggtaaaatgagaatatatttgtctttttttcctcctgtctgatgcttttaaaagtaaatcttcattcaatgttgaaacattattatgactttgaaaggtaattagggtaaaaaaccaccaacaaactcaaaccaaccttaacgtggccattctaacacatatttcaaaaatgaaattgatgcttccttctactcttggcttatttatgtattcccaaatggctgtaactatggtctacataaaatttcatagttgatcagcctttttcagttggctttgtgaatattacttggtcattgcttttatttttcatagtggttgtattgctatagaatggtacagttcacaagttatatgtctttctgaaatacttcttattcttgcctttcatccaatccttgcttcttcaaatatgattgcaaatgcttctctttggagccagctctgggcaacctgggcttgcttattttcccttcctcccttaatctctgccctctgatattgattcaagcgcagcattcccactgagcttacaaactgagaaagatgagaaataagccaaagaaaaagtgtcagagaataaatgaacttgatattttaaaggtgattttcgctttgcccacacctccccactaatcattcaataataatagaaaaaacatttagcaaacaaaaatattctccatttatgtaataatgatatgagggatgtttgtgctctccattgcatttctaaataagtagaaataaaaataaggccattatcgtcattatgttatattgttaatccttttactgtatttaatacaaaaaactctttaatttgtcaaattaacttaatgccatgaattataagtaaaagatggttatttcattttattatcttatttttttcctagtgagaagctggatggaaaaattaaaagacaaggtaagaaaagtattttcttccattggtttatttttaggggcatatagctttattaagcaaaagatcttcagagtagtagggctttggtttattattctgattctggtaatctatctaaagattcagggcaacttgcagtacctgcaaaaaagaagcccttccctttaaatcccttcttttagaatacaggagacaccttattctttggagcaccattcactcttataaaagtgtaagaatttaccttgtttgatatgctccacagaataatatcattgacatatcaaggtgaagtttctttttgggaatttcaggattgtcatctcattccatctgtttttttttttttttttctaatatgagactctctaggtgtgtagtttatcacagtgctgccacttagtaggtattgattagctgacctgtctggaatggccaactacagataaagattttctttttttgcattaattctgccatctacatagtgattgacaacgttaacttccaggttgatattgttcattttcttttgattttcaatcctctccaagttttcacttttggttacttggtaattttaaactgaattactcataaagatattgatggagtcctgaattccttgttgtagccagtgtcaggtcccattattacagcccagctcagatttcactgggatttcagctaaatggaaatcttttcagtggtcagatattgagttgccttcaattcaccagtctcaatatatttctgttgatggcattccatatcttttttattacctgcagtcaatttccctcatatttgaattattaatattattttgctacagcttaatattaaagaatacaacattcaattgcaaataaatttatataattgtattaattctccttactatttctgcttctcagtaaaatttagcacagatagtttttgagaatgagactattttgtgttcatataaattaaaagtgacattgaaaaattttaagcctgttgaggtcaatggaggccattttggtatttgtaatgggtccaattgtgaagagaggtgggctgggatcagatggactgagaaaaacttggggggaagaaaagctcaaaagtttagaactggatatatttacattggtagtgtttcttgttattttttcagggaaaaatatgatgcaatgaaaattaaacaggatttgttatgttaacctaggaattcatatatcattgtttccatagaaagtatcctgagttatttaagaaagggaccaaaaaaaaaaaggcttaagaaaataaattttgtcatgtaaacagttttgaaatttgatattgttaaatttcttatctgcatgtatatatagctttaccattccctaaaaaaatctgtacagctttagagaaaggctatatttttttattcctatgaaccctttattcttatcttgtctcaaaatcttggtacctttttcttgctcctttttatcaagagaaataattttgtatgtgaccgctagttttctggcttattttgaagtttttccagctttctcatcactttcttccgtagtatttttttcagcatattttaattaaaaaaattgtaagaagaaaattttgaattccaatagtgactgtcatatgtgcctatatcttcatcttttgactcttggaggaactctttaaatcttccccatattccctgccttcttccatgcttgaacgattacatgctaatttgatatgtatattttatatgtatatacacacatatgtacatatatcttcgtttaacaagtcttacaggtgatcctgatacatgctggagtttgagaagatacatgtacatagatcttacacacatatatatatatacatatatcttcctcactctctgtttgtccatccatccatccatccatcatctattaaagtcttgttagtcattatcttcagaaactatttgagtacaaaactctttgtctctttgatgaaagtcacagaatcccttagaactaaaaactaaactacatgcccaacctggctcttcaggcaagccaggacactctgtcaggttagcttcatttaattgttccagtattgagtctctgttggttagctggattctagaaagggtggtgttttcaaaagttctgaattattcctgttgttgttgtagtttagtctttctatattaagattaagatcagctgtgaatgatagccaatccgcccccacccccaatgacaatggcttagccaagatagaagtttgttatctctctctctctctctttttttttttttttttttttgcagagtcttgctctgtcacccaggctggagtgcagtggcatgatcttggctcactgcaacctccgcctcctgggttcaagcgattcttatgcctcagcctcctgagtagctgggattacagacatgcaccactatgccaaattaatttttgtatttttagtagagatgaggttttaccatgttggccagggtggtcttgaactcttggccttatctgacctgcttgcctcggcctcccaaagtgctgggattacaggcatgagctactgtgcccggcccctattgtctcttgtttttaagtctggagatagtcacaggttagtggggagttacacagtgagagcggcccaagcttcttctatgtgatgctccaccatttgtgaccttcattcccaaggtcacctcatgatcctggatggcacttctgacttagctatgtgcgtattctagccagaggaaggaggaaaaagggggaggctaatgacacccctttctttttaaaagggaggataaaagcactgtcattcttaaaggggaagaataaaggctctgctccccttttatagaacgtatcttgaaattttcttatgccactgacatatcatcttcttggtgaaacttaccacatggttgtacccagcttcccagcaggcaagaagtcatttttattttgtgcccagtcccaaactggaagctcttttgctatagaagaagaaaagaatggatattgaaggacacttcctgtattaattccatcattaactgtcttcttggctaatccaagttggtgatccttgttaggcaagttttaacttggctacccattagtatatcttctcagcatctaaactgattttcaaaactgaagatgcataacaattttaattgtgtaataaagaccagacctgttgttatatcaataaaaccaatctatttcttctagctttaggaaaccatccagaaaatgcatggcccagactatacttcatagcagcgattctcaatgtatggtctggactagcagcatcagcatcacctggagcttgttggatatgcaaattctcagactccaccatagacatacctcccaggaactctgggaatggggcccaacaatctgcattttagtaagtcttgcaggtgttcctgatccatgctggagtttgagaacaaccattgcacagaaatcgtaaggatgctgtgtcaattctgcagactttaggcagtacagcatatcatttggtttcctatggctttcttgatgacagctattctgaataaatatgttatcctaagaaggaaggatacctttcttgttgacttcaaacaaacaaaacttcaaaaccaaacaaaaacaagtatatttagcaatatttgacttataatttactttaaaatttaaaactttatttggaaataatttaaaatttacagatgagatacaagagtaagaatggtgcaaagaacattcctacacctttacctggatgcgcctgctgtttgcattttaccccatgtgctttatcatttgctgtcacattctcctcttttttcctgaaccatttgaggataagttgcatgtgttgtggcctgtttacccttatatatttcagtgtatatttcctaagaatgaatatatactttaagcagatctctgtgcagttattagctggagtatattcaatatcgatataatacttttatggaatctggcattcatatttcagttttttcaacaaacccaaaatatccttcataagagcattttttctcctctctcacagaatccactttaggattatgtaacacatttagttgtcatgtttctttaatttcctttaatctggaacatttttatagccatcttttatcttttatgacattaacatttttaaagaacacagtctttttaaaaaacagaatatactttaagtttgttggatgttttctcatgattagattcaggttatgcattcctggttggaataatacattggtgcaattgtgtctttctcaaggtgtcacatctcggggcatgtgtatatgcaataatttggacagagaaaaagtactaggcagaatttataacttaaaactgtgtgcatgttgtcttatttattttcattgctgaagaaatcaagtcatgagcaattctaacagaggagcttattttcccttagtcagttttgaaagaagaatagttaaattatatcttaagatggtacgttgctttgctcccatcttgtaatttacttgtaatttcaaagcaaggcagatgccaccaaaaaagaaaaacaaaacagaggagacaaaaaagagagatgtaactgagtttttgtgaaatctacataatttagagctctagctaaatgtaaccattccacaatgaaagtatactttaaaacatcatgttgtacatgatgagtacgtacagttttatatgtcaatttacaaatataaatataaatttgaaaaaataaagctccataagtaaaaggcttaagtgtgtcaataagaaatgcagaaatggtgacatatttgagagtaccgatgttgttggtatggggttaagttacagagtatgaggagacatgcacagaggctgatcaagttgaccttccaaaagttcaaatgttactgtacatgagaagcctaatcatctgggcttaaagtttatcatatttcaacatgtgtgtgtttccatacactgggaatggtcatgaaaaataattcacagttcttgtgtgagcaccaacactttgatgttgttcagtattttcccacatggaaaattgcagagtttttataagccttaagtagatttggaagaactttagaaaaagtttgggcttctacaaagcatattaattaattaatttatttatttatttatttttattatacgttaagttctagggtacaggtgcacaacgtgcaggtttgttacatatgtatacatgtgccatgttggtgtgctgcacccgttaacttgtcatttacattaggtgtatctcctaatgctgtccctcccccctcccctcaccccacgacagggcccggtgtgtgatgttccccttcctgtgtccaagtgttctcattgttcagttcccacctatgagtgagaagaaggcatattaatttattgaaaaatgtctaggctttgtttcttggggaaaaatacttagtatgctgaggaacagtgctattgttctcaaattaatggaagaatttccatattttgccctaagtgaagactctgggaggcagaaacagtgagggagctggctgagcacaagaggaaagtgctgctgtttcataaactctgatgtattctttggatgtcctgctagaaatgttgggaaaacatcacagcctgcatacttcagcactttcaggaaaacttttatcctctgtcctgctccatttttggctcactttgaaaaatactaaaacagtttgtgctgtattaaaaatttaaaaaatagtcatataaatgaatagacttatgaacaatttaaattgtttatgtattttgcatgggtagacttctaagaagggcttcttgggaggcaaaccattacctgtgttcccccatgaaagggctataggttagtacataccctgttagcccataggagtcttattggccaaggtgtagggatcactctgagtacaatggcctttccctgagcttaggtaggtcctgaggtacttactcaagtccagggatcatcctgctctctgaggataaaggtatccatagtgacaccttggagaccctggagacatcgatgaatttgacacttccaggatcttttggaggtatcgtgttgttggcagtgatctttgtgtacttctctgccacttcctttctttggggctgggacattcagaattccctcccaacctccaactttgtcttgtctgttgggaaggtgattccattcacagaaatcagtccatccattttgtgtgcacactctccccttgtccttgtcttttctgcctttgctccaacatctaacattcctcctcttgcctttgcaggccttttttttccttgttttcagaagccctccccttttacccacaagcattttcatctttcttttcttactgtaatgcagccaagacagacagagagggagaagagaggaccaccactgtcaaggccctccttttagcccccagcaaacacactattccagaggacactttgacctcctttaagcttgactctggctgatatttccaatttatagcattttagtagtcctggcattctcagaggcttacttcatccatctttaatacaaattatatttctgtcatcagaacctgaggttgacaaatgtgcataagctagtggctggaggtgccactaggtctggctgatttatttcaatacttctatgtatttgaaatatccccaaaataggcctgtctctctctctgttgccttctccctaaaaagttactgtattttaggcttagttttatcttacaacaaagcagacaggagaattcaaagtgtctaaaaacctgctgccatccagcagaaatgagaaacccagggcggcgcgctctgtttagcatcctaatgtgtgtttccttttctatgcaggctggttacatgtctgggatgctggtgcctgtaggggttgggatagctggagccttgttcatcttgggagccctctacagcattaaggttatgaatcgccgaaggagaaatggcttcaaaaggcataaaagaaaggtatggaaccaacaacagcaaaacctaatcagtttcagaacattctttgcttatctaatttacttaataaagcacacggggtttaatgatgcttgggtggtattgacgtgcccagtggtctggattagctgctgtagctcctgccttggcttcgtttgttagcatgtcttttaaagtgggcctgtttctcagctttgctctgtggcagaaagagtttaatgcttttctgttttctttctctagcagagagaattcaacagcatgcaagatcgagtaatgctcttagccgacagctctgaagatgaattttgaattggactgggttttaattgggatattcaacgatgctactattctaatttttattttggagcagaaaaaaaaaaagaacaacctgccacattgctgctatcaggccgttagtcctagtgtctgctgggtgctgggtagtagatttttcttgtactgagcagaaatggcatgttgtatactaaacgtatcatgcagtatttggttttattctgtagtgaattttccacaaccgtgggctacaactcataaatatgcaacatatatgtttttcagtaggagttgctacattaggcagagtaaatattttgtagttttccacagtgtcttttccttggtttgaattacctgcattgagaataatgattgttgccaccaaggcatgcttgactctgagatataaatcttaacaaagaataacttctcaagatatactctacctacttgaaaccacagggttgtgggccatggtacatactgcatttgcatcaaactagcagtaactcagaatgaaatcattttcattaagaagctctctcagcatattaggattatatgtagatttgtatgtattttgcattatgtacttcagtctcctagttttattattctcaccttccgttttattcttggcgaggaaaaaaatgcactagaaataatacattaaactgactcttagtcttaatgtacgtttgctgtcttaaatagtgtgattgagtccaacagactcaatcatacatgtcatacatgtttatgattaagagatattctttttgtgtgctagttgattttgccgagaaaaaatgaagaagaattcaagaatgagatgagggtaggtaagctctcagagcatttctgtctgccatttggttctatgcttatgtggctgctaatgtgactaattcagagtgttgtatttccacatctgtggactccaccatggaaaaggtgggctaccattggtccttatatggctttattagaaaaatagacattctatcgtttgtctgcccagtggccagagtcctggtgaacaacagagctcatgggaaaccagcctctctcagggcaccccgctatgaggatattgaaatatgttcaatcatttctcatctcccttggaatgtaattccctgccctatacaaaataggatattccaatgcgctatttgaatctagggattgaggatttgtagttgagttttggggtaaaggcttggctcattgccatggaagaataaaagttatttattaatagtgctgtcactgtggagtacttgttggtgtttattagaagttgtctactatgttcaagaaaagttgcacagttattcctcagagctactatgtgaggtaggacatatgtttttattctcccatttgactgaagatggaactgaagcagagaagtgaaatgattttccaaagttgtacccagagagaatcagcgtgagaacccacttggacccagccatgcacccttatcttcagttcagtaaccttttcatagactttggtggaagcagtgacatttcagggtccttctttaaaacaaaggttctaagggaatagcagggaatagctgcctggtcaaggaacgtttgatgtgaatttccatctttgttaaatgaatgatgcctttctaatttctctcttactcacacacaagttctttccttccccatctcagttatcagttatttcctttacagtcatacatttgagtatcacctctatggaactaataaaccttcctagaatccgtttttgaataatgtctggctcacaagaacattttcatcaaataattttaactctttttgccaagagatgtaagcaaatatgtgaaactgatcaagagaagtgaaaagctctgtacagtcaaatagaagttagcaactggcaaattagctcaaggtcagattttaaaaatagaattcagaatcatgacactcagaagtgatttgtaatttcacgagtagagacatcctgatattgcataagggatgttttccttttctaagagcagccctttgctgtaagtgtttctaaggttgggagtaagtgagcatggtgtggcattactgtctttgtattcttgacaaactatttgttgagaatacgtcgtaggccacttaactgagtagcacatgggtgatactagtggccattattgccttgggctactcaatctatagtcttaactaaaccatggctaataggttgaatcttcaaactgtagtgtaataaattcccaaataacagacccatattttcatttctcagaggtccgtgaggatgacatgtatgtgaattttggcttaacacagtatctctgtggtcctttttgggacacatgatttcttaaaggaaaagattatacttagagtacatttctgtacaaacccatttaaacatctggattgagtggaaggtcggtggatggtacaaatgacatgtttattttggccattttccttgctgctggtggctgtggaataatgattttcagtggttggcttagattattaagaggatcagaaaaggttgtcatattttaaacaaaacctgggattagagatgctgctacgagatggaagtgctggccagtggttatatagtggtccagttgagaagtagtaaatcaggcacttgtaggtttacccaaaataagtgacaagtatgcttatatttgcacaattaaaatagaaagtcaactttttattcaaacaactaggcttaaaacatttaacagtgaaacaaataaagctgtctggaaaggagggctgattcctgcagcttcagttgtgaacaccatccccactggactgagtttacccagccagaactgccagcatcccaccgcaggggcaggaactaggtcttcttctcggagggagaggcagtgtttccagaatttgcaggctcataaaaatcacctgggacacttgtgaaacatacagatctcaaggccttgccccagacctgatgagttagaatctccagggaaggagcttggggtattcatgccccagcaagcttgggaacacagctaggagggatctatctagagaaccacttccttatgatagagacaaggtcatttaaagtcacaatgagataccacctcacacctattgggatggctattattcaataaaacaaaatataacaattgtttctgaggatgtggagaaattggaatccttgtatactgtgggtgggaatgtaaaatggatagccactgtggaaaacagtatgaagattcctcaaaaaattaagatagaactactatataatccaactatcccacttctgggcatatatccaaaagaatagaaatcaggatcttgaagaagtatctgtacgcctgtgttcattgcttatgcttataagcatattattcacaataagcttataagtattattcacaatagccaagatatggaaattgcattgatagatatgcaaatatctatcaatagatgaatggataaagaaaatgtggtatatacatacagtagaatattattcagccataggaaataaggaaattctgtcattgtgacaatatggatatacatggagaacattatgctaagtgaaataaactagtgacagaaggacaaatgcatgattgcacttctttgaggtatccaaaataatcaaactcataagggcagaggcaaaatggtgcttgccacgggctgggcaaagttaaaggtggggagatgttgattaattatgtttcagttatgcaagatgaataagttctagagctctgctgtataacatagtgcctatagttaacaatatcgtactgtgcagttaaaaatttgttaagagggccgggtgtggtggctcacgcctgtaatcccagcactttgggaggccaaggtgggcagatcatgaggtcaggagatagaaaccatcctggctaacatggtgaaaccccatctctactaaaaatacaaaaaa

SEQ ID NO: 2 - DNA, 24 bp, synthetic construct
  source 1..24: mol_type = other DNA
aattcaagaagatgggagcctctg

SEQ ID NO: 3 - DNA, 24 bp, synthetic construct
  source 1..24: mol_type = other DNA
aattctcatccagcgagtctgcat